SEQUENCE LISTING (106 sequences)

SEQ ID NO: 1 - DNA, 11080 bp, Homo sapiens
  misc_feature: Accession Number NC_000017.11>NC_000017.11c58280935-58269856 Homo sapiens chromosome 17, GRCh38.p12 Primary Assembly
gacaatatcaggtgagctgtggaggtggggtccttggaagctggatgacagcagctggcaaggggataagagagcagtgagcccctccctcaaggaggtctggctttatccatagacagggccctctgaggtggggctgaggtacaaagggggattgagcagcccaggagaagagagatgggggttcccttcttctcttctctcagatgcatggtggacttaggaccttgctgggctgggggtctcactgcagagatgaagctgcttctggccctagcagggctcctggccattctggccacgccccagccctctgaaggtgctgctccaggtaacagttcccaaggtgggagaagatggtgtgttggggtggttgtgttccagagaccccttttctcagagcaggccttcctagctctggggcctgatagggggttggggcccattcctgactttgtgatccctgctctgggcagctgtcctgggggaggtggacacctcgttggtgctgagctccatggaggaggccaagcagctggtggacaaggcctacaaggagcggcgggaaaggtggggcacgaggcactgcccagctctgggcaaggatgtcccaggcctttcagagaagcagcaggcagcagggagcttgaaggtgggagagggggactatggggacccatgtggactccctttctgtctgtctgtccctgtgtttctggatgggagaacagcttaaaagccatccttcccaaagccttgcctctgtctggacacccaggtccatatcctttttataaaaggaagccttcctcccacccgtgggcctgcctgcatggtcctgcatgtctgcccctgggctggggctctgctgggtgggtctgtatcccttctctggccctcactcctcctttctccaagcagcatcaagcagcggcttcgcagcggctcagccagccccatggaactcctatcctacttcaagcagccggtggcagccaccaggacggcggtgagggccgctgactacctgcacgtggctctagacctgctggagaggaagctgcggtccctgtggcgaaggccattcaatgtcactggtactgttgcccatcacaccccaggtccctgctccactagtgactcctcctagggaccccagccgtctctcagtgatcccctcaacttcttcctccagggagtctcaggagtctccagggctcctcagcctcaggttgcctgggataggaagtgaggcggctcagctcccccattgttctttcccccggcagatgtgctgacgcccgcccagctgaatgtgttgtccaagtcaagcggctgcgcctaccaggacgtgggggtgacttgcccggagcaggacaaataccgcaccatcaccgggatgtgcaacaacaggtgcggctggctgggggtggctgcaggaaccgggctcagagaggcgtcccggacgccacaagcctcccggtgtcagcgccctgtcctcccctgcagacgcagccccacgctgggggcctccaaccgtgcctttgtgcgctggctgccggcggagtatgaggacggcttctctcttccctacggctggacgcccggggtcaagcgcaacggcttcccggtggctctggtgagcgccggcgggcagagggggcgaggcccggccacgcggtgcgcggacccaggcgccagctgatctccgtgtcccgcaggctcgcgcggtctccaacgagatcgtgcgcttccccactgatcagctgactccggaccaggagcgctcactcatgttcatgcaatggggccagctgttggaccacgacctcgacttcacccctgagccggccgcccgggcctccttcgtcactggcgtcaactgcgagaccagctgcgttcagcagccgccctgcttcccgctcaaggtggccctgcttcccgcccactgcctgggttgggagaggggagattgtttctggaaggggccatctcccttctgtgcccaggtttcctttcccagacgctgaggaaggctggccctgcctcccttgtgtccacagcactggctgctcagctatgacctgtctccttcctgcgcctgggctcagccagctggcagccagggccgtccatttgctcactgcctctggcaggccaagggagagagggttgttcctccttggctgacaggagtcctgttggtggaatcactggctctttgttaggttgggagagggttctgggaggagaccttctcagtgccccccaagcctccatgctcagtcctgtttccgccccactgagagggcagccccaacccccatcacacagaaagagagagcctgggggtctggtgggtgccgtgggacagggctgggagtgggcagggtggtcgaggcactgctgagtagggaaggaggaggagagaaaggagagagcgcctgagagagggagagacagacaggagaatgtcagggccagagggagagcaggcacagcagagagaaggggagagagacgggcgacgctttagtgaggagggatccgaggcctggcggtgcccagacccaggggttgcccaggttcccagttcagtgttctgctcattaaccctgcacctcagaggctgttgctgatggcgccctaggcagcgatccctttcgggcctccggaggcctctctgcaggttgagggtaccagaggtcctgagggcagggggagatccagttctgcctgggcaccttccttgccctcggtgagccagtctagcctctctctgtgcctcagatcccgcccaatgacccccgcatcaagaaccaagccgactgcatcccgttcttccgctcctgcccggcttgccccgggagcaacatcaccatccgcaaccagatcaacgcgctcacttccttcgtggacgccagcatggtgtacggcagcgaggagcccctggccaggaacctgcgcaacatgtccaaccagctggggctgctggccgtcaaccagcgcttccaagacaacggccgggccctgctgccctttgacaacctgcacgatgacccctgtctcctcaccaaccgctcagcgcgcatcccctgcttcctggcaggtcagctttggggtggggaccagaggtggcataggagatgttccctgttggagccacagtgagtctgtttgtgagcagcttgtgggtttgtacttagagagactgtcctcaccagccattactatcaacttcatgatattaatccagttgccttggtaacaagtcagatggagccagaaaagcagaaaagcaaacaacctctccaaccctaagatggagacccaggcatggctgaggagcctctgtccatcagtccattgctttctctgtcccttttttcccttctggactctggaagacaagagagtcaaatccctcaggggcagggaactaaaggacagagaacgctgtggtgccctcaccctctagagcgacactatccaatgtggtagccactagctgcatgtggctattttaattttagttattttaaattaaataaacccagtttctcagcggtgctaaccacatttcaaatgctcaattgccttacagtctagtggctccccctattgggtggcacagatgtaaaatattttcatcatctcggaaggttgagttggaccacactgctccaaaatatcccctcctgcgcatcttaattacccataataagagatgatgggattttaaatgtaaacaattggcagttttggagacacaattggggcagtgatggcagccatgaccagacattttccagtgttttttttttttctaagacggaatctcactctgtcacccaggcttgagtacagtggcacagtctcggctcactgcaacctctgccaactgggtttaagtgattcttctgcctcagcctcctgagtagctgggactacaggcgcctgccaccacacttggctaattttttgtatttttagtagagatggtgttttaccatgttggccaggctggtctcaaactcctgacctcatgatctgcccaccttaacctcccaaagtgctgggattacaggtgtgagccaccgcacgcagccattttccagtgtttttaaatgtaaacccaatagtggacctcaggccctttcctctggcctccctctgcctcctcctgatggttcctgctggtctctccctggctgggaccctgggaggaggatccaggtcggactttctgcaagagatgcccgacctcttctgggctagttaggcaagggcagccctccttagccagactcagctgcccacagcctggctttgatgagccccatgtcccaagcctgggtcattatgacctctagctgttcatggatggagatgctgctctcagagaatttcctttccaccagcttctagagctggcaaggactgaccctagtgtcctgaccctagccctctggcccctgcagagaagggttctgggaggactaggggaaccaggggggatctgcttagttttcttgaacagtctcaggtgtggactccacatatctcatgtcctagccctctcccctggggcaggctgcaccattggcatgtagtaaagggcagggctgacaactctgttcaaatggtagtggctttctggagtcctgtgttgagaaggattctgaggttgcatccaattttagcaggaaagagtgctgtgatcgttagctgcatcagacatgggcatggatgtaggctgtgccaacacgcatgcttcttcccagacactccttggaatcaagagaatcaatgaaccaagcagtgttcataagtctcatactttgtaccaggcactctgttaagccccatgtatccatatgtgtgctaggagggagggtaaaatataaaaaggcacaagccttggtcctgtgctcaaggagctcccagtttcaggtacaggaaagtggcatgtcatgtgttggaagtgatacctattgaatgagcatccccaaccagcagcggccctgagctgaggcagaagctatcattgtggactggggcatcagggaaggcctcctagaggagtgtaaattggggatagtttgaaaagatggatggggaggaggggtgagtaggaggcatttcaggccttggctggggagggggtttcagtggagcaaatcttttctgggatggaggccttaagaatgacagtggcttttgcctccccaaggggacacccgttccagtgagatgcccgagctcacctccatgcacaccctcttacttcgggagcacaaccggctggccacagagctcaagagcctgaaccctaggtgggatggggagaggctctaccaggaagcccggaagatcgtgggggccatggtccaggtaggccgtcttgaacaccgggcacacgggatccagatgtgtccctgcaacatcctagctgtgtgaccttgggcaagttcctaacgctcctgagccttgccttcttcatccatgaggctgtaaggattataatacatatatgaaaacacctttcagatagtaagtgcttgacaaagcctccttcccttccctctccttcttcgcaggtttgctccctgcagtcttctttcctgtgggtctgttattggtgttgggagggttgaatgtttggtttagcgcccaagtagctgttcagtaaattcttcttccctccatctcactgtcactcttagctcctttacccactcacctcttctttttaaaattaaaaaaattatagggctgggcgcggtggctcatgcctgtaatcccagcactttgggaggcggaggcgggtggatcacgaggtcaggagatcaagaccatgctggctaacacggtgaaaccccatctctactaaaaatacaaaaattagccgggcgtggtggcgggcacctgtagtcccagctacttgggaggctgaggcaggagaatggcgtgaacctgggaggcagagcttgcagtgagctgagatggcgctgctgcactccagcctgggtgacagagcgagactccatctcaaaataaataaataaataaaataaaatttaaaaattattattattattttatttttattttttgatacagggtcttaccctgttgcccaggctggagtgcagtggtgcgatcttggctcactgcaacctccacctttcaggctcaagccattctcccatctcagccttctgagtagctgggatttcaagcacgggccaccacgcccggctaatttttgtattttttgtagagacggggtttcgccacgttacccagtgtggtctcaaactcctgggctcaagtaatccgccctcctcgacctcccaaatccctgggattacaggcacgagtcactgtgttcagcccactcacctcttctttttggctctgtcataatccacccaatgtccttagcctcatttacccctggggcaatgttcctggttctgtctctcaacatctggtgcttcattgcccccaccctctccctcagaggcagaggcagacatacacacactcacacacacacacacacacacacacacacacacacactcctagattctggaggagagagttttcggtgctaagggtgagtttttgatttgcactaagaactcaagtgaaattcatttacactccaaaataacttctagactctagaatgtagagtgagactattccatgtcttcacttttcacagggaaaatcatgccagaatggcccttcgttccttcctgcctccctgctttccacaaacctaaactgagccttactgtgggcaggcacttggtgggcactgtgaagaatttcaacaacttcaatttcccttgctctcaaggactttataagatagttgcagaaatgagacattaataacttgaggacaaggaagttatcacaatgactaaaaaagtgatttgctatttagtcctctaaggcagagggcaatataattatccttaatgtacatttgaggaactcaggctcagagaggttaataattggtccaaggtcacccagccagcaagcagtagaactctatttatgcctctcagtgccactctgttaggcccaggtccggtattgtgggaggctattccctgaccatagagggatggaaggggtccccgaagccaagagcaggcagagactctggccctcctgtgctgccaggcggcatttgctgtggaggagtgggtggagaggccattccaaatgacttgtctttagatcatcacttaccgggactacctgcccctggtgctggggccaacggccatgaggaagtacctgcccacgtaccgttcctacaatgactcagtggacccacgcatcgccaacgtcttcaccaatgccttccgctacggccacaccctcatccaacccttcatgttccgcctggacaatcggtaccagcccatggaacccaacccccgtgtccccctcagcagggtcttttttgcctcctggagggtcgtgctggaaggtaagcaggacctaggccaggagcgagtgggctgacagctaaaggtggggtagggatcaccttggctctagggagctagggtggggagcagtctgctttcctcccatcttggagatctggtctgactctctagcctcagttcctctgactctgcttccatatctgataataaacagtggggtggggatggccctcagagggaactggctacttcctatgtgttgtggctcgctgtgacccaggaagccagggcctgggagcatcccaatcttgcaagtctgaatggcataatctcctgagacctatatgtccctggggtcatgtagtgaccggtgaggacctgctcactggggccaccaccttggcaccaggcactgcttgtgcccagccctcttctcgaatcctcctgaccctacctggacttgtccctgactccaatctgagctctgatactgagccagatacttcccctgacctggctcctctggtccccaggtggcattgaccccatcctccggggcctcatggccacccctgccaagctgaatcgtcagaaccaaattgcagtggatgagatccgggagcgattgtttgagcaggtcatgaggattgggctggacctgcctgctctgaacatgcagcgcagcagggaccacggcctcccaggtgaggggctgcaggagtctcccctgatgctcacctcccctgagctgcctcctaggtagcccatcaccccccttcccactctagggtccctgccctctccaaagcatattagaggcactttgctctcaggaagtagcctgaggccaggcccctgccttttcccggacccacctgataaatttccagctcctctcaacctcctttgcatgctgtaattctcatattaggggcatcgtcatcataatgataacccacagtctgaaaatctgtctctgtcatcattccattttgcttcattgtacgttaactctgggggaagagattattattgccttcattttactgatgaggaaactgaggcttgatgagattcagaacctggccaggttctctagccaagtcagtggcaaagctgaaatttgaagccagattagcctaacctcaaagcctgtgcctatgttatggggtagcccatgtggctgaggcttctagacacatttcacctctcatgggtcagagacagacacatcgggctgctctggagggggacaaggagatgtccctattcttggacccagcaggaactgtggcagtttagggtttgccttatggtaggagggcaagagaaagacagagagggtcccagagaggcccagttgtgccccttggtctaagccaaaggtacagggttccttgaggtgaggtccttccctccctctatgaccttggttgtgttgggaaggtgggttttctgggctgctggcgatatgtgaatcttccctgctgtctccagtgacctccccaccttgaagcagagagacctgcccatagccacctgtcccctcccccatgcaggatacaatgcctggaggcgcttctgtgggctcccgcagcctgaaactgtgggccagctgggcacggtgctgaggaacctgaaattggcgaggaaactgatggagcagtatggcacgcccaacaacatcgacatctggatgggcggcgtgtccgagcctctgaagcgcaaaggccgcgtgggcccactcctcgcctgcatcatcggtacccagttcaggaagctccgggatggtgatcggtgaggaggggcaggcgtcgtgggccgctgggtggctgtgggcccatccttgactctcttggagcccaaatttcctcctgtcagttgaaggactggagggagtcagtaattttccagtgtgttccaggaatcctccagatgccctggagtgcccctaatgtgccctgaacctgttgggggtgcagcaaggaaagacctggacccctggttgtaaagggtggaagatagaaaagtgccacccttagctacttcttcccatgggtttcactgtgccaccatgtgggtgtcagtcaccagtgggcagtcccctcccagctttcccacagctccccttagtgtcatttcctcgtggtcctgatgtcttcatgtctagttcttgcttagcctgtactgcacctttgggaaaattagaaaaaggtgcccctctctgggtgggcgaattataaaccacccccaagattctcttccactcctgtggatgcagctcctggccagggcacagggcttcaggagtgtgtgtgggcccgggtccccctccagcatggtgccctggaggccgccgggctaggggtaagggaaaggccactgggcagctgtgctttactctgcacgatgagtggagcatcacttgtgtgaaagcccctgggctgcccaagggcctggggccctcctgtgcttccaggtggcatttgttgtggctttgttatatcctgggagcagcacaagcccatcgatgccctgccagcccagaatatccttgggcacagtgtccatgggtgttccccatgcaggttttggtgggagaacgagggtgtgttcagcatgcagcagcgacaggccctggcccagatctcattgccccggatcatctgcgacaacacaggcatcaccaccgtgtctaagaacaacatcttcatgtccaactcatatccccgggactttgtcaactgcagtacacttcctgcattgaacctggcttcctggagggaagcctcctagaggccaggtaagggggtgcagcagtgaggggtatatctgggctggccagttggaaccacggagatctccttgccctagatgagcccagccctgttctgggtgcagctgagaaaatgagtgactagacgttcatttgtgtgctcatgtatgtgcgaagtatataaattggcttttcatgcgtgtgtgttgtctgaacatggggagtgtttcatgggttatgtgtatgtgccatttatgtgagtgtgtgtttgtgctgatgagaatactgagtatgtggaaggcagcagagcggactggtgaggagcacagctcaggaactagactgcctgggttccaatcctggctctgtggcttgctagctatgtgaccttgagcaaattaccctccttaaacaagagttttcttccttgtaaattacatctgtcatggtttcttggagggcccacttgtatcctctggttcttcatttattgagcacctactacatgcaaggcactgtactaggcgtgagaagcatatagaggcaagaaagagataccaagatgccatctgtgtcctggttagcagagctggaccagtggtgccttggagggataagccagctgcagctgggctgtgtggttgacttatgggcccagccagccaggctcaggccatggctcccctttttcttcctcaccctgatttcttgcttattcactgaagttctcctgaagaggaactgggcctgttgccctttctgtaccatttatttgctcccaatgtttatgataataaaggcaccgctgatggggacctcc

SEQ ID NO: 2 - protein, 745 aa, Homo sapiens
  MISC_FEATURE: MPO protein Accession Number NP_000241
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 3 - DNA, 4104 bp, Artificial Sequence
  Cas9 Cdna Cas9 DNA sequence from Addgene plasmid pL.CRISPR.EFS.GFP
  misc_feature: Synthetic
gccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 4 - protein, 1368 aa, Artificial Sequence
  Cas9 protein
  misc_feature: Synthetic
ADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - DNA, 20 bp, Artificial Sequence
  mouse gRNA-3
  misc_feature: Synthetic
ccccaacgatcagctgacca

SEQ ID NO: 6 - DNA, 20 bp, Artificial Sequence
  mouse gRNA-4
  misc_feature: Synthetic
cagcggggtgtacggcagcg

SEQ ID NO: 7 - protein, 21 aa, Artificial Sequence
  MOG35-55
  misc_feature: Synthetic
MEVGWYRSPFSRVVHLYRNGK

SEQ ID NO: 8 - DNA, 20 bp, Artificial Sequence
  mouse gRNA-2
  misc_feature: Synthetic
gcactcatgttcatgcagtg

SEQ ID NO: 9 - DNA, 20 bp, Artificial Sequence
  mouse gRNA 6
  misc_feature: Synthetic
tgcgatacttgtcattcggt

SEQ ID NO: 10 - DNA, 20 bp, Artificial Sequence
  mouse gRNA 1
  misc_feature: Synthetic
agtaaaacaggagctccgtg

SEQ ID NO: 11 - DNA, 20 bp, Artificial Sequence
  mouse gRNA 5
  misc_feature: Synthetic
acgcttccaagacaatggca

SEQ ID NO: 12 - DNA, 20 bp, Artificial Sequence
  mouse gRNA 7
  misc_feature: Synthetic
acgccatcttcatactctgc

SEQ ID NO: 13 - DNA, 20 bp, Artificial Sequence
  mouse gRNA 8
  misc_feature: Synthetic
atcaagcggagcctccaaag

SEQ ID NO: 14 - DNA, 20 bp, Artificial Sequence
  mouse gRNA 9
  misc_feature: Synthetic
agagtacctgtaacattgaa

SEQ ID NO: 15 - DNA, 10629 bp, Mus musculus
  misc_feature: MPO chromosome 11, GRCm38.p4 C57BL/6J DNA SEQ. ACCESSION NC_000077
ataacttctcagctttagctccctgaattttggttctttcaaaggatttgggacactcagtttaaccaacactttgaggggctggttgtttgtgctctccgggagaagctctgatccattcctttgtccacccctccttcttcatagacccctttctgtgcacccctaaccccagtgggggaggagagtctagtcttgggataggtccatctctttttaggtcagcccgcccccagcttttacaccccaggcataaaaacccgttgtcagtgagaggagttgacagtgtcaggtaagtggtgaagttgagctcttggggactggggactatagctggagggggtcttgggttgtacacctcttcatagaccaggcccactagggtagggctgtgtataaaggggagaggagggggcttgggaggaggagaattgtaaaacctctcttttttcaaaagctctggatacttcctgagctgaggtttttgttttttgtcttgttttgttttgttttgttttgttttgttttgttttgttttgttttgttttgtttttttaaaggtttatttatttattatatgtaagtacactgcagctgtccttagatactccagaagagagcatcagattttgttacggatggttgtgagccaccatgtggttgctgggatttgaactcgggacctttggaagagcagtcggcgctcttaaccactgagcaatctccccagcccccttgttttttgtcttgcagagatgaagctactcttggccttggcaggcctcctggcccctctggccatgcttcagacctccaatggtgccaccccaggtgatagtgccaaaaggagggtggactaagggattgggggtagggggtggggattctcagcccagcatctccccttcccctggataggcttcctttgctttggggcctgatgtagggctttggggccagccctgaccttatgatttatgctctgggcagctctcctgggggaagtggagaattcagttgtgctgagctgtatggaagaggccaagcagctagtggacagagcctacaaggagcggagagaaaggtgagggtgccaggccctgtgtggccctgggcaaaactgcttcaagtcttctggagaaagggcacacagtggggggaggggggttgtagaaaggatgagggcctacaggtgaaatcgtacctggtctgcctctacttgagtttctggctgtcagaacatcttcagtggccttgctttctgtctggacatccaggtccatgtccttttgacaaaagaaatgcactgctcctccgtggttctctccagagttctttctgcctgctcaccgcaggctggcctttgctgagtgtctctatcccccttctctggccctcccttgtctaaacagcatcaagcggagcctccaaagcggctctgccagccccacggagctcctgttttacttcaagcagccggtggcgggcaccagaacagctgtgagggccgctgattatctacatgtggccctagacctgctgaagaggaagctgcagcccctgtggccaaggcctttcaatgttacaggtactctaactccttttgtgctaccacctccaccccaagcaacttctgtctccaagcctccattcaaggcagtggttcttaacctgtgggtcgctaccccttcgggtacgtcaagtgaccctctcacatgggtccacctaagaccataggaaaacacaggtcttaacattatgattcatctcagtagcaaaattacagttatgaagtagcaagtaattttatagttctgttcaccccatgagaaactggcttaaggggtctctcagcattagcaaacttgagaaccactggtctaaggagtctcggggcctctgagctggcccacccccctcctcaccctcactgggctactcagccccaggttgcctagcataggaaatgaggtgggggaaggctcggatctaagatcatggctcactcttgcagatgtgttgacacctgctcagctgaatctgttgtccgtgtcaagtggctgtgcctatcaggacgtgagggtgacatgcccaccgaatgacaagtatcgcaccatcactggacactgcaacaacaggtgtgggcccagggcagttcagggttgtgtggtgtagggggtctaggtcttgcctgtggcgacccattgggccagagctctgtgcttccctctagacgaagccccactctgggggcctccaaccgtgcctttgtacgctggttgcctgcagagtatgaagatggcgtctccatgccctttggctggacgcctggagtcaatcgcaatggcttcaaggtgcccctggtgagtgtccagtagagggaggtgagcccagctaagcttatgtgcaggacctaggcatccaatgactcctgttgttgctgcaggctcgccaggtctccaatgccatcgtgcgcttccccaacgatcagctgaccaaggaccaggagcgtgcactcatgttcatgcagtggggacagtttctggatcatgatatcaccttgactccagagccagctacccggttctccttcttcactggcctcaactgcgagaccagctgcctgcagcagccaccctgcttccccctcaaggtgctgcctccttccatccatcagggtctgtgtctgaagagagggaagatggtttacattagtgacggcgccccttctgagtcctaggtcctctctagcccctgagcaagactggtcctcgctcctgggcccagagtcaatacttggctccctcagcagagccagctgtcaactcaaggaacctgatatgcaccctgatatgcaccctgaacttagcatcgctctgtgtggtggggtgactggttctttgtgaggctgggagaattctgggatgaggatctgacttgtcttcttcatactcccaatgtacaggcactggcctgttctcactctctctcccctatgggcgctcccctaaccctgcttcacagtgtgtgtgtgtgtgtgtgtgtgtgtgtaaccctgcttcacagtgtgtgtgtgtgtgtgtgtgtgtgtgtgtgtgtgtgtgtgtgtgtgtgtgtgtgtgtgtttgggttgccccatagtcagggtcagggttgaagcaccttctgtgtaggggggaaagtgtggagaaaaagaaaggcagagattaaggagataaagggccaggggtctgacagggccagggtgacagaacagagaggaaaaggcagacacaggcaagaagaggattaagaggcatagtgaagaggggtccaagtggccttgcccaaccccagaaggctctctccattccagttcagtgaattctttgtcacccttaaacctcagtctctttgctgactgagccccagtatttctatgaaaccttgagagcttccctgcagctcgagggggccagagttctcttctgcagggtgtaagttacaaatgcccgctctcccgacccacaaaagccagcctagcttctctctgtgctgtagatcccacccaatgaccctcgaatcaagaaccaaaaggactgcatccccttcttccgctcctgcccggcatgcaccaggaacaacatcaccattcgcaaccagatcaacgcgctcacttccttcgtggacgccagcggggtgtacggcagcgaggaccccctagccagaaagctgcgcaacctcaccaaccagctggggctgctggctatcaatacacgcttccaagacaatggcagggccctgatgccctttgacagcctgcacgatgacccctgcctcctcaccaaccgctccgcccgcattccttgttttctggcaggtcagcccggggactgaactagagtggcatagagctgtttccagcttgtgagaacacagcctgtatgtgagcaacatgcgggcttgtgataagagatgctggtctcaacaacttattagtctagttgccttggtaacgagttggatggagacagaaaggaaaataacagcaaaaatcaaaccacataagcatacaaaaaaaaaatcctccatccatcaactgaagatggagtcacagggctgctaaggaccctctattattgatccagctttctctgtcttctccctcctgtggactgtggaaaaagagagagccaagtctatgtccacaagggacagagaactaaaagtgacagagaggaatatggcatcctcaccttttagaaccatcctgtccaatctggagctagtagccacatgtggttatttcaatttaaattctttgtagcagggagtactggctcacacctttaatcccagtctttgggaggtagaggcatgaggctcttagtgagaattcaaggccagcctggtctacagagtgagttctaggacagccagggctatagagtgatagtgagaccctgtcttgaaagaagaagaagaagaagaagaagaagaagaagaagaagaagaagaagaagaagaagaagaagaagaagaagaagaagaagaagaagaagaagaagaaaacaaccaataaatacataaataattttaaattaaattaaaagtttctgcagtaactgcattttaagggctcagtagtccatgggtgaagaagtggttgttttcttcacctcagaaaattctagtagattgtgtcacagaatatcactccgtgagctttttggtttcttagaaagacagaaggtgggatttcaaaaggagagatgtttggcagtttggggataggattggggcaggggttgtggtcacacccagaaattgatgcctctccctcacacctctctgtgattttagactgggttatagctagccttcgcaggtctggtacttgctttgaagcccaggctagcttcaaacattcctcaatcttcctatcttagcctgtcaagtgttgggtggctcacatgtaccatcaagcccacacacagcgggaggtctcttcttgcacctcccttcccttctaggggtccagttggtctctcctgggtcccctggcagggcagccaaggtcaatgcaagggtgtgtctcctaagggagggcctgctctcttctggactggtcatgacctctggttactcatggggtggaaaagctactctcaggaatatgttttcctctcagaattttctttctaagttggcaagggctgagcccagtcagtgtcttgcagcaaaggccctggcccccaccttctggccttccctgttgaaggtttatggagcactagaggaatcggggacttcattttcttgaatagtcttaggtatggatgtacacattccgtgccctcttcccaccatgacaggatgtacagtggcttatagtgagagtgataatgacaactgtcttcaaatagtagagggtttggggtcctgttttgagaaggcttctgaggttgatccaatttgagcagaaaacatgcatattactttttggcttgtgccaatgagtacttgctcttgggagtgttcatgtattgtggagtgtgtgtgtatgtgtgtgtagtatgtatgtatgtatgtatgcatgcatgcatataagtatgtgtagtatgtgtgtgtaatatgtctgtatgtctgtgtagtgtgtatgcatatgtatatatgtgtgtatgtatagtatgtgtatgtagtatgtgtatatgttattatgagtgtgtatagtatgtatgtgtatagtgtatgtgcatgcatatataatgtatatgtatgtgtgcatatgtgtgtagtatgtgtacatgtgtgtgtgtagtaaatggcaaaatacagaaagacacagggcttggccttgtgcctaaggatcttgtgcctaaggtctgctccaggcaggtgacaagtcaaatgtaatggctattgggtaaggctgcagggagtaccacagtgttcctgagctcttatgccagcctgtggctcacacttgaatctggtgtgtgggagtaggtggaaaacaggcacgtgagggccactgaagctctttgctggtcaggagatgatggagaccataaaggtgacaaaggctttgcttccctcaggggacatgcgctccagcgagatgccggagctcacctccatgcacaccctctttgttcgagagcataaccggctggccacacagctcaagcgcctgaatcctcgatggaatggggagaagctctaccaggaggcccggaagattgtaggggccatggtccaggtaggctgtgtcctaacacatcttggccatgtgttcttgggcagtttttttttttaaatctctcctgaacactggcttttttttatccataaaaggctcaaaggagcgaaatgaaaatgcccctcagacatgagatgcttggcgagtgcacttcccttcctcctgtctccccggccctcttccttcctgagggcctgaggagttgagagggtcttaagcagaagcaccagagtcaccgctggctcttttacccgctgtcatcatcttgagcattcccccacccccacccccacccccaccccacccctgccacccggggcagggctcctccatctggtttttgatgttttcatgacccctaatttcccaagaatgtaaacattcgtgcatatcacacacacacacacacacaacacacacacacacacacacacacacatacacacactccctagactttagggagggggtgtggttagtgctgagcatggggttttgattgacaggcacacccacaagagggattcactttggtctcagaggcaattctagccttaggtccagagtgacactgtctcacagcctttgggggacatcgcactggagcagacctctgccatctctctgtctataaacctgtactgagccctactgtgtgctagtctattgctggacactgtgaataatctcagtaatttcaacttccctgggtctcaaggggaaatgggaccactaataccttaaagacacgggggatattacaatgactaaaaagacattttgtttaatcctgagagagagggagagagaattattccttgtttacatttgtggttcagagagtttaataaatgacctgaggtgagttctatttatgtccttctgttaaaatctaagacctaagactatgggagactgatcctagccaggtaggggcaattcccccaagctggagaccacagggaggaacaaatagacatctaacacaaagtgactttgttttagatcatcacataccgggactacctgcccttggtgttggggccagcagccatgaagaagtacctaccccagtaccgatcttacaacgactcagtagaccctcgaatcgccaatgtcttcaccaacgctttccgttatggccacaccctcatccaacccttcatgttccgcctgaacaatcagtaccggcccacagggcccaacccccgagtcccactcagcaaggtcttttttgccagctggagagtcgtgttggaaggtgaatggaacctaagaaaagccagcagctaatgatgggatggtggctgcagagacctaggctagggagaggtcttttttcatcttggggaacccagtcttgctttccactttcattgacttagaaggctagcgcatcccaaccttgtgaaccctcccccaaccctccaagtgttgcaagagcttatagcttctctctgacccctacgattccacttggaccagccctgactccactgtgtgctctcatagtaagagaaccaacctttcctttcactctggtctcttgtcctcaggtggcattgaccccatcctccgaggcctcatggccactccagccaaactgaatcgccagaatcaaattgtggtggatgagatccgggagcgactatttgagcaagtcatgaggataggactggatttgcctgctcttaacatgcagcgcagccgggatcacggcctcccaggtgagaggccagccacagccccatcccacctcccaggcagctccccttcttgctcaggggtcctttccagttttatggatgcacctgaggcaaggcgctgccttttccagcccctggtggcctaagttcggcctctcttggcttcttcacaagctggacttttgcaacccgcgtagcagctgaacatcatcccacgatctggaagtctgcctccatcgttagcacgttctgactcattcatgaactcgcgggtgggagagtaccactgtcatttttactgacgaggaaactagggcttagagcagttcagagtctggctcggtgtggttgccacgtcagtgagagacgtggaggttggacctaaggggatcagcggaacccaaaagccccgtgcctgtgttgtggagtagcacaagtggccacgcctctgggaaacgttttcattctctgagtcagagaaagatacattaggctgctgctctaagggacaatgtgctgtttccgtgcttgagtctggcgggatttgtggtaatacagggctggccagggtaggagagagtgaggaaaagagtgagaggtctggaggagcccagctgcctactttggcctgagccaatcatttagggatccattctacttacggtcttgtgtactgagatgggcttttgaacatctgtgtattctttcctgcccttcccagtactcctagccggcaggagaggggtccatttacagctacctgttctctggcccaacgcaggatacaatgcctggagacgcttttgtgggcttccacagcccagcacagtgggtgagctcggcacggtgctgaagaacctggagttggcacggaagctgatggcacaatatggcacgcccaacaacattgacatctggatgggcggtgtgtccgagcccctggagcccaatggccgtgtaggccagctccttgcctgcctcattggcactcagtttaggaagctacgtgatggtgatcggtaaggaagctgtgggaaagcagggtgccacaactgtgaggccgggagggggcgcatgcgcaccgcagcagtcaggcggcgcttagagaacaacttatctgtgagttggttccatcatgtctggtctagggatggtagtcaagttctcagccttggtggtacgtgcctttgcccactgaaccatcttgctggtcccaaaaatgccactcttaataggttctctgggtttctcagtgctactgtggaggcgtcagttaccatggaaaagtgccaaaagtctcccgctgaccccttttcatgtcatttcatcacgatcttggaatctccgtgtctagttcttatctagcttgcattacacctttggcaatatcgtaaaaaggtgtcccagtcaagaagagaacatcacaaatgccacccaggctttcttttaccccgtgggagcgtgcatcagactgggacccaagcagctgtgcttctgtcttctgggctgagtgggcactgcccacgacctgagctctcctgtgctgtagataagaatattctgaagccccctcacactcaggggacgtctcaagccaggggatgtcctgggtagcctcactcgggacatcctcagacataatgtccacgatgctccctcccatgcaggttttggtgggagaacccaggcgtgttcagtaaacagcagagacaggccttggccagcatctccttgccccgtatcatctgtgacaacactggcatcaccaccgtgtcgaagaacaacatcttcatgtccaacacatacccccgagactttgtcagctgtaacacccttcctaaactgaacctgacttcctggaaggagacctagaggttgggtaagggcttggcacactgaagggtatacctggttggctagtgggaatcacagagaactctcttccctgggtgaatccatctttgtggatattgtgggtgacagtggggggtggggggaaatgactggacattcacttgtgagtgcacaagtatatttgcatatacacttgtgcacatctatatatatgcaacatacataaactagtatttcatgtgtgtctggtctgtgagcatgggcaatgtttgatgtgccttgtgtgcctgccaatcacatgagtctgggctcactgcttacgacaatattaggaacgtggggacagtgagcagacagcccgagttcaagtcatcaccctgtagcacagtactgtgtgtgactttggccaaattactctaaggataaattctcttccttataaatgaaatcagccacggcttgttagagggcctattgcctcctccaactctccactcatggagcagctcccatgtgctaggcactgtgctagccatgggaagcagaaacaataaagatgaataccaaagatgaatgccaaatcctcgatcaccttctggttggcagaaacaaggtggtggcctccggaggagaggatgcagctagcagctgggctgtgtggttgtgatacagttctgggccagctctgcttaactggctatttctccttttcccttactctcttctcttgctattcacaggagtctgccgggagaaggacggaggccattggccttcctgtgctatttgtacctgaggttcacactaataaaacatggtggatgggga

SEQ ID NO: 16 - protein, 718 aa, Mus musculus
  MISC_FEATURE: MPO PROTEIN SEQ. ACCESSION NP_034954
MKLLLALAGLLAPLAMLQTSNGATPALLGEVENSVVLSCMEEAKQLVDRAYKERRESIKRSLQSGSASPTELLFYFKQPVAGTRTAVRAADYLHVALDLLKRKLQPLWPRPFNVTDVLTPAQLNLLSVSSGCAYQDVRVTCPPNDKYRTITGHCNNRRSPTLGASNRAFVRWLPAEYEDGVSMPFGWTPGVNRNGFKVPLARQVSNAIVRFPNDQLTKDQERALMFMQWGQFLDHDITLTPEPATRFSFFTGLNCETSCLQQPPCFPLKIPPNDPRIKNQKDCIPFFRSCPACTRNNITIRNQINALTSFVDASGVYGSEDPLARKLRNLTNQLGLLAINTRFQDNGRALMPFDSLHDDPCLLTNRSARIPCFLAGDMRSSEMPELTSMHTLFVREHNRLATQLKRLNPRWNGEKLYQEARKIVGAMVQIITYRDYLPLVLGPAAMKKYLPQYRSYNDSVDPRIANVFTNAFRYGHTLIQPFMFRLNNQYRPTGPNPRVPLSKVFFASWRVVLEGGIDPILRGLMATPAKLNRQNQIVVDEIRERLFEQVMRIGLDLPALNMQRSRDHGLPGYNAWRRFCGLPQPSTVGELGTVLKNLELARKLMAQYGTPNNIDIWMGGVSEPLEPNGRVGQLLACLIGTQFRKLRDGDRFWWENPGVFSKQQRQALASISLPRIICDNTGITTVSKNNIFMSNTYPRDFVSCNTLPKLNLTSWKET

SEQ ID NO: 17 - DNA, 20 bp, Artificial Sequence
  Primer APOE Forward
  misc_feature: Synthetic
gagctgatctgtcacctccg

SEQ ID NO: 18 - DNA, 19 bp, Artificial Sequence
  Primer APOE Reverse
  misc_feature: Synthetic
gacttgtttcggaaggagc

SEQ ID NO: 19 - DNA, 20 bp, Artificial Sequence
  CCL2 Forward
  misc_feature: Synthetic
gggatcatcttgctggtgaa

SEQ ID NO: 20 - DNA, 20 bp, Artificial Sequence
  CCL2 Reverse
  misc_feature: Synthetic
aggtccctgtcatgcttctg

SEQ ID NO: 21 - DNA, 21 bp, Artificial Sequence
  CXCL10 Forward
aactgcatccatatcgatgac

SEQ ID NO: 22 - DNA, 19 bp, Artificial Sequence
  CXCL10 Reverse
  misc_feature: Synthetic
gtggcaatgatctcaacac

SEQ ID NO: 23 - DNA, 24 bp, Artificial Sequence
  IL1beta Forward
  misc_feature: Synthetic
ggagaaccaagcaacgacaaaata

SEQ ID NO: 24 - DNA, 23 bp, Artificial Sequence
  IL1beta Reverse
  misc_feature: Synthetic
tggggaactctgcagactcaaac

SEQ ID NO: 25 - DNA, 20 bp, Artificial Sequence
  MPO Forward
  misc_feature: Synthetic
tggtggcctgcagagtatga

SEQ ID NO: 26 - DNA, 20 bp, Artificial Sequence
  MPO Reverse
  misc_feature: Synthetic
gttgaggccagtgaagaagg

SEQ ID NO: 27 - DNA, 22 bp, Artificial Sequence
  S100beta Forward
  misc_feature: Synthetic
ttgccctcattgatgtcttcca

SEQ ID NO: 28 - DNA, 24 bp, Artificial Sequence
  S100beta Reverse
  misc_feature: Synthetic
tctgccttgattcttacaggtgac

SEQ ID NO: 29 - DNA, 20 bp, Artificial Sequence
  TNFalpha Forward
  misc_feature: Synthetic
agggtctgggccatagaact

SEQ ID NO: 30 - DNA, 21 bp, Artificial Sequence
  TNFalpha Reverse
  misc_feature: Synthetic
ccaccacgctcttctgtctac

SEQ ID NO: 31 - DNA, 23 bp, Artificial Sequence
  VCAM1 Forward
  misc_feature: Synthetic
ggagcctgtcagttttgagaatg

SEQ ID NO: 32 - DNA, 23 bp, Artificial Sequence
  VCAM1 Reverse
  misc_feature: Synthetic
ttggggaaagagtagatgtccac

SEQ ID NO: 33 - DNA, 20 bp, Artificial Sequence
  T173
  misc_feature: Synthetic
tgcacatcccggtgatggtg

SEQ ID NO: 34 - DNA, 20 bp, Artificial Sequence
  D260
  misc_feature: Synthetic
caggggtgaagtcgaggtcg

SEQ ID NO: 35 - DNA, 20 bp, Artificial Sequence
  H502
  misc_feature: Synthetic
ggatgagggtgtggccgtag

SEQ ID NO: 36 - DNA, 20 bp, Artificial Sequence
  T173 T7 Fw primer
  misc_feature: Synthetic
tcttcctccagggagtctca

SEQ ID NO: 37 - DNA, 20 bp, Artificial Sequence
  T173 T7 Rv primer
  misc_feature: Synthetic
gcaggaaggagacaggtcat

SEQ ID NO: 38 - DNA, 20 bp, Artificial Sequence
  C319 T7 Fw primer
  misc_feature: Synthetic
ccaggttcccagttcagtgt

SEQ ID NO: 39 - DNA, 20 bp, Artificial Sequence
  C319 T7 Rv primer
  misc_feature: Synthetic
ccacagcgttctctgtcctt

SEQ ID NO: 40 - DNA, 20 bp, Artificial Sequence
  H502 T7 Fw primer
  misc_feature: Synthetic
atgcctctcagtgccactct

SEQ ID NO: 41 - DNA, 20 bp, Artificial Sequence
  H502 T7 Rv primer
  misc_feature: Synthetic
agccaggtcaggggaagtat

SEQ ID NO: 42 - DNA, 20 bp, Artificial Sequence
  C319 gRNA
  misc_feature: Synthetic
ggatggtgatgttgctcccg

SEQ ID NO: 43 - DNA, 23 bp, Artificial Sequence
  T173 protospacer
  misc_feature: Synthetic
tgcacatcccggtgatggtgcgg

SEQ ID NO: 44 - DNA, 23 bp, Artificial Sequence
  D260 protospacer
  misc_feature: Synthetic
caggggtgaagtcgaggtcgtgg

SEQ ID NO: 45 - DNA, 23 bp, Artificial Sequence
  H502 protospacer
  misc_feature: Synthetic
ggatgagggtgtggccgtagcgg

SEQ ID NO: 46 - DNA, 23 bp, Artificial Sequence
  C319 protospacer
  misc_feature: Synthetic
ggatggtgatgttgctcccgggg

SEQ ID NO: 47 - DNA, 20 bp, Artificial Sequence
  C167A-1 gRNA
  misc_feature: Synthetic
ggacgtgggggtgacttgcc

SEQ ID NO: 48 - DNA, 36 bp, Artificial Sequence
  C167A Before and after cut
  misc_feature: Synthetic
caggacgtgggggtgacttgcccggagcaggacaaa

SEQ ID NO: 49 - DNA, 103 bp, Artificial Sequence
  Template ssODN oligo for C167A
  misc_feature: Synthetic
gttgtccaagtcaagcggctgcgcctaccaggacgtgggggtgactgcccctgagcaggacaaataccgcaccatcaccgggatgtgcaacaacaggtgcggc

SEQ ID NO: 50 - DNA, 20 bp, Artificial Sequence
  C180A-1 gRNA
  misc_feature: Synthetic
tcaccgggatgtgcaacaac

SEQ ID NO: 51 - DNA, 39 bp, Artificial Sequence
  C180A-1 Before and after cut
  misc_feature: Synthetic
accatcaccgggatgtgcaacaacaggtgcggctggctg

SEQ ID NO: 52 - DNA, 103 bp, Artificial Sequence
  Template ssODN oligo for C180A
  misc_feature: Synthetic
cttgcccggagcaggacaaataccgcaccatcaccgggatggccaacaacagctgcggctggctgggggtggctgcaggaaccgggctcagagaggcgtcccg

SEQ ID NO: 53 - DNA, 36 bp, Artificial Sequence
  Y173C -1 Before and after cut
  misc_feature: Synthetic
tgcccggagcaggacaaataccgcaccatcaccggg

SEQ ID NO: 54 - DNA, 103 bp, Artificial Sequence
  Template ssODN oligo for Y173C
  misc_feature: Synthetic
ggctgcgcctaccaggacgtgggggtgacttgcccggagcaggacaaatgtcgcaccatcaccgggatgtgcaacaacaggtgcggctggctgggggtggctg

SEQ ID NO: 55 - DNA, 20 bp, Artificial Sequence
  gRNA Q257A-1
  misc_feature: Synthetic
catgcaatggggccagctgt

SEQ ID NO: 56 - DNA, 36 bp, Artificial Sequence
  Q257A -1 Before and after cut
  misc_feature: Synthetic
catgttcatgcaatggggccagctgttggaccacga

SEQ ID NO: 57 - DNA, 103 bp, Artificial Sequence
  template ssODN oligo for Q257A
  misc_feature: Synthetic
gactccggaccaggagcgctcactcatgttcatgcaatggggcgcgctgtttgaccacgacctcgacttcacccctgagccggccgcccgggcctccttcgtc

SEQ ID NO: 58 - DNA, 43 bp, Artificial Sequence
  D260A-1 Before and after cut
  misc_feature: Synthetic
gcaatggggccagctgttggaccacgacctcgacttcacccct

SEQ ID NO: 59 - DNA, 103 bp, Artificial Sequence
  template ssODN oligo for D260A
  misc_feature: Synthetic
ccggaccaggagcgctcactcatgttcatgcaatggggccagctgttggcacacgacctcgacttcacccctgagccggccgcccgggcctccttcgtcactg

SEQ ID NO: 60 - DNA, 20 bp, Artificial Sequence
  gRNA M409A -1
  misc_feature: Synthetic
ggtgagctcgggcatctcac

SEQ ID NO: 61 - DNA, 41 bp, Artificial Sequence
  M409A -1 Before and after cut
  misc_feature: Synthetic
aggggacacccgttccagtgagatgcccgagctcacctcca

SEQ ID NO: 62 - DNA, 103 bp, Artificial Sequence
  template ssODN oligo for M409A
  misc_feature: Synthetic
gaggccttaagaatgacagtggcttttgcctccccaaggggacacccgttacagtgaggcgcccgagctcacctccatgcacaccctcttacttcgggagcac

SEQ ID NO: 63 - DNA, 20 bp, Artificial Sequence
  gRNA E408A -1
  misc_feature: Synthetic
ggtgagctcgggcatctcac

SEQ ID NO: 64 - DNA, 41 bp, Artificial Sequence; identical to SEQ ID NO: 61
  E408A -1 Before and after cut
  misc_feature: Synthetic

SEQ ID NO: 65 - DNA, 103 bp, Artificial Sequence
  template ssODN oligo for E408A
  misc_feature: Synthetic
gaggccttaagaatgacagtggcttttgcctccccaaggggacacccgttacagtgcgatgcccgagctcacctccatgcacaccctcttacttcgggagcac

SEQ ID NO: 66 - DNA, 20 bp, Artificial Sequence
  gRNA H261A -1
  misc_feature: Synthetic
caggggtgaagtcgaggtcg

SEQ ID NO: 67 - DNA, 39 bp, Artificial Sequence
  H261A -1 Before and after cut
  misc_feature: Synthetic
gcagcgcagcagggaccacgacctcccaggtgaggggct

SEQ ID NO: 68 - DNA, 104 bp, Artificial Sequence
  template ssODN oligo for H261A
  misc_feature: Synthetic
atgaggattgggctggacctgcctgctctgaacatgcagcgcagcagggacgccgacctcccaggtgaggggctgcaggagtctcccctgatgctcacctcccc

SEQ ID NO: 69 - DNA, 39 bp, Artificial Sequence
  C319A -1 Before and after cut
  misc_feature: Synthetic
cctggcccagatctcattgccccggatcatctgcgacaa

SEQ ID NO: 70 - DNA, 102 bp, Artificial Sequence
  template ssODN oligo for C319A
  misc_feature: Synthetic
gggtgtgttcagcatgcagcagcgacaggccctggcccagatctcatgccaccggatcatctgcgacaacacaggcatcaccaccgtgtctaagaacaacat

SEQ ID NO: 71 - DNA, 20 bp, Artificial Sequence
  gRNA M251A -1
  misc_feature: Synthetic
gctcactcatgttcatgcaa

SEQ ID NO: 72 - DNA, 39 bp, Artificial Sequence
  M251A Before and after  cut
  misc_feature: Synthetic
gagcgctcactcatgttcatgcaatggggccagctgttg

SEQ ID NO: 73 - DNA, 103 bp, Artificial Sequence
  template ssODN oligo for M251A
  misc_feature: Synthetic
ccactgatcagctgactccggaccaggagcgctcactcacgttcatgcaatgtggccagctgttggaccacgacctcgacttcacccctgagccggccgcccg

SEQ ID NO: 74 - DNA, 20 bp, Artificial Sequence
  gRNA R569W -1
  misc_feature: Synthetic
aattgcagtggatgagatcc

SEQ ID NO: 75 - DNA, 37 bp, Artificial Sequence
  R569W -1 Before and after cut
  misc_feature: Synthetic
gcagtggatgagatccgggagcgattgtttgagcagg

SEQ ID NO: 76 - DNA, 103 bp, Artificial Sequence
  template ssODN oligo for R569W
  misc_feature: Synthetic
cacccctgccaagctgaatcgtcagaaccaaattgcagtggatgagatctgagagcgattgtttgagcaggtcatgaggattgggctggacctgcctgctctg

SEQ ID NO: 77 - DNA, 20 bp, Artificial Sequence
  gRNA R499C-1
  misc_feature: Synthetic
ttcaccaatgccttccgcta

SEQ ID NO: 78 - DNA, 33 bp, Artificial Sequence
  R499C-1 Before and after cut
  misc_feature: Synthetic
accaatgccttccgctacggccacaccctcatc

SEQ ID NO: 79 - DNA, 103 bp, Artificial Sequence
  template ssODN oligo for R499C
  misc_feature: Synthetic
gactcagtggacccacgcatcgccaacgtcttcaccaatgccttctgctaccgccacaccctcatccaacccttcatgttccgcctggacaatcggtaccagc

SEQ ID NO: 80 - DNA, 20 bp, Artificial Sequence
  gRNA G501S-1
  misc_feature: Synthetic
ttcaccaatgccttccgcta

SEQ ID NO: 81 - DNA, 33 bp, Artificial Sequence; identical to SEQ ID NO: 78
  G501S-1 Before and after cut
  misc_feature: Synthetic

SEQ ID NO: 82 - DNA, 104 bp, Artificial Sequence
  template ssODN oligo for G501S
  misc_feature: Synthetic
gactcagtggacccacgcatcgccaacgtcttcaccaatgccttccgctacagccacaccctcatccaacccttcatgttccgcctggacaatcggtaccagcc

SEQ ID NO: 83 - DNA, 35 bp, Artificial Sequence
  H502A-1 Before and after cut
  misc_feature: Synthetic
accaatgccttccgctacggccacaccctcatcca

SEQ ID NO: 84 - DNA, 103 bp, Artificial Sequence
  template ssODN oligo for H502A
  misc_feature: Synthetic
tacaatgactcagtggacccacgcatcgccaacgtcttcaccaatgccttacgctacggcgccaccctcatccaacccttcatgttccgcctggacaatcggt

SEQ ID NO: 85 - DNA, 20 bp, Artificial Sequence
  gRNA C158A-1
  misc_feature: Synthetic
gtcaagcggctgcgcctacc

SEQ ID NO: 86 - DNA, 37 bp, Artificial Sequence
  C158A-1 Before and after cut
  misc_feature: Synthetic
gtccaagtcaagcggctgcgcctaccaggacgtgggg

SEQ ID NO: 87 - DNA, 103 bp, Artificial Sequence
  template ssODN oligo for C158A
  misc_feature: Synthetic
gacgcccgcccagctgaatgtgttgtccaagtcaagcggcgccgcctaccatgacgtgggggtgacttgcccggagcaggacaaataccgcaccatcaccggg

SEQ ID NO: 88 - DNA, 20 bp, Artificial Sequence
  gRNA N355A-1
  misc_feature: Synthetic
gctggttggacatgttgcgc

SEQ ID NO: 89 - DNA, 38 bp, Artificial Sequence
  N355A-1 Before and after cut
  misc_feature: Synthetic
ccctggccaggaacctgcgcaacatgtccaaccagctg

SEQ ID NO: 90 - DNA, 103 bp, Artificial Sequence
  template ssODN oligo for N355A
  misc_feature: Synthetic
ttcgtggacgccagcatggtgtacggcagcgaggagcccctggccaggaatctgcgcgccatgtccaaccagctggggctgctggccgtcaaccagcgcttcc

SEQ ID NO: 91 - DNA, 20 bp, Artificial Sequence
  gRNA R128A-1
  misc_feature: Synthetic
tggctctagacctgctggag

SEQ ID NO: 92 - DNA, 32 bp, Artificial Sequence
  R128A-1Before and after cut
  misc_feature: Synthetic
tagacctgctggagaggaagctgcggtccctg

SEQ ID NO: 93 - DNA, 103 bp, Artificial Sequence
  template ssODN oligo for R128A
  misc_feature: Synthetic
cggcggtgagggccgctgactacctgcacgtggctctagacctgctggaggcgaagctgcggtccctgtggcgaaggccattcaatgtcactgatgtgctgac

SEQ ID NO: 94 - DNA, 23 bp, Artificial Sequence
  C167A-1 protospacer
  misc_feature: Synthetic
ggacgtgggggtgacttgcccgg

SEQ ID NO: 95 - DNA, 23 bp, Artificial Sequence
  C180A-1 protospacer
  misc_feature: Synthetic
tcaccgggatgtgcaacaacagg

SEQ ID NO: 96 - DNA, 23 bp, Artificial Sequence
  Q257A -1 protospacer
  misc_feature: Synthetic
catgcaatggggccagctgttgg

SEQ ID NO: 97 - DNA, 23 bp, Artificial Sequence
  M409A -1 protospacer
ggtgagctcgggcatctcactgg

SEQ ID NO: 98 - DNA, 23 bp, Artificial Sequence
  E408A -1 protospacer
  misc_feature: Synthetic
ggtgagctcgggcatctcactgg

SEQ ID NO: 99 - DNA, 23 bp, Artificial Sequence
  H261A -1 protospacer
  misc_feature: Synthetic
caggggtgaagtcgaggtcgtgg

SEQ ID NO: 100 - DNA, 23 bp, Artificial Sequence
  M251A -1 protospacer
  misc_feature: Synthetic
gctcactcatgttcatgcaatgg

SEQ ID NO: 101 - DNA, 23 bp, Artificial Sequence
  R569W -1 protospacer
  misc_feature: Synthetic
aattgcagtggatgagatccggg

SEQ ID NO: 102 - DNA, 23 bp, Artificial Sequence
  R499C-1 protospacer
  misc_feature: Synthetic
ttcaccaatgccttccgctacgg

SEQ ID NO: 103 - DNA, 23 bp, Artificial Sequence
  G501S-1 protospacer
  misc_feature: Synthetic
ttcaccaatgccttccgctacgg

SEQ ID NO: 104 - DNA, 23 bp, Artificial Sequence
  C158A-1 protospacer
  misc_feature: Synthetic
gtcaagcggctgcgcctaccagg

SEQ ID NO: 105 - DNA, 23 bp, Artificial Sequence
  355A-1 protospacer
  misc_feature: Synthetic
gctggttggacatgttgcgcagg

SEQ ID NO: 106 - DNA, 23 bp, Artificial Sequence
  R128A-1 protospacer
  misc_feature: Synthetic
tggctctagacctgctggagagg

(19) United States
(12) Patent Application Publication
(10) Pub. No.: US 2021/0121539 A1
(43) Pub. Date: Apr. 29, 2021

(54) METHODS AND COMPOSITIONS FOR MODULATING MYELOPEROXIDASE (MPO) EXPRESSION

(71) Applicants: LEMPO THERAPEUTICS LTD, Binyamina (IL); RAMOT AT TEL-AVIV UNIVERSITY LTD., Tel Aviv (IL)

(72) Inventors: Anat KAHANA, Rosh Haayin (IL); Rotem VOLKMAN, Tel Aviv (IL); Ben Zion GARTY, Tel Aviv (IL); Daniel OFFEN, Kfar HaRoe (IL)

(21) Appl. No.: 17/257,764

(22) PCT Filed: Jul. 4, 2019
(86) PCT No.: PCT/IL2019/050745

§ 371 Date: Jan. 4, 2021

(30) Foreign Application Priority Data
Jul. 5, 2018 (IL) 260445

(51) Int. Cl. A61K 38/46; A61K 35/28; A61K 31/7105; C12N 15/90; A61P 37/06; A61P 25/28

(52) U.S. Cl. CPC A61K 38/465; A61K 35/28; A61K 31/7105; C12N 15/907; C12N 2510/00; A61P 25/28; C12N 2310/20; C12N 2800/80; A61P 37/06

(57) ABSTRACT

The present invention relates to methods of modulating the expression and/or activity of Myeloperoxidase (MPO) in a mammalian subject, by modulating ex vivo and/or in vivo MPO levels and/or activity in undifferentiated bone marrow (BM) cells of the subject. The invention further provide therapeutic methods and compositions for treating MPO-related disorders.

### FIELD OF THE INVENTION

The invention relates to methods and compositions for modulating Myeloperoxidase (MPO) expression, and uses thereof in the treatment of MPO-related conditions.

### BACKGROUND ART

References considered relevant as background to the presently disclosed subject matter are listed below:
[1] Nauseef W M, Borregaard N. Neutrophils at work. Nat Immunol. 2014; 15:602-611.
[2] Delgado-Rizo V, Martínez-Guzmán M A, Iñiguez-Gutierrez L, García-Orozco A, Alvarado-Navarro A, Fafutis-Morris M. Neutrophil extracellular traps and its implications in inflammation: An overview. Front Immunol. 2017; 8.
[3] Winterbourn C C, Kettle A J, Hampton M B. Reactive Oxygen Species and Neutrophil Function. Annu Rev Biochem. 2016; 85:765-792.
[4] Metzler K D, Goosmann C, Lubojemska A, Zychlinsky A, Papayannopoulos V.
Myeloperoxidase-containing complex regulates neutrophil elastase release and actin dynamics during NETosis. Cell Rep. 2014; 8:883-896.
[5] Strzepaa A, Pritchardc K A, Dittel B M. Myeloperoxidase: A new player in autoimmunity. Cell Immunol. 2017; 317: 1-8.
[6] Khan A A, Alsahli M A, Rahmani A H. Myeloperoxidase as an Active Disease Biomarker: Recent Biochemical and Pathological Perspectives. Med. Sci. 2018 633.
[7] Zenaro E, Pietronigro E, Bianca V Della, et al. Neutrophils promote Alzheimer's disease-like pathology and cognitive decline via LFA-1 integrin. Nat Med. 2015; 21(8):880-886.
[8] Azevedo E P C, Guimarães-Costa A B, Torezani G S, et al. Amyloid fibrils trigger the release of neutrophil extracellular traps (NETs), causing fibril fragmentation by NET-associated elastase. J Biol Chem. 2012; 287(44):37206-37218.
[9] Gellhaar S, Sunnemark D, Eriksson H, Olson L, Galter D. Myeloperoxidase-immunoreactive cells are significantly increased in brain areas affected by neurodegeneration in Parkinson's and Alzheimer's disease. Cell Tissue Res. 2017; 369(3):445-454.
[10] Naegele M, Tillack K, Reinhardt S, Schippling S, Martin R, Sospedra M. Neutrophils in multiple sclerosis are characterized by a primed phenotype. J Neuroimmunol. 2012; 242(1-2):60-71.
[11] Rivers T M. Observations on attempts to produce acute disseminated encephalomyelitis in monkeys. J Exp Med. 1933; 58(1):39-53.
[12] Richard J F, Roy M, Audoy-Rémus J, Tremblay P, Vallières L. Crawling phagocytes recruited in the brain vasculature after pertussis toxin exposure through IL6, ICAM1 and ITGαM. Brain Pathol. 2011; 21(6): 661-671.
[13] Carlson T, Kroenke M, Rao P, Lane T E, Segal B. The Th17-ELR+ CXC chemokine pathway is essential for the development of central nervous system autoimmune disease. J Exp Med. 2008; 205(4): 811-823.
[14] Wu F, Cao W, Yang Y, Liu A. Extensive infiltration of neutrophils in the acute phase of experimental autoimmune encephalomyelitis in C57BL/6 mice. Histochem Cell Biol. 2010; 133(3):313-322.
[15] Aubé B, Lévesque S A, Paré A, et al. Neutrophils Mediate Blood-Spinal Cord Barrier Disruption in Demyelinating Neuroinflammatory Diseases. J Immunol. 2014; 193(5):2438-2454.
[16] Pun P B L, Lu J, Moochhala S. Involvement of ROS in BBB dysfunction. Free Radic Res. 2009; 43(4): 348-364.
[17] Zhang H, Ray A, Miller N M, Hartwig D, Pritchard K A, Dittel B N. Inhibition of myeloperoxidase at the peak of experimental autoimmune encephalomyelitis restores blood-brain barrier integrity and ameliorates disease severity. J Neurochem. 2016; 136(4):826-836.
[18] Karin Steinbach, Melanie Piedavent, Simone Bauer, Johannes T. Neumann, and Manuel A. Friese. Neutrophils Amplify Autoimmune Central Nervous System Infiltrates by Maturing Local APCs. J Immunol 2013; 191:4531-4539.
[19] Mallel E, Furtmuller P G, Sattler W, Obinger C. Myeloperoxidase: a target for new drug development. British Journal of Pharmacology 2007; 152, 838-854.
[20] WO2015148716.
[21] Van Der Veen B S, Myeloperoxidase: molecular mechanisms of action and their relevance to human health and disease. Antioxidants & redox signaling, 2009, 11.11: 2899-2937.
[22] YU, Guoliang; SHIKAN ZHENG, Hao Zhang. Inhibition of myeloperoxidase by N-acetyl lysyltyrosylcysteine amide reduces experimental autoimmune encephalomyelitis-induced injury and promotes oligodendrocyte regeneration and neurogenesis in a murine model of progressive multiple sclerosis. Neuroreport, 2018, 29.3: 208.
   Acknowledgement of the above references herein is not to be inferred as meaning that these are in any way relevant to the patentability of the presently disclosed subject matter.

### BACKGROUND OF THE INVENTION

Neutrophils are phagocytic granule-containing cells spanning the bloodstream, and are known as central mediators of the innate immune response. Upon early inflammatory initiation signals, resulting from bacterial infection or tissue damage, neutrophils migrate from the bloodstream, through the endothelial cell barrier and towards the site of damage and mediate early inflammatory response. This response is primarily attributed to phagocytosis of bacterial cells or foreign substances as well as to the release of cytokines and granules that contain proteolytic enzymes and reactive oxygen species (ROS) [1]. A more recently established feature of neutrophils is their ability to form neutrophil extracellular traps (NETs). The production of NETs (known as NETosis) is a tightly controlled mechanism by which following chromatin decondesatation, DNA is released from the cell, together with multiple bactericidal proteins to form net-like traps [2].

Myeloperoxidase (MPO) is a heme-containing peroxidase expressed by several myeloid cells, and is the most common protein expressed by neutrophils (comprises 5% of a neutrophils net weight). MPO catalyzes the formation of the toxic substance hypochlorous acid (HClO) as well as other oxidative substances [3]. Recently, MPO titration on murine neutrophils was shown to promote chromatin de-condensation and NET formation. Still further, it was shown that neutrophils derived from human donors with MPO deficiency were unable to produce NETs, indicating that MPO is essential for NET formation [4].

MPO was shown to represent a key factor in a number of conditions, for example cardiovascular diseases, inflammatory diseases, neurodegenerative diseases, kidney diseases and immune-mediated diseases [5-6]. Van Der Been et al. reports the implication of MPO in several diseases, especially those characterized by acute or chronic inflammation [21].

More specifically, Alzheimer's Disease (AD) and dementia are progressive neurodegenerative diseases characterized by global cognitive decline involving memory, orientation, judgment, and reasoning and is the most common form of dementia in the elderly Immune cells have long been known to be involved in AD pathogenesis and reactive glial cell were previously described by Alois Alzheimer. Astrocytes and microglial cells were extensively studied in the context of AD and are known to cluster around amyloid-β plaques. When these cells become activated, they secrete a variety of pro-inflammatory mediators, potentially causing oxidative damage to neuronal and surrounding vascular tissue and promote chronic inflammation. Although once regarded as less relevant to brain physiology and pathology, the role of peripheral immune cells in AD has been under growing investigation in recent years. This role was strongly supported by genome-wide association studies performed in the last years identifying multiple loci within immune-related genes expressed by myeloid cells as susceptibility loci for AD. Recently, Zenaro and colleagues reported that brain-penetrated neutrophils promote AD-like pathology in two mice models of AD by releasing the pro-inflammatory cytokine IL-17, and by producing NETs. Neutrophil depletion or inhibition of neutrophil migration into the CNS resulted in improved cognition and memory, as well as reduced neuroinflammation and Aβ burden. Finally, they also reported that NET-forming neutrophils are elevated in the brains of AD patients as opposed to healthy individuals [7]. Moreover, NETs release from neutrophils was also shown to be triggered by amyloid fibrils, supporting the formation of NETs in the neurodegenerative brain [8], and MPO immune-reactive cells were reported in the brains of AD and PD patients, in specific regions of neurodegeneration [9]. Moreover, brain-penetrated neutrophils were shown to promote AD-like pathology in two mice models of AD associated with releasing the pro-inflammatory cytokine IL-17, and by producing NETs. On the other hand, neutrophil depletion using anti-LY6G antibody or inhibition of neutrophil migration into the CNS resulted in improved cognition and memory, as well as reduced neuroinflammation and Amyloid-β levels. More recently, enhanced neutrophil adhesion in cortical capillaries was reported in the brain of AD model mice, resulting in reduced cerebral blood flow.

Still further, Multiple sclerosis (MS), the most common neurodegenerative disease of young adults, is a chronic auto-immune disease characterized by immune attack of the myelin sheath in the brain and spinal cord, mostly mediated by T-cell infiltration through the brain-blood barrier (BBB). While the etiology of MS, unlike non-autoimmune neurodegenerative diseases, comprises both innate and acquired immune-cells, several evidence indicates the involvement of neutrophil-mediated tissue damage. Neutrophils derived from MS patients exhibit a primed phenotype along with increased degranulation and NET formation and enhanced oxidative burst [10]. Multiple evidence also indicate the involvement of neutrophils in the experimental autoimmune encephalomyelitis (EAE) mouse model of MS [11]. During EAE, neutrophils are recruited to brain vasculature [12], invade the CNS [13], and accumulate within areas of demyelination and axonal damage [14]. Moreover, neutrophils were implicated in disruption of the BBB and the brain-spinal cord barrier (BSCB), and the administration of anti-neutrophil antibody was shown to impede EAE development [15].

Endothelial barrier disruption by neutrophils may allow intrusion of leukocytes into the CNS and promote EAE/MS lesions. Neutrophils were shown to accumulate in the CNS shortly before the onset of clinical symptoms and to correlate with lesion localization [13]. Endothelial barrier disruption by neutrophils was suggested to occur by the release of reactive oxygen species (ROS) [16]. Accordingly, inhibition of MPO during EAE was shown to restore BBB integrity and improve disease severity [17]. However, preclinical single-dose Antibody-mediated depletion of neutrophils delayed the onset and continuous depletion attenuated the development of experimental autoimmune encephalomyelitis, whereas the generation of a myelin-specific T cell response remained unaffected. In this study it was further found that neutrophil-related enzymes such as myeloperoxidase and neutrophil elastase did not contribute in mounting CNS inflammation, as analyzed by using respective knockout mice and inhibitors [18-19].

Several strategies were previously used to inhibit the activity MPO. For example azide, hydrazides and hydroxamic acids were used to inhibit MPO by modifying the heme site [7]. Still further, Yu et al. describes a specific inhibitor of MPO, N-acetyl lysyltyrosylcysteine amide (KYC) for the treatment of Multiple sclerosis using the EAE mouse mode [22]. WO2015148716 discloses methods and compositions for ex vivo expansion of hematopoietic cells using combinations of small molecule drugs and cytokines/growth factors. Multiple genes were influenced, among them MPO [20].

As being directed at modifying the heme active site of MPO, the prior art inhibitors fail to address undesired functions mediated by other domains of MPO. Moreover, the use of such inhibitors cannot prevent the formation of anti-MPO antibodies that may lead to vasculitis in pathologies such as AD. There is therefore a need to provide safe, efficient and specific methods and compositions to specifically and effectively target MPO, and various activities thereof in subjects suffering from MPO-related conditions.

### SUMMARY OF THE INVENTION

In accordance with a first aspect, the present disclosure provides methods of modulating the expression and/or activity of Myeloperoxidase (MPO) in a mammalian subject. More specifically, the method of the invention may comprise the step of administering to the subject an effective amount of at least one of: (a) at least one gene editing compound that is capable of or is adapted for modulating the expression and/or activity of MPO in at least one undifferentiated bone marrow (BM) cell of the subject. Alternatively or additionally, the method may administer to the subject (b), undifferentiated BM cell population exhibiting a modulated expression and/or activity of MPO. It should be noted that the administration of (a) and (b) or any composition or kit that comprise at least one of (a) and (b), is also encompassed by the methods of the invention.

In accordance with further aspects, the present disclosure provides methods for treating, preventing, ameliorating, inhibiting or delaying the onset of an MPO-related condition in a mammalian subject. The methods of the invention may comprise the step of administering to the treated subject a therapeutically effective amount of at least one of: (a) at least one gene editing compound capable of modulating the expression and/or activity of MPO in at least one undifferentiated BM cell of the subject; and (b), undifferentiated BM cell population exhibiting a modulated expression and/or activity of MPO. The administration of (a) and (b) or any composition or kit that comprise at least one of (a) and (b), is also encompassed by the methods of the invention.

In some further aspects thereof, the invention provides kits comprising at least one of: (a) at least one gene editing compound that is capable of, or is adapted for, modulating the expression and/or activity of MPO in at least one undifferentiated BM cell of the subject; and (b), undifferentiated BM cell population exhibiting a modulated expression and/or activity of MPO. In accordance with some further aspects, the present disclosure provides a pharmaceutical composition comprising a therapeutic effective amount of at least one of: (a) at least one gene editing compound that is capable of, or is adapted for, modulating the expression and/or activity of MPO in at least one undifferentiated BM cell of the subject; and (b), undifferentiated BM cell population exhibiting a modulated expression and/or activity of MPO. The composition may optionally further comprise at least one of pharmaceutically acceptable carrier/s, diluent/s and/or excipient/s.

Still further aspect of the invention relates to at least one undifferentiated BM cell transduced or transfected with at least one gene editing compound capable of modulating the expression and/or activity of MPO in said cell.

Other aspects of the invention relate to at least one of the compositions, the kits and the cells of the invention for use in methods for modulating the expression and/or activity of MPO. Still further aspects of the invention provide at least one of the compositions, the kits and the cells of the invention for use in therapeutic methods for treating, preventing, ameliorating, inhibiting or delaying the onset of an MPO-related condition in a mammalian subject.

In yet some further aspect, the invention provides therapeutically effective amount of at least one of: (a) at least one gene editing compound adapted for modulating the expression and/or activity of MPO in at least one undifferentiated BM cell of said subject; and (b) undifferentiated BM cell population exhibiting a modulated expression and/or activity of MPO, for use in a method for treating, preventing, ameliorating, inhibiting or delaying the onset of an MPO-related condition in a mammalian subject. The invention further provides methods for inhibiting NETosis and related conditions. These and other aspects of the invention will become apparent by the hand of the following description.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1: Establishment of the Experimental Autoimmune Encephalomyelitis (EAE) model in C57BL/6 mice Clinical scores of C57BL/6 female mice induced to an EAE disease at the indicated Myelin oligodendrocyte glycoprotein 35-55 (MOG35-55) and pertussis toxin (PT) concentrations. N≥4 mice per group.

FIG. 2: Bone marrow (BM) transplantation rescue lethality in irradiated C57BL/6 mice Weight follow up of 2 month old male C57/BL6 mice subjected to lethal radiation (9 Gy) followed by transplantation of BM cells from WT mice, Lin-enriched BM cells, or saline transplantation.

FIGS. 3A-3F: Transplantation of BM from MPO^{Tm1/Lus} into 5×FAD mice

FIG. 3A: Graph showing Weight follow up of 2 months old non-transgenic littermates subjected to irradiation and transplantation of BM derived from WT mice.

FIG. 3B: Graph showing Weight follow up of 2 months old non-transgenic littermates subjected to irradiation and transplantation of BM derived from Mpo^{Tm1/Lus} mice.

FIG. 3C: Graph showing Weight follow up of 2 months old 5×FAD transgenic mice subjected to irradiation and transplantation of BM derived from WT mice.

FIG. 3D: Graph showing Weight follow up of 2 months old 5×FAD transgenic mice subjected to irradiation and transplantation of BM derived from Mpo^{Tm1/Lus} mice.

FIG. 3E: Peroxidase activity assay of blood in WT BM-transplanted mice.

FIG. 3F: Peroxidase activity assay of blood in Mpo^{Tm1/Lus}-BM transplanted mice

FIG. 4A-4C: Generation of 5×FAD mice with bone marrow deficient with MPO FIG. 4A: Schematic representation of mice groups: 8-week-old 5×FAD mice and their non-transgenic littermates were lethally irradiated and transplanted with bone marrow derived from either WT C57BL/6 or MPO^{tm1/lus} mice. Numbers of mice in each group are indicated in brackets.

FIG. 4B: MPO mRNA expression analysis of bone marrow cells derived from 10-month-old mice. Data are mean±SEM. *P<0.05, **P<0.01, ***P<0.001, ****P<0.0001. Two-tailed t-test.

FIG. 4C: Schematic timeline of the Experimental design.

FIG. 5A-5E: MPO deficiency inhibit increase in anxiety-related behavior in 5×FAD mice

FIG. 5A: Graph showing time spent in corners of seven-month old mice subjected to open field maze test.

FIG. 5B: Graph showing time spent in the center of seven-month old mice subjected to open field maze test.

FIG. 5C: Graph showing the total distance traveled by the mice.

FIG. 5D: Graph showing time spent in the open arms of mice subjected to the elevated plus maze test.

FIG. 5E: Graph showing time spent in the closed arms of mice subjected to the elevated plus maze test.

Data are mean±SEM. *P<0.05, **P<0.01, ***P<0.001, ****P<0.0001. Two-tailed t-test.

FIG. 6A-6E: MPO deficiency protects against learning and memory decline in 5×FAD mice

FIG. 6A: Graph showing novel-arm exploration relative frequency of 7-month old mice using a two-trial Y-maze test (indicative of short-term spatial memory).

FIG. 6B: Graph showing novel-arm exploration relative time of 7-month old mice using a two-trial Y-maze test (indicative of short-term spatial memory).

FIG. 6C: Graph showing escape latency of 8-month old mice in the Morris water maze test (indicative of spatial learning).

FIG. 6D: Graph showing the total distance covered of 8-month old mice in the Morris water maze test (indicative of spatial learning).

FIG. 6E: Graph showing freezing behavior in the fear conditioning test of 9-month old mice. Data are mean±SEM. *P<0.05, **P<0.01, ***P<0.001, ****P<0.0001. Two-tailed t-test.

FIG. 7A-7E: MPO deficiency does not affect amyloid-β plaque load in 5×FAD mice

FIG. 7A: Representative image of hippocampal slices stained with DAPI/ThioS of WT mice transplanted with WT bone marrow cells. Scale bar=200 μm.

FIG. 7B: Representative image of hippocampal slices stained with DAPI/ThioS of 5×FAD mice transplanted with WT bone marrow cells. Scale bar=200 μm.

FIG. 7C: Representative image of hippocampal slices stained with DAPI/ThioS of WT mice transplanted with MPO-KO bone marrow cells. Scale bar=200 μm.

FIG. 7D: Representative image of hippocampal slices stained with DAPI/ThioS of 5×FAD mice transplanted with MPO-KO bone marrow cells. Scale bar=200 μm.

FIG. 7E: Graph showing quantification of ThioS plaque density. Data are mean±SEM. *P<0.05, **P<0.01, ***P<0.001, ****P<0.0001. Two-tailed Mann-Whitney test.

FIG. 8A-8N: Low S100β expression in 5×FAD MPO KO mice

FIG. 8A: Representative image of hippocampal slices stained with DAPI of WT mice transplanted with WT bone marrow cells.

FIG. 8B: Representative image of hippocampal slices stained with DAPI of 5×FAD mice transplanted with WT bone marrow cells.

FIG. 8C: Representative image of hippocampal slices stained with DAPI of WT mice transplanted with MPO-KO bone marrow cells.

FIG. 8D: Representative image of hippocampal slices stained with DAPI of 5×FAD mice transplanted with MPO-KO bone marrow cells.

FIG. 8E: Representative image of hippocampal slices stained with S100β of WT mice transplanted with WT bone marrow cells.

FIG. 8F: Representative image of hippocampal slices stained with S100β of 5×FAD mice transplanted with WT bone marrow cells.

FIG. 8G: Representative image of hippocampal slices stained with S100β of WT mice transplanted with MPO-KO bone marrow cells.

FIG. 8H: Representative image of hippocampal slices stained with S100β of 5×FAD mice transplanted with MPO-KO bone marrow cells.

FIG. 8I: Representative image of hippocampal slices stained with DAPI/S100β of WT mice transplanted with WT bone marrow cells.

FIG. 8J: Representative image of hippocampal slices stained with DAPI/S100β of 5×FAD mice transplanted with WT bone marrow cells.

FIG. 8K: Representative image of hippocampal slices stained with DAPI/S100β of WT mice transplanted with MPO-KO bone marrow cells.

FIG. 8L: Representative image of hippocampal slices stained with DAPI/S100β of 5×FAD mice transplanted with MPO-KO bone marrow cells.

FIG. 8M: Graph showing quantification of S100β mean intensity.

FIG. 8N: Graph showing S100β mRNA expression analysis of Hippocampal samples. Data are mean±SEM. *P<0.05, **P<0.01, ***P<0.001, ****P<0.0001. Two-tailed Mann-Whitney test.

FIG. 9A-9E: Low levels of inflammatory markers in 5×FAD MPO KO mice

FIG. 9A: Graph showing mRNA expression analysis of IL1β in hippocampal samples.

FIG. 9B: Graph showing mRNA expression analysis of CXCL10 in hippocampal samples.

FIG. 9C: Graph showing mRNA expression analysis of CCL2 in hippocampal samples.

FIG. 9D: Graph showing mRNA expression analysis of TNFα in hippocampal samples.

FIG. 9E: Graph showing mRNA expression analysis of VCAM1 in hippocampal samples.

Data are mean±SEM. *P<0.05, **P<0.01, ***P<0.001, ****P<0.0001. Two-tailed Mann-Whitney test.

FIG. 10A-10C: Low APOE levels in 5×FAD MPO KO mice

FIG. 10A: Picture of western blot analysis of hippocampal cell lysates probed with anti APOE antibody.

FIG. 10B: Graph showing western blot analysis data represented as % of each sample to the WT-WT group average.

FIG. 10C: Graph showing mRNA expression analysis of APOE in hippocampal samples.

Data are mean±SEM. *P<0.05, **P<0.01, ***P<0.001, ****P<0.0001. Two-tailed Mann-Whitney test.

FIG. 11A-11N: Plasmid cloning and lentiviral preparation

FIG. 11A: FACS analysis of isolated Lin⁻ cells, non-transduced, following 24 hours.

FIG. 11B: FACS analysis of isolated Lin⁻ cells, transduced with 10 μL of GFP encoding particles following 24 hours.

FIG. 11C: FACS analysis of isolated Lin⁻ cells, transduced with 10 μL of CRISPR/Ca9 encoding lentiviral particles targeting gRNA2 (SEQ ID NO:5) in the MPO gene, following 24 hours.

FIG. 11D: FACS analysis of isolated Lin⁻ cells, transduced with 10 μL of CRISPR/Ca9 encoding lentiviral particles targeting gRNA6 (SEQ ID NO:6) in the MPO gene, following 24 hours.

FIG. 11E: FACS analysis of isolated Lin⁻ cells, non-transduced, following 48 hours.

FIG. 11F: FACS analysis of isolated Lin⁻ cells, transduced with 2 μl of GFP encoding particles following 48 hours.

FIG. 11G: FACS analysis of isolated Lin⁻ cells, transduced with 2 μl of CRISPR/Ca9 encoding lentiviral particles targeting gRNA2 (SEQ ID NO:5) in the MPO gene, following 48 hours.

FIG. 11H: FACS analysis of isolated Lin⁻ cells, transduced with 2 μl of CRISPR/Ca9 encoding lentiviral particles targeting gRNA6 (SEQ ID NO:6) in the MPO gene, following 48 hours.

FIG. 11I: FACS analysis of isolated Lin⁻ cells, transduced with 5 μl of GFP encoding particles following 48 hours.

FIG. 11J: FACS analysis of isolated Lin⁻ cells, transduced with 5 μl of CRISPR/Ca9 encoding lentiviral particles targeting gRNA2 (SEQ ID NO:5) in the MPO gene, following 48 hours.

FIG. 11K: FACS analysis of isolated Lin⁻ cells, transduced with 5 μl of CRISPR/Ca9 encoding lentiviral particles targeting gRNA6 (SEQ ID NO:6) in the MPO gene, following 48 hours.

FIG. 11L: FACS analysis of isolated Lin⁻ cells, transduced with 10 μl of GFP encoding particles following 48 hours.

FIG. 11M: FACS analysis of isolated Lin⁻ cells, transduced with 10 μl of CRISPR/Ca9 encoding lentiviral particles targeting gRNA2 (SEQ ID NO:5) in the MPO gene, following 48 hours.

FIG. 11N: FACS analysis of isolated Lin⁻ cells, transduced with 10 μl of CRISPR/Ca9 encoding lentiviral particles targeting gRNA6 (SEQ ID NO:6) in the MPO gene, following 48 hours.

FIG. 12: T7 Analysis of MPO cleavage

Picture of agarose gel showing the products of a T7 analysis of full length DNA and cleavage products. Gel shows untreated and sorted or unsorted cells edited with Cas9 and crRNA: tracrRNA, for each of the crRNA used.

Lane A: Untreated 293 cells, amplified with primers surrounding H502 loci.

Lane B: 293 cells treated with RNP anti T173.

Lane C: 293 cells treated with RNP anti D260.

Lane D: 293 cells treated with RNP anti H502.

Lane E: 293 cells treated with RNP anti T173, enriched for fluorescently labeled tracrRNA.

Lane F: 293 cells treated with RNP anti D260, enriched for fluorescently labeled tracrRNA.

Lane G: 293 cells treated with RNP anti H502, enriched for fluorescently labeled tracrRNA.

Lane H: T7 negative control.

Lane I: T7 positive control.

### DETAILED DESCRIPTION OF THE INVENTION

Myeloperoxidase (MPO) is a heme-containing protein involved in the body defense mechanism against infections and is primarily located in the granules of neutrophils. MPO is involved in a variety of immune-mediated disease, neurodegenerative disease and inflammatory disease.

The MPO RNA is detected only at the bone marrow and it is only the MPO protein that reaches various target tissues in the body. This transcription of the MPO gene, occurring in the bone marrow, is turned on early during the myoblast stage of bone marrow myeloid cell differentiation and is turned off at the time myeloid precursors are induced to differentiate.

It was suggested by the inventors that by specifically affecting the gene expression of MPO in primitive immature cells of the bone marrow, it is possible to modulate MPO expression and subsequent MPO activity eventually resulting in neutrophils having modulated MPO (eliminate/inhibit or alternatively, establish/increase MPO expression and/or activity). Specific bone marrow on-site gene modulation of MPO in immature cells, is therefore suggested by the inventors as an effective, selective and accurate method for affecting MPO expression and subsequent activity in the neutrophils and/or macrophages or any other cells associated with the immune-system or components thereof. Interestingly, while some pathologies are associated with accumulation of MPO in various organs in the body such as the brain, the modulation of MPO as described herein is performed by gene editing of MPO encoding gene or transcript in undifferentiated BM, cells and not in the target organ (for example the brain or any components thereof). Thus, the method described herein of MPO protein modulation in neutrophils and/or macrophages via gene editing either in vivo or ex vivo, in the BM cells provides a broad method of modulation that is not limited to a specific target tissue. In other words, by specifically modulating (e.g., depleting) MPO in BM cells, thereby modulating the immune-system of the subject, the invention provides powerful method for treating disorders that affect other organs or tissues not necessarily connected directly to the immune-system.

It is further suggested that such methods described herein are advantageous over other methods, for example using MPO protein inhibitor, such as azide, hydrazides and hydroxamic acids for various reasons. Firstly, substantive modulation of MPO activity using known MPO protein inhibitors requires constant administration in order to address the on-going formation of neutrophils and/or macrophages that express MPO. Secondly, known MPO protein inhibitors are typically designed to bind to the MPO active site (e.g., the hemoglobin binding site) thereby affecting the protein activity, specifically, the peroxidase activity. As such, this approach is limited to MPO functions that are mediated by its active site. In this manner, modulation of other MPO activities, for example inhibition of NETosis as further detailed below, or any other activities of MPO disclosed by the invention, specifically, activities that are not mediated by the MPO active site, is not addressed. In other words, MPO activities mediated by other MPO sites, other than the binding site cannot be modulated using known MPO inhibitors. Moreover, the use of such inhibitors cannot prevent the formation of anti-MPO antibodies that may lead to vasculitis in pathologies such as AD.

As such, the present disclosure is based on the novel concept of modulating the expression and/or activity of MPO in undifferentiated BM cells for modulating MPO in a subject in need, and further, the development of compounds that modulate MPO gene expression at the transcription site, namely the bone marrow and specifically in the primitive, immature undifferentiated cells of the bone marrow. Thus, by targeted modulation of the immune system of a subject by specifically modulating (e.g., eliminating) the levels, activity or expression of MPO, the invention provides methods affecting MPO levels and/or activity in other organs or tissues (e.g., brain), and uses thereof for effective treatment of disorders associated directly or indirectly with MPO expression and/or activity (enhanced, reduced or even at the normal level). More specifically, by manipulating the immune system of the treated subject in vivo or replacing said immune system with cells (e.g., BM cells, that may differentiate to form neutrophils and/or macrophages) that exhibit modulated MPO expression, the invention provide tools for affecting MPO expression and/or activity in other organs and tissues of the treated subject, thereby treating disorders or conditions that may be associated or affected by MPO expression and/or activity in different organs or tissues.

As demonstrated by the present invention, bone marrow cells having modulated MPO expression can serve as an efficient platform in allogenic or autologous bone marrow transplantation.

In addition, the compounds developed herein as well as the modulated bone marrow cell population can be used for treating a variety of conditions associated directly or indirectly with MPO expression and/or activity. For example, disorders or conditions associated with low MPO expression and/or activity, conditions associated with high MPO expression and/or activity, or even conditions that may exhibit normal levels, expression and/or activity of MPO, but may be affected either by elevating or by reducing MPO levels, expression or activities.

Thus, in accordance with a first aspect, the present disclosure provides a method of modulating the expression and/or activity of Myeloperoxidase (MPO) in a mammalian subject. More specifically, the methods of the invention may comprise the step of administering to the subject an effective amount of at least one of: (a) at least one gene editing compound capable of and/or is adapted for modulating the expression and/or activity of MPO in at least one undifferentiated bone marrow (BM) cell of the subject; and (b) undifferentiated BM cell population exhibiting a modulated expression and/or activity of MPO. It should be further appreciated that the invention further provides an effective amount of at least one of: (a) at least one gene editing compound capable of and/or is adapted for modulating the expression and/or activity of MPO in at least one undifferentiated bone marrow (BM) cell of the subject; and (b) undifferentiated BM cell population exhibiting a modulated expression and/or activity of MPO, for use a method of modulating the expression and/or activity of Myeloperoxidase (MPO) in a mammalian subject. The invention provides methods, compositions, cells, kits and systems for modulating the expression and/or activity of MP1. Myeloperoxidase (MPO), as used herein, is the most toxic enzyme found in the azurophilic granules of neutrophils. MPO utilizes H₂O₂ to generate hypochlorous acid (HClO) and other reactive moieties, which kill pathogens during infections. The MPO gene is located on the long arm segment q12-24 of chromosome 17 and the primary transcriptional product of this gene consists of 11 introns and 12 exons. Alternative splicing of the MPO mRNA gives two transcripts of 3.6 and 2.9 kB. The primary translation product is an 80 kDa precursor protein that undergoes a series of modifications including cleavage of a signal peptide, N-linked glycosylation, and limited deglycosylation, to form the catalytically inactive MPO precursor (apoproMPO). In the next step, MPO gains catalytic activity by incorporation of an iron-heme molecule into the catalytic centrum. Heme is covalently attached by two ester bonds and, unique for heme containing enzymes, a third sulfonium linkage, that uniquely orients one heme molecule into the enzyme pocket. The unique configuration of the heme moiety confers MPO with very high oxidative potential, enabling chlorination at physiological pH. Cleavage of proMPO leads to a 59 kDa α-subunit and a 13.5 kDa β-subunit that are covalently attached through the heme moiety. A disulfide bridge joins the two heavy-light protomers in mature 150 kDa MPO. MPO expression levels depend upon allelic polymorphisms in the promoter region. A G to A substitution at position −463 (G-463A) leads to a 25-fold decrease in MPO transcription. The substitution occurs within the Alu receptor response element (AluRRE), which is a cluster of nuclear receptor biding sites. The substitution changes the consensus sequence of the Sp1 transcription factor, which is essential for enhanced MPO transcription. A G to A substitution, leading to decreased MPO transcription is also found at position −129 (G-129A). Neutrophils are the main source of MPO where it accounts for 5% of the dry weight of the cell, making MPO the most abundant protein in neutrophils. MPO is transcribed only in promyelocytes during neutrophil differentiation in the bone marrow. MPO expression is induced by G-CSF, which promotes differentiation of multipotential progenitor cells to granulocytes by regulating the transcription factors C-EBP and PU.1, which drive commitment of the granulocyte-macrophage progenitor to neutrophils and macrophages. Neutrophil differentiation leads to decreased MPO transcription, which is undetectable in mature cells.

In promyelocytes, MPO is packaged into azurophilic granules along with several other antimicrobial proteins, serine proteases and lysosome hydrolases. The packaging of proteins in the azurophilic granules is asynchronous creating heterogeneous populations of azurophilic granules. In promyelocytes there are at least three categories of azurophilic granules. Small electron dense and nucleated azurophilic granules have uniform MPO distribution, while MPO in large spherical granules is detected at the granule rim.

In addition, MPO is synthesized by promyelomonocytes, where it accounts for 1% of total cell protein. Monocytes released into the circulation turn off MPO synthesis, which is further downregulated during their differentiation into macrophages.

It should be further appreciated that in the context of the present disclosure, unless specifically indicated “MPO” encompasses both the MPO gene and the MPO protein. The human MPO gene has a sequence as provided by Accession Number: NC_000017.11. In yet some further specific embodiment, such sequence may comprise the nucleic acid sequence as denoted by SEQ ID NO: 1. In yet some further embodiments, the human MPO protein is denoted by Accession Number: NP_000241. Still further in some embodiments, the MPO protein may comprise the amino acid sequence as denoted by SEQ ID NO: 2. Still further in some other specific embodiment, the invention further provides the mouse MPO encoding sequence as denoted by Accession Number NC_000077, that may in some embodiments comprise the nucleic acid sequence as denoted by SEQ ID NO: 15. In yet some further embodiments, the mouse MPO protein is denoted by Accession Number: NP_034954. Still further in some embodiments, the mouse MPO protein may comprise the amino acid sequence as denoted by SEQ ID NO: 16.

The methods of the invention are based on modulation of MPO levels and/or activity in a subject in need by the provision of a gene editing system that modulate the expression and/or activity of MPO in undifferentiated BM cells of a subject (either in vivo or ex vivo). Alternatively, such modulation may be performed by providing BM cells having a modulated MPO expression and/or activity (specifically obtained from an allogeneic subject).

As used herein the term undifferentiated bone marrow cell or immature bone marrow cell refers to a cell in the bone marrow that has the ability to transform into specialized cell types with specific characteristics. The bone marrow (BM) is a soft, spongy, gelatinous tissue found in the hollow spaces in the interior of bones that forms an important role in the lymphatic system and specifically in the development of the cells of the blood system.

In some embodiments, the undifferentiated BM cell may be a hematopoietic stem cell (HSC). HSCs are primitive immature undifferentiated cells in the BM, which are capable of self-renewal and differentiation. The HSCs divide into the myeloid and lymphoid lineages of blood cells via the myeloid progenitor and the lymphoid progenitor, respectively to produce all the mature blood cells including the three classes of blood cells that are found in circulation: white blood cells (leukocytes), red blood cells (erythrocytes), and platelets (thrombocytes). In some embodiments, the undifferentiated BM cell is a progenitor cell. Progenitor cells are the main functional component of the bone marrow and include the progenitor cells, which are destined to mature into blood, and lymphoid cells. In some embodiments, the undifferentiated BM cell is a common myeloid progenitor (CMP). CMP also denoted as myeloid stem cells are the first branch of cell differentiation after the HSC. Myeloid and lymphoid lineages both are involved in dendritic cell formation. Myeloid cells include monocytes, macrophages, neutrophils, basophils, eosinophils, erythrocytes, and megakaryocytes to platelets. Lymphoid cells include T cells, B cells, and (natural killer cells).

HSCs and progenitor cells can be identified or isolated for example by the use of flow cytometry by using combination of several different cell surface markers. For example, the HSCs lack expression of mature blood cell markers. In addition, both HSCs and progenitor cells can be identified and isolated, for example, by specific molecules denoted as cluster of differentiation (CD).

More specifically, cluster of differentiation as used herein, refers to a nomenclature protocol used for the identification and investigation of cell surface molecules providing targets for immuno-phenotyping of cells. CD molecules act as cell receptors or ligands important to the cell.

In some embodiments, the undifferentiated BM cells may be characterized by being CD34 positive cells (CD34⁺). Cluster of differentiation 34 (CD34) is a transmembrane phosphoglycoprotein protein present on hematopoietic stem cells as a cell surface glycoprotein and is associated with selection and enrichment of hematopoietic stem cells for bone marrow transplants. In addition, CD34 functions as a cell-cell adhesion factor and may also mediate the attachment of hematopoietic stem cells to bone marrow extracellular matrix or directly to stromal cells. In yet some other embodiments, the undifferentiated BM cells may be characterized by being CD38 positive cells (CD38⁺). Cluster of differentiation 38 (CD38) also known as cyclic ADP ribose hydrolase is a glycoprotein found on the surface of many immune cell and has been used as a prognostic marker in leukemia.

In some embodiments, the undifferentiated BM cell may be a HSC or any progenitor cell. In some embodiments, the undifferentiated BM cell may comprise any combination of HSC and progenitor cells. In some embodiments, the undifferentiated BM cell may comprise any combination of HSC and CMP. Still further, in some embodiments, the methods and compositions of the invention may modulate the MPO expression, levels and/or activity in CD34⁺ that may maturate into neutrophiles. In yet some further embodiments, the methods and compositions of the invention may modulate the MPO expression, levels and/or activity in CD34⁺ that may maturate into macrophages. Thus, it should be understood that in some embodiments the methods and compositions of the invention offer modulation of the activity and function of macrophages and/or neutrophiles.

In accordance with the present disclosure, the mammalian subject may be administered either with a gene editing system capable of modulating MPO expression and/or activity in BM cells, thereby offering an in vivo modulation of MPO, or alternatively, or additionally, with cells that display modified expression or activity of MPO, thereby providing an ex vivo modulation. It should be noted that the invention further encompasses MPO modulation in a subject that may comprise in vivo and ex vivo approaches and any combinations thereof. Such administration results in the modulation of the expression and/or activity of MPO in the subject. The gene-editing compound as used herein refers a compound that is capable of modulating at least a single gene in the body. The term “modulating” encompasses any change or modification in at least one gene in the body such as an increase or decrease of expression and/or activity in relation to a control or a normal or a baseline level of expression/activity determined under certain condition. In some embodiments, the gene editing compounds or system, or alternatively, the undifferentiated BM cell population administered to the subject may lead to inhibition or elimination of the expression and/or activity of MPO in the subject.

It should be noted that in some alternative embodiments, the gene editing compounds/system, or alternatively, the undifferentiated BM cell population administered to the subject may lead to establishment, enhancement or increase the expression and/or activity of MPO in the subject. Thus, by targeted manipulation of the immune-system of the subject, a general manipulation of the activity and/or levels and expression of MPO in various organs and tissues of the subjects is achieved. Moreover, by specific and targeted manipulation of the immune-system of the subjects, disorders that are associated with MPO- are treated.

In more specific embodiments, at least one gene editing compound or system may be administered for modulating the activity and/or expression of MPO in the subject. The gene-editing compound can modulate the expression and/or activity of MPO by various mechanism. For example, the gene-editing compound modulate MPO expression and/or activity by genetic modification of the chromosomal DNA that results in knockdown cell, organism and the like. In addition, the gene editing compound/system may modulate MPO expression and/or activity by a transient/temporary modification (transient knockdown).

The gene editing compound or system used by the methods of the invention (as well as in the compositions, cells, kits and uses described herein after), may be in some embodiments, a natural occurring compound (such as an enzyme, short DNA or RNA oligonucleotide), a synthetic compound or an artificial compound. In some embodiments, the at least one gene editing compound may be an oligonucleotide. Such oligonucleotide can bind to an active gene or any transcripts thereof and cause decreased expression by, for example blocking of transcription (in the case of gene-binding by anti-sense oligonucleotides), the degradation of the mRNA transcript (e.g. by small interfering RNA (siRNA)) or RNase-H dependent antisense), or through the blocking of either mRNA translation, pre-mRNA splicing sites, or nuclease cleavage sites used for maturation of other functional RNAs, including miRNA (e.g. by morpholino oligos or other RNase-H independent antisense oligonucleotide).

In some further embodiments, the gene editing compound of the invention may comprise any nucleic acid modifier protein and at least one target recognition element. More specifically, in yet some further embodiments, the gene editing system applicable in the invention may comprise at least one nucleic acids-modifier protein and at least one target recognition element. Such recognition element may comprise in some embodiments nucleic acid sequence that specifically targets the modifier protein to the target gene, specifically, gene encoding the MPO in accordance with the invention. In some embodiments, nucleic acids-modifier protein may include any protein or protein complex that modifies an encoding or non-encoding nucleic acid sequence. In yet some further embodiments, the modification caused by said modifier protein may modulate the expression of the MPO protein product encoded or alternatively, controlled by the target nucleic acid sequence of such modifier. Alternatively, the modifier applicable in the invention may modulate the stability or the activity of such MPO. Such modification include nucleolytic cleavage (e.g., by a nuclease), methylation, demethylation, (of either coding or non-coding sequences, such as control elements) activation or repression of protein expression and the like. Thus, in some embodiments, the nucleic acid modifier may be at least one of a nuclease, methyl transferase, demethylase, transcription factor, transcription repressor, any fusion proteins thereof, and any complex comprising at least one of said modifier and any combinations thereof. In some embodiments, the modifier protein of the invention may be at least one nuclease. It should be appreciated that the term “nuclease” as used herein relates in some embodiments to an active nuclease having a nucleolytic activity. However, it should be appreciated that in some embodiments, the term “nuclease” as used herein further encompasses a molecule having the structural features of a nuclease but display reduced, defective or no nucleolytic activity on any nucleic acid molecule, specifically, DNA or RNA (referred to herein as an inactive nuclease). The inactive-nuclease as used herein further encompasses any fragment, mutant or fusion protein of an inactive nuclease. More specifically, a fusion protein of an inactive nuclease with any other protein e.g., transcription factor or repressor, methyl transferase, demethylase, as will be elaborated herein after.

More specifically, as used herein, the term “nuclease” is an enzyme that in some embodiments display a nucleolytic activity, specifically, capable of cleaving the phosphodiester bonds between monomers of nucleic acids (e.g., DNA and/or RNA). Nucleases variously effect single and double stranded breaks in their target molecules. There are two primary classifications based on the locus of activity. Exonucleases digest nucleic acids from the ends. Endonucleases act on regions in the middle of target molecules. They are further subcategorized as deoxyribonucleases and ribonucleases. The former acts on DNA, the latter on RNA. A nuclease must associate with a nucleic acid before it can cleave the molecule, providing a degree of recognition. The nucleases belong just like phosphodiesterase, lipase and phosphatase to the esterases, a subgroup of the hydrolases. This subgroup includes the Exonucleases are enzymes that work by cleaving nucleotides one at a time from the end (exo) of a polynucleotide chain. A hydrolyzing reaction that breaks phosphodiester bonds at either the 3′ or the 5′ end occurs. Eukaryotes and prokaryotes have three types of exonucleases involved in the normal turnover of mRNA: 5′ to 3′ exonuclease (Xrn1), which is a dependent decapping protein; 3′ to 5′ exonuclease, an independent protein; and poly(A)-specific 3′ to 5′ exonuclease. Members of this family include Exodeoxyribonucleases producing 5′-phosphomonoesters, Exoribonucleases producing 5′-phosphomonoesters, Exoribonucleases producing 3′-phosphomonoesters and Exonucleases active with either ribo- or deoxy-. Members of this family include exonuclease, II, III, IV, V, VI, VII, and VIII. As noted above, Endonucleases are enzymes that cleave the phosphodiester bond within a polynucleotide chain. Some endonucleases, such as deoxyribonuclease I, cut DNA relatively nonspecifically (without regard to sequence), while many, typically called restriction endonucleases or restriction enzymes, cleave only at very specific nucleotide sequences.

In some embodiment, the nuclease may be an active enzyme having a nucleolytic activity as specified above. In some alternative embodiments, the nuclease may be a defective enzyme.

A defective enzyme (e.g., a defective mutant, variant or fragment) may relate to an enzyme that display an activity reduced in about 1%, 2%, 3%, 4%, 5% to about 100%, specifically, about 5% to about 10%, about 10% to about 15%, about 15% to about 20%, about 20% to about 25%, about 25% to about 30%, about 35% to about 40%, about 40% to about 45%, about 45% to about 50%, about 50% to about 55%, about 55% to about 60%, about 65% to about 70%, about 75% to about 80%, about 80% to about 85%, about 85% to about 90%, about 90% to about 95%, about 95% to about 99.9%, more specifically, reduced activity of about 98% to about 100% as compared to the active nuclease.

As indicated above, the gene editing system of the invention further comprises at least one target recognition element. As used herein a “target recognition element” is a nucleic acid sequence (either RNA or DNA) that will direct the nucleic acid-modifier protein, for example, the nuclease to a specific target position within a nucleic acid sequence. The recognition of the target by the target recognition element is facilitated in some embodiments by base-pairing interactions. These target recognition elements are specifically relevant in case of guided nucleases In some embodiments, for nucleases displaying a nucleolytic activity, directing the nuclease to a specific site may result in cleaving the phosphodiester bonds between monomers of nucleic acids (e.g., DNA and/or RNA) that may lead in some embodiments to specific destruction thereof. In yet some alternative embodiments, where a non-active nuclease is used, and specifically, a fusion protein thereof, directing such defective nuclease by a target recognition element, may result in targeted modulation (e.g., activation or repression, methylation or demethylation) of the target nucleic acid sequence that is targeted by the target recognition element. It should be noted that a target recognition element may comprise between about 10 nucleotides to 70 nucleotides or more. In yet some further embodiments, the target recognition element may comprise a number of nucleotides as specified for the spacers herein after. In certain embodiments, the nuclease used by the methods of the invention may be a guided nuclease. In yet some other embodiments, the gene editing compound may comprise a polypeptide, specifically, an enzyme. In some embodiments, the gene-editing compound may comprise at least one programmable-engineered nuclease (PEN) or any variants or proteins thereof. The term “programmable engineered nucleases (PEN)” as used herein also known as “molecular DNA scissors”, refers to enzymes either synthetic or natural, used to replace, eliminate or modify sequences in a highly targeted way. PEN target and cut specific genomic sequences (recognition sequences) such as DNA sequences. The at least one PEN may be derived from natural occurring nucleases or may be an artificial enzyme, all involved in DNA repair of double strand DNA lesions and enabling direct genome editing. In some alternative or additional embodiments the gene editing compound according with the present disclosure encompasses also any nucleic acid molecule comprising at least one nucleic acid sequence encoding the PEN or any kit, composition or vehicle comprising the at least one PEN, or any nucleic acid sequence encoding PEN. In some embodiments, the at least one PEN may be at least one of a mega nuclease, a zinc finger nuclease (ZFN), a transcription activator-like effector-based nuclease (TALEN), a Fok1 nuclease, or a clustered regularly interspaced short palindromic repeats (CRISPR/Cas) system, or any fusion proteins or chimeras thereof.

In some embodiments, the at least one PEN may be a mega nuclease. Mega nucleases are endodeoxyribonucleases characterized by a large recognition site (double-stranded DNA sequences of 12 to 40 base pairs); such that this site generally occurs only once in any given genome. Meganucleases are specific naturally occurring restriction enzymes and include among others, the LAGLIDADG family of homing endonucleases, mostly found in the mitochondria and chloroplasts of eukaryotic unicellular organisms. In some embodiments, the at least one PEN may be a megaTAL. MegaTALs are fusion proteins that combine homing endonucleases, such as LAGLIDADG family, with the modular DNA binding domains of TALENs.

In some embodiments, the nuclease used by the methods and compositions of the invention may be a guided nuclease. In yet some specific embodiments, the nuclease is at least one Transcription activator-like effector nucleases (TALEN). TALEN are restriction enzymes that can be engineered to cut specific sequences of DNA. They are made by fusing a TAL effector DNA-binding domain to a DNA cleavage domain of a nuclease. More specifically, TALENs are artificial endonucleases designed by fusing the DNA-binding domain (multiples of nearly identical repeats each comprised of about 34 amino acids) obtained from TAL (transcription activator-like) effector (TALE) protein to the cleavage domain of the Fok1 endonuclease. Each TALE repeat independently recognizes its corresponding nucleotide (nt) base with two variable residues [termed the repeat variable di-residues (RVDs)] such that the repeats linearly represent the nucleotide sequence of the binding site.

In yet some further alternative embodiments, the guided nuclease that is used by the methods and compositions of the invention may be at least one Zinc-finger nucleases (ZFNs). ZFNs are artificial restriction enzymes generated by fusing a zinc finger DNA-binding domain to a DNA-cleavage domain Zinc finger domains can be engineered to target specific desired DNA sequences and this enables zinc-finger nucleases to target unique sequences within complex genomes. More specifically, the ZFNs are artificial endonucleases that have been generated by combining a small zinc finger (ZF; about 30 amino acids) DNA-binding/recognition domain (Cys₂His₂) to a type IIS nonspecific DNA-cleavage domain from the Fok1 restriction enzyme. However, the cleavage activity of the Fok1 endonuclease demands dimerization. As a ZF module recognizes a 3 bp sequence, there is a requirement for multiple fingers in each ZFN monomer for recognizing and binding to longer DNA target sequences.

In yet some other embodiments, the guided nuclease used by the methods and compositions of the invention may be Fok1 nuclease or any fusion proteins thereof as will be discussed herein after. The enzyme Fok1 (Fok-1), naturally found in Flavobacterium okeanokoites, is a bacterial type IIS restriction endonuclease consisting of an N-terminal DNA-binding domain and a non-specific DNA cleavage domain at the C-terminal. In accordance with some embodiments of the invention, Fok1 may be directed to its target (e.g., at the MPO gene) by a target recognition element. Such target recognition element as discussed herein above, may be in some embodiments, a nucleic acid sequence that specifically directs Fok1 to the target site. The recognition element may be connected directly or indirectly (i.e., via a linker), to the Fok1. Thus, once the protein is bound to duplex DNA via the target recognition element, the DNA cleavage domain is activated and cleaves, the first strand 9 nucleotides downstream and the second strand 13 nucleotides upstream of the nearest nucleotide of the target site. More specifically, in case Fok1 is used as the nucleic acid modifier protein of the invention, specific targeting to MPO encoding sequences may be achieved by directly or indirectly (e.g., via a linker) binding to the Fok1, at least one target recognition element, that may be at least one nucleic acids sequence (e.g., RNA or DNA), that specifically hybridizes to the target site. Still further, in some embodiments, the at least one PEN may be a clustered regularly interspaced short palindromic repeats (CRISPR/Cas) system. In some embodiments, the methods described herein uses the CRISPR system as a PEN for modulating MPO expression and/or activity. The Clustered Regularly Interspaced Short Palindromic Repeats (CRISPR) system is a bacterial immune system that has been modified for genome engineering.

In accordance with some specific embodiments, the methods described herein may use PEN that may comprise at least one clustered regulatory interspaced short palindromic repeat (CRISPR)/CRISPR associated (cas) protein system. Thus, in more specific embodiments the methods of the invention may comprise the step of administering to said subject an effective amount of at least one of: (a) at least one CRISPR/cas protein, or any nucleic acid molecule encoding said Cas protein; and (b), at least one nucleic acid sequence comprising at least one guide RNA (gRNA) that targets a protospacer within the MPO gene, or any nucleic acid sequence encoding said gRNA. The method of the invention may optionally use any kit, composition or vehicle comprising at least one of (a) and (b).

It should be noted that when both elements of the CRISPR/Cas system of the invention are provided as nucleic acid sequences, they may be provided either separately in two or more nucleic acid molecules or alternatively, together in a single nucleic acid molecule that comprises both sequences, specifically, construct, vector or any vehicle comprising both, (a) and (b).

As indicated above, the invention may provide in some embodiments thereof, the elements of the gene-editing system (e.g., the gRNA and/or the Cas protein), or at least parts thereof, as nucleic acid sequences or molecules. The term “nucleic acid”, “nucleic acid sequence”, or “polynucleotide” and “nucleic acid molecule” refers to polymers of nucleotides, and includes but is not limited to deoxyribonucleic acid (DNA), ribonucleic acid (RNA), DNA/RNA hybrids including polynucleotide chains of regularly and/or irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides, wherein the attachment of various entities or moieties to the nucleotide units at any position are included. The terms should also be understood to include, as equivalents, analogs of either RNA or DNA made from nucleotide analogs, and, as applicable to the embodiment being described, single-stranded (such as sense or antisense) and double-stranded polynucleotides. Preparation of nucleic acids is well known in the art. It should be noted that the nucleic acid molecules (or polynucleotides) according to the invention can be produced synthetically, or by recombinant DNA technology. Methods for producing nucleic acid molecules are well known in the art. The nucleic acid molecule according to the invention may be of a variable nucleotide length. For example, in some embodiments, the nucleic acid molecule according to the invention comprises 1-100 nucleotides, e.g., about 10, 20, 30, 40, 50, 60, 70, 80, 90 or 100 nucleotides. In other embodiments the nucleic acid molecule according to the invention comprises 100-1,000 nucleotides, e.g., about 100, 200, 300, 400, 500, 600, 700, 800, 900 or 1000 nucleotides. In further embodiments the nucleic acid molecule according to the invention comprises 1,000-10,000 nucleotides, e.g., about 1,000, 2,000, 3,000, 4,000, 5,000, 6,000, 7,000, 8,000, 9,000 or 10,000 nucleotides. In yet further embodiments the nucleic acid molecule according to the invention comprises more than 10,000 nucleotides, for example, 20,000, 30,000, 40,000, 50,000, 60,000, 70,000, 80,000, 90,000 or 100,000 nucleotides.

As noted above, the nucleic acid molecules may be provided by the invention comprised within constructs, vectors or any other vehicle. Vectors, as used herein, are nucleic acid molecules of particular sequence that can be introduced into a host cell, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Many vectors, e.g. plasmids, cosmids, minicircles, phage, viruses, (as detailed below) useful for transferring nucleic acids into target cells, specifically the BM cells, may be applicable in the present invention. The vectors comprising the nucleic acid(s) may be maintained episomally, e.g. as plasmids, minicircle DNAs, transposons, viruses such cytomegalovirus, adenovirus, or they may be integrated into the target cell genome, through homologous recombination or random integration, e.g. retrovirus-derived vectors such as lentiviruses, AAV, MMLV, HIV-1, ALV, etc. More specifically, in some embodiments, the vector may be a viral vector. In yet some particular embodiments, such viral vector may be any one of recombinant adeno associated vectors (rAAV), single stranded AAV (ssAAV), self-complementary rAAV (scAAV), Simian vacuolating virus 40 (SV40) vector, Adenovirus vector, helper-dependent Adenoviral vector, retroviral vector and lentiviral vector.

More specifically, as indicated above, in some embodiments, viral vectors may be applicable in the present invention. The term “viral vector” refers to a replication competent or replication-deficient viral particle which are capable of transferring nucleic acid molecules into a host.

The term “virus” refers to any of the obligate intracellular parasites having no protein-synthesizing or energy-generating mechanism. The viral genome may be RNA or DNA contained with a coated structure of protein of a lipid membrane. Examples of viruses useful in the practice of the present invention include baculoviridiae, parvoviridiae, picornoviridiae, herepesviridiae, poxviridiae, adenoviridiae, picotmaviridiae. The term recombinant virus includes chimeric (or even multimeric) viruses, i.e. vectors constructed using complementary coding sequences from more than one viral subtype.

In yet some specific embodiments, lentiviral vectors may be used in the present invention. Lentiviral vectors are derived from lentiviruses which are a subclass of Retroviruses. Commonly used retroviral vectors are “defective”, i.e. unable to produce viral proteins required for productive infection. Rather, replication of the vector requires growth in a packaging cell line. To generate viral particles comprising the nucleic acids sequence of the invention, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing the retroviral vectors comprising the nucleic acids sequence of the invention into packaging cell lines and of collecting the viral particles that are generated by the packaging lines are well known in the art.

Still further in some embodiments, a viral vector applicable in the present invention may be the Adeno-associated virus (AAV). The term “adenovirus” is synonymous with the term “adenoviral vector”. AAV is a single-stranded DNA virus with a small (˜20 nm) protein capsule that belongs to the family of parvoviridae, and specifically refers to viruses of the genus adenoviridiae. The term adenoviridiae refers collectively to animal adenoviruses of the genus mastadenovirus including but not limited to human, bovine, ovine, equine, canine, porcine, murine and simian adenovirus subgenera. In particular, human adenoviruses includes the A-F subgenera as well as the individual serotypes thereof the individual serotypes and A-F subgenera including but not limited to human adenovirus types 1, 2, 3, 4, 4a, 5, 6, 7, 8, 9, 10, 11 (AdllA and Ad IIP), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91.

Due to its inability to replicate in the absence of helpervirus coinfections (typically Adenovirus or Herpesvirus infections) AAV is often referred to as dependovirus. AAV infections produce only mild immune responses and are considered to be nonpathogenic, a fact that is also reflected by lowered biosafety level requirements for the work with recombinant AAVs (rAAV) compared to other popular viral vector systems. Due to its low immunogenicity and the absence of cytotoxic responses AAV-based expression systems offer the possibility to express genes of interest for months in quiescent cells.

Production systems for rAAV vectors typically consist of a DNA-based vector containing the DNA of interest, which is flanked by inverted terminal repeats. Construct sizes are limited to approximately 4.7-5.0 kb, which corresponds to the length of the wild-type AAV genome. rAAVs are produced in cell lines. The expression vector is co-transfected with a helper plasmid that mediates expression of the AAV rep genes which are important for virus replication and cap genes that encode the proteins forming the capsid. Recombinant adeno-associated viral vectors can transduce dividing and non-dividing cells, and different rAAV serotypes may transduce diverse cell types. These single-stranded DNA viral vectors have high transduction rates and have a unique property of stimulating endogenous Homologous Recombination without causing double strand DNA breaks in the host genome.

The invention in some embodiments thereof utilizes the CRISPR system for specific modulation of MPO gene. As indicated above, the gene editing system of the invention may be provided as nucleic acid molecules, specifically in a delivery vector or vehicle. However, it should be appreciated that any of the gene editing systems used, may be also administered as a protein complex, or alternatively, as a ribonucleoprotein complex as will be discussed herein after in connection with specific gene editing systems applicable in the present case.

As indicated above, in some embodiments, the gene editing system applicable in the present invention is the CRISPR-Cas system. CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and class 2 may be divided into types II, V, and VI.

Thus, in some embodiments, the Cas protein may be a member of at least one of CRISPR-associated system of Class 1 and Class 2. In some embodiments, the cas protein may be a member of at least one of CRISPR-associated system of any one of type II, type I, type III, type IV, type V and type VI. As used herein, CRISPR arrays also known as SPIDRs (Spacer Interspersed Direct Repeats) constitute a family of DNA loci that are usually specific to a particular bacterial species. The CRISPR array is a distinct class of interspersed short sequence repeats (SSRs) that were first recognized in E. coli. In subsequent years, similar CRISPR arrays were found in Mycobacterium tuberculosis, Haloferax mediterranei, Methanocaldococcus jannaschii, Thermotoga maritima and other bacteria and archaea. It should be understood that the invention contemplates the use of any of the known CRISPR systems, particularly any of the CRISPR systems disclosed herein. The CRISPR-Cas system has evolved in prokaryotes to protect against phage attack and undesired plasmid replication by targeting foreign DNA or RNA. The CRISPR-Cas system, targets DNA molecules based on short homologous DNA sequences, called spacers that exist between repeats. These spacers guide CRISPR-associated (Cas) proteins to matching (and/or complementary) sequences within the foreign DNA, called proto-spacers, which are subsequently cleaved. The spacers can be rationally designed to target any DNA sequence, for example the MPO gene sequence. Moreover, this recognition element may be designed separately to recognize and target any desired target.

In some specific embodiment, the RNA guided DNA binding protein nuclease of the system of the invention may be a CRISPR Class 2 system. In yet some further particular embodiments, such class 2 system may be a CRISPR type II system.

The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cast and Cast. Cas9 contains at least two nuclease domains, a RuvC-like nuclease domain near the amino terminus and the HNH (or McrA-like) nuclease domain in the middle of the protein, but the function of these domains remains to be elucidated. However, as the HNH nuclease domain is abundant in restriction enzymes and possesses endonuclease activity responsible for target cleavage.

Still further, it should be noted that type II system comprise at least one of cas9, cas1, cas2 csn2, and cas4 genes. It should be appreciated that any type II CRISPR-Cas systems may be applicable in the present invention, specifically, any one of type II-A or B. Thus, in yet some further and alternative embodiments, at least one cas gene used in the methods, compositions, cells, kits, uses and systems of the invention may be at least one cas gene of type II CRISPR system (either typeII-A or typeII-B). In more particular embodiments, at least one cas gene of type II CRISPR system used by the methods and systems of the invention may be the cas9 gene. It should be appreciated that such system may further comprise at least one of cas1, cas2, csn2 and cas4 genes. Thus, in some specific embodiments, the Cas protein use by the methods of the invention may be Cas9 or any fragments, mutants, variants or derivatives thereof.

Double-stranded DNA (dsDNA) cleavage by Cas9 is a hallmark of “type II CRISPR-Cas” immune systems. The CRISPR-associated protein Cas9 is an RNA-guided DNA endonuclease that uses RNA:DNA complementarity to a target site (proto-spacer). After recognition between Cas9 and the target sequence double stranded DNA (dsDNA) cleavage occur, creating the double strand brakes (DSBs).

CRISPR type II system as used herein requires the inclusion of two essential components: a “guide” RNA (gRNA) and a non-specific CRISPR-associated endonuclease (Cas9). The gRNA is a short synthetic RNA composed of a “scaffold” sequence necessary for Cas9-binding (also named tracrRNA) and about 20 nucleotide long “spacer” or “targeting” sequence, which defines the genomic target to be modified. Guide RNA (gRNA), as used herein refers to a synthetic fusion of the endogenous tracrRNA with a targeting sequence (also named crRNA), providing both scaffolding/binding ability for Cas9 nuclease and targeting specificity. Also referred to as “single guide RNA” or “sgRNA”.

CRISPR was originally employed to “knock-out” target genes in various cell types and organisms, but modifications to the Cas9 enzyme have extended the application of CRISPR to “knock-in” target genes, selectively activate or repress target genes, purify specific regions of DNA, and even image DNA in live cells using fluorescence microscopy. Furthermore, the ease of generating gRNAs makes CRISPR one of the most scalable genome editing technologies and has been recently utilized for genome-wide screens. In most of CRISPR systems, the target sequence within the genome to be edited, should be present immediately upstream of a Protospacer Adjacent Motif (PAM). In other systems, such as type III, there is no PAM.

In CRISPR systems based on PAM sequence recognition like CRISPR Type II, the PAM is absolutely necessary for target binding and the exact sequence is dependent upon the species of Cas9 (5′ NGG 3′ for Streptococcus pyogenes Cas9). In some embodiments, the nucleic acid modifier protein used in the methods of the invention is Cas9. In certain embodiments, Cas9 from S. pyogenes may be used in the methods and systems of the invention. Nevertheless, it should be appreciated that any known Cas9 may be applicable. Non-limiting examples for Cas9 useful in the present disclosure include but are not limited to Streptococcus pyogenes (SP), also indicated herein as SpCas9, Staphylococcus aureus (SA), also indicated herein as SaCas9, Neisseria meningitidis (NM), also indicated herein as NmCas9, Streptococcus thermophilus (ST), also indicated herein as StCas9 and Treponema denticola (TD), also indicated herein as TdCas9. Still further, in some embodiments of the present disclosure, the endonuclease may be a Cas9, Cas13, Cas6, Cpf1, CMS1 protein, or any variant thereof that is derived or expressed from Methanococcus maripaludis C7, Corynebacterium diphtheria, Corynebacterium efficiens YS-314, Corynebacterium glutamicum (ATCC 13032), Corynebacterium glutamicum (ATCC 13032), Corynebacterium glutamicum R, Corynebacterium kroppenstedtii (DSM 44385), Mycobacterium abscessus (ATCC 19977), Nocardia farcinica IFM10152, Rhodococcus erythropolis PR4, Rhodococcus jostii RFIA1, Rhodococcus opacus B4 (uid36573), Acidothermus cellulolyticus 11 B, Arthrobacter chlorophenolicus A6, Kribbella flavida (DSM 17836), Thermomonospora curvata (DSM43183), Bifidobacterium dentium Bd1, Bifidobacterium longum DJO10A, Slackia heliotrinireducens (DSM 20476), Persephonella marina EX H 1, Bacteroides fragilis NCTC 9434, Capnocytophaga ochracea (DSM 7271), Flavobacterium psychrophilum JIP02 86, Akkermansia muciniphila (ATCC BAA 835), Roseiflexus castenholzii (DSM 13941), Roseiflexus RS1, Synechocystis PCC6803, Elusimicrobium minutum Pei191, uncultured Termite group 1 bacterium phylotype Rs D17, Fibrobacter succinogenes S85, Bacillus cereus (ATCC 10987), Listeria innocua, Lactobacillus casei, Lactobacillus rhamnosus GG, Lactobacillus salivarius UCC118, Streptococcus agalactiae-5-A909, Streptococcus agalactiae NEM316, Streptococcus agalactiae 2603, Streptococcus dysgalactiae equisimilis GGS 124, Streptococcus equi zooepidemicus MGCS10565, Streptococcus gallolyticus UCN34 (uid46061), Streptococcus gordonii Challis subst CH1, Streptococcus mutans NN2025 (uid46353), Streptococcus mutans, Streptococcus pyogenes M1 GAS, Streptococcus pyogenes MGAS5005, Streptococcus pyogenes MGAS2096, Streptococcus pyogenes MGAS9429, Streptococcus pyogenes MGAS 10270, Streptococcus pyogenes MGAS6180, Streptococcus pyogenes MGAS315, Streptococcus pyogenes SSI-1, Streptococcus pyogenes MGAS10750, Streptococcus pyogenes NZ131, Streptococcus thermophiles CNRZ1066, Streptococcus thermophiles LMD-9, Streptococcus thermophiles LMG 18311, Clostridium botulinum A3 Loch Maree, Clostridium botulinum B Eklund 17B, Clostridium botulinum Ba4 657, Clostridium botulinum F Langeland, Clostridium cellulolyticum H10, Finegoldia magna (ATCC 29328), Eubacterium rectale (ATCC 33656), Mycoplasma gallisepticum, Mycoplasma mobile 163K, Mycoplasma penetrans, Mycoplasma synoviae 53, Streptobacillus, moniliformis (DSM 12112), Bradyrhizobium BTAil, Nitrobacter hamburgensis X14, Rhodopseudomonas palustris BisB18, Rhodopseudomonas palustris BisB5, Parvibaculum lavamentivorans DS-1, Dinoroseobacter shibae. DFL 12, Gluconacetobacter diazotrophicus Pal 5 FAPERJ, Gluconacetobacter diazotrophicus Pal 5 JGI, Azospirillum B510 (uid46085), Rhodospirillum rubrum (ATCC 11170), Diaphorobacter TPSY (uid29975), Verminephrobacter eiseniae EF01-2, Neisseria meningitides 053442, Neisseria meningitides alpha14, Neisseria meningitides Z2491, Desulfovibrio salexigens DSM 2638, Campylobacter jejuni doylei 269 97, Campylobacter jejuni 81116, Campylobacter jejuni, Campylobacter lari RM2100, Helicobacter hepaticus, Wolinella succinogenes, Tolumonas auensis DSM 9187, Pseudoalteromonas atlantica T6c, Shewanella pealeana (ATCC 700345), Legionella pneumophila Paris, Actinobacillus succinogenes 130Z, Pasteurella multocida, Francisella tularensis novicida U 112, Francisella tularensis holarctica, Francisella tularensis FSC 198, Francisella tularensis tularensis, Francisella tularensis WY96-3418, or Treponema denticola (ATCC 35405).

In some specific and non-limiting embodiments, the Cas9 of Streptococcus pyogenes M1 GAS, specifically, the Cas9 of protein id: AAK33936.1, may be applicable in the methods, compositions, cells, kits, uses and systems of the invention. In some embodiments, the Cas9 protein may be encoded by the nucleic acid sequence as denoted by SEQ ID NO: 3. In further specific embodiments, the Cas9 protein may comprise the amino acid sequence as denoted by SEQ ID NO: 4, or any derivatives, mutants, variants or any fusion proteins thereof. Once expressed, the Cas9 protein and the gRNA provided by the methods and compositions of the invention, form a riboprotein complex through interactions between the gRNA “scaffold” domain and surface-exposed positively-charged grooves on Cas9. Cas9 undergoes a conformational change upon gRNA binding that shifts the molecule from an inactive, non-DNA binding conformation, into an active DNA-binding conformation. Importantly, the “spacer” sequence of the gRNA remains free to interact with target DNA. The Cas9-gRNA complex binds any target genomic sequence with a PAM, but the extent to which the gRNA spacer matches the target DNA determines whether Cas9 will cut, or alternatively, perform any other manipulation in case a fusion protein comprising a catalytically inactive cas9 is used. In yet some further specific embodiments, Base editor enzymes may be use by the compositions and methods of the invention. More specifically, CAS9-Based base editors are enzymes, specifically synthetic and non-naturally occurring enzymes, that utilize catalytically inactive CAS9 fused to an enzyme catalyzing a specific nucleotide substitution. Known base editors are able to substitute C to T, A to G, T to C, and G to A. The ‘base editing’ enzymes enable the direct, irreversible conversion of one target DNA base into another in a programmable manner, without requiring dsDNA backbone cleavage or a donor template. More particularly, these chimeric enzymes (also referred to herein as fusion proteins), are based on fusion of CRISPR/Cas9 and a cytidine deaminase enzyme that retain the ability to be targeted by a guide RNA, however, cannot induce dsDNA breaks. These enzymes therefore mediate the direct conversion of cytidine to uridine, thereby effecting a C→T (or G→A) substitution. Still further, once the Cas9-gRNA complex binds a putative DNA target, a “seed” sequence at the 3′ end of the gRNA targeting sequence begins to anneal to the target DNA. If the seed and target DNA sequences match, the gRNA continues to anneal to the target DNA in a 3′ to 5′ direction.

Cas9 will only cleave the target if sufficient homology exists between the gRNA spacer and target sequences. Still further, the Cas9 nuclease has two functional endonuclease domains: RuvC and HNH. Cas9 undergoes a second conformational change upon target binding that positions the nuclease domains to cleave opposite strands of the target DNA. The end result of Cas9-mediated DNA cleavage is a double strand break (DSB) within the target DNA that occurs about 3 to 4 nucleotides upstream of the PAM sequence.

The resulting DSB may be then repaired by one of two general repair pathways, the efficient but error-prone Non-Homologous End Joining (NHEJ) pathway and the less efficient but high-fidelity Homology Directed Repair (HDR) pathway. Programmable engineered nucleases (PEN) strategies for genome editing, may be based either on cell activation of the HDR mechanism following specific double stranded DNA cleavage (knock-in system) or on NHEJ mechanism (knock-out system).

In some specific embodiments, the targeted genes to be knockout, specifically, the MPO gene, are repaired through the NHEJ pathway, resulting in most cases in dysfunction of the target genes (deletions/insertions/non-sense mutations etc.). As discussed previously, Cas9 generates double strand breaks (DSBs) through the combined activity of two nuclease domains, RuvC and HNH. The exact amino acid residues within each nuclease domain that are critical for endonuclease activity are known (D10A for HNH and H840A for RuvC in S. pyogenes Cas9) and modified versions of the Cas9 enzyme containing only one active catalytic domain (called “Cas9 nickase”) have been generated. Cas9 nickases still bind DNA based on gRNA specificity, but nickases are only capable of cutting one of the DNA strands, resulting in a “nick”, or single strand break, instead of a DSB. DNA nicks are rapidly repaired by HDR (homology directed repair) using the intact complementary DNA strand as the template. Thus, two nickases targeting opposite strands are required to generate a DSB within the target DNA (often referred to as a “double nick” or “dual nickase” CRISPR system). This requirement dramatically increases target specificity, since it is unlikely that two off-target nicks will be generated within close enough proximity to cause a DSB. It should be therefore understood, that the invention further encompasses the use of any mutants, variants or derivatives of Cas9, including the dual nickase approach to create a double nick-induced DSB for increasing specificity and reducing off-target effects. It should be therefore understood, that the invention further encompasses the use of the dual nickase approach to create a double nick-induced DSB for increasing specificity and reducing off-target effects, in the methods, cells and compositions of the invention. Specific examples of increasing specificity is the use of a nuclease such as Fok1 fused to dCas9 that serves as a linker to the targeting gRNA. More specifically, the well-characterized, dimerization-dependent Fok1 nuclease domain is fused to a RNA-guided catalytically inactive Cas9 (dCas9) protein, thereby providing enhanced specificity due to the dimerization required for the nuclease activity of the Fok1. In yet another embodiment, the invention further encompasses the option of providing a pre-crRNA that can be processed to several final gRNA products that may target identical or different targets, specifically, the MPO gene. In yet some more specific embodiments, the crRNA comprised within the gRNA of the invention may be a single-stranded ribonucleic acid (ssRNA) sequence complementary to a target genomic DNA sequence. In some specific embodiments, the target genomic DNA sequence within the MPO gene may be located immediately upstream of a protospacer adjacent motif (PAM) sequence and further.

In some embodiments, the gRNAs or crRNAs used by the methods, compositions, cells, kits, uses and systems of the invention may target a protospacer comprising the nucleic acid sequence as denoted by any one of SEQ ID NOs. 43, 44, 45, 46, 94, 95, 96, 97, 98, 99, 100, 101, 102, 103, 104, 105 and 106 of the human MPO gene, or any fragments thereof. In yet some further embodiments, the gRNA used by the methods, compositions, cells, kits, uses and systems of the invention may target a protospacer comprising the nucleic acid sequence as denoted by any one of SEQ ID NOs. 5, 6, 8, 9, 10, 11, 12, 13, 14, of the mouse MPO gene, or any fragments thereof. Still further, in some embodiments, the gRNA used by the methods of the invention may target a protospacer comprising a nucleic acid sequence corresponding to various sites in the MPO gene that encode various domains in the MPO protein, specifically, the human MPO gene and protein. In some particular embodiments, targeting specific site in MPO gene may result in either knocking out the entire MPO molecule or alternatively, modulate its function and activity, specifically when HDR is used for replacement of specific residues or sequences within the MPO gene. In some embodiments, the gRNA used by the methods of the invention may target a protospacer comprising a nucleic acid sequence corresponding to the active site of MPO. In more specific embodiments, the gRNAs of the invention, specifically when directed to modulate the human MPO protein, levels, expression and/or activity, may target protospacers located at different domains of MPO. In some specific embodiments, of the human MPO that comprises the amino acid sequence as denoted by SEQ ID NO. 2. Such domains or sequences may include domains or sequences required for protomer biosynthesis and processing, domains or specific residues that mimic human MPO deficiency, domains or sequences required for protomer heme binding and domains or sequences that required for proper protein glycosylation. In more specific embodiments, the gRNA used by the methods of the invention may target a protospacer encompassed by a domain or a protospacer comprising nucleic acid sequence encoding residues that are required for protomer biosynthesis and processing. Such sequences may encode domains that include residues or sequences residing between residues 120 to 190 or/and residues 320-380 of MPO, specifically of the human MPO amino acid sequence as denoted by SEQ ID NO. 2. A gRNA targeting any protospacer comprised within such domain or sequence encoding such residues, may therefore lead to conformational changes in the protein or changes in protomer biosynthesis and processing. Still further, in some embodiments, the gRNA used by the methods of the invention may target a protospacer encompassed by a domain or a protospacer comprising nucleic acid sequence encoding residues that mimic human MPO deficiency substitutions. Such protospacer may be located within a nucleic acid sequence encoding residues 150 to 260 of the human MPO, as denoted by SEQ ID NO. 2 and/or residues 480 to 580 of the human MPO, as denoted by SEQ ID NO. 2. In yet some further embodiments, the gRNA used by the methods of the invention may target a protospacer encompassed by a domain or a protospacer comprising nucleic acid sequence encoding residues that are required for protomer heme binding. In some specific embodiments, such protospaces may be located within a nucleic acid sequence encoding residues 250 to 270, and/or residues 390 to 510 of human MPO as denoted by SEQ ID NO. 2. In yet some further embodiments, the gRNA used by the methods of the invention may target a protospacer encompassed by a domain or a protospacer comprising nucleic acid sequence encoding residues required for proper protein glycosylation. Such protospacer may be located within a nucleic acid sequence encoding residues 300 to 360 of the human MPO as denoted by SEQ ID NO. 2. In some specific embodiments, the gRNAs or crRNAs used by the invention target specific protospaces in the human MPO gene. Additional target site within the human MPO gene that may be targeted by the methods, compositions and kits of the invention, may include residues C167, C180, C319, C158, R128, N355, T173, M251, R569, R499, G501, Q257, D260, M409, E408, H261, H502 and N355 of the human MPO, as denoted by SEQ ID NO. 2, as also indicated by Example 4. It should be noted that the present invention encompasses the use of each one of these residues as a target as discussed herein. In some further specific embodiments, the crRNA (gRNA) used as the target recognition element by the present invention may comprise the nucleic acid sequence as denoted by any one of SEQ ID NO. 33 (referred to herein as T173), SEQ ID NO:34 (referred to herein as D260), SEQ ID NO. 35 (referred to herein as H502), and SEQ ID NO. 42 (referred to herein as C319), that target the corresponding target protospaces within the human MPO. In some embodiments, the crRNA sequence of SEQ ID NO. 33, of the invention may target a sequence comprising the sequence TGCACATCCCGGTGATGGTG (also denoted by SEQ ID NO. 43). In some further embodiments, the crRNA sequence of SEQ ID NO. 34 of the invention may target a sequence comprising the sequence CAGGGGTGAAGTCGAGGTCG (also denoted by SEQ ID NO. 44). In yet some further embodiments, the crRNA sequence of SEQ ID NO. 35 of the invention may target a sequence comprising the sequence GGATGAGGGTGTGGCCGTAG (also denoted by SEQ ID NO. 45). In yet some further embodiments, the crRNA sequence of SEQ ID NO. 42 of the invention may target a sequence comprising the sequence GGATGGTGATGTTGCTCCCGGGG (also denoted by SEQ ID NO. 46). In yet some further embodiments, the crRNA (gRNA) used as the target recognition element by the present invention may comprise the nucleic acid sequence as denoted by any one of 47, 50, 55, 60, 63, 66, 71, 74, 77, 80, 85, 88 and 91. In yet some further embodiments, these gRNAs target the protospacers within the human MPO nucleic acid sequence. In more specific embodiments, these protospacers comprise the nucleic acid sequence as denoted by SEQ ID NOs. 94, 95, 96, 97, 98, 99, 100, 101, 102, 103, 104, 105, and 106, respectively.

In yet some other specific embodiments, the gRNAs or crRNAs used by the invention, target specific protospaces in the mouse MPO gene. In some embodiments, the gRNA sequence of the invention may target a protospacer sequence comprising the sequence CCCCAACGATCAGCTGACCA (also denoted by SEQ ID NO. 5). In some further embodiments, the gRNA sequence of the invention may target a sequence comprising the sequence CAGCGGGGTGTACGGCAGCG (also denoted by SEQ ID NO. 6). In yet some further embodiments, the gRNA sequence of the invention may target a sequence comprising the sequence GCACTCATGTTCATGCAGTG (also denoted by SEQ ID NO. 8). In some further embodiments, the gRNA sequence of the invention may target a sequence comprising the sequence TGCGATACTTGTCATTCGGT (also denoted by SEQ ID NO. 9). In some further embodiments, the gRNA sequence of the invention may target a sequence comprising the sequence AGTAAAACAGGAGCTCCGTG (also denoted by SEQ ID NO. 10). In some further embodiments, the gRNA sequence of the invention may target a sequence comprising the sequence ACGCTTCCAAGACAATGGCA (also denoted by SEQ ID NO. 11). In some further embodiments, the gRNA sequence of the invention may target a sequence comprising the sequence ACGCCATCTTCATACTCTGC (also denoted by SEQ ID NO. 12). In some further embodiments, the gRNA sequence of the invention may target a sequence comprising the sequence ATCAAGCGGAGCCTCCAAAG, (also denoted by SEQ ID NO. 13). In yet some further embodiments, the gRNA sequence of the invention may target a sequence comprising the sequence AGAGTACCTGTAACATTGAA (also denoted by SEQ ID NO. 14). In some embodiments, the gRNA comprises the nucleic acid sequence as denoted by any one of SEQ ID NO. 5, SEQ ID NO:6, SEQ ID NO. 8 and SEQ ID NO. 9, SEQ ID NO. 10, SEQ ID NO. 11, SEQ ID NO. 12, SEQ ID NO. 13, SEQ ID NO. 14, that target protospacer sequences as defined above of the mouse MPO. In some further embodiments, the crRNA used as the target recognition element by the present invention may comprise the nucleic acid sequence as denoted by any one of SEQ ID NO. 33, SEQ ID NO:34, SEQ ID NO. 35, SEQ ID NO. 42, as well as SEQ ID NOs. 47, 50, 55, 60, 63, 66, 71, 74, 77, 80, 85, 88 and 91.

As indicated herein, the gRNA transcribed by the transgene of the invention may be complementary, at least in part, to the target genomic DNA, specifically any protospacer within the MPO gene. In certain embodiments, “Complementarity” refers to a relationship between two structures each following the lock-and-key principle. In nature complementarity is the base principle of DNA replication and transcription as it is a property shared between two DNA or RNA sequences, such that when they are aligned antiparallel to each other, the nucleotide bases at each position in the sequences will be complementary (e.g., A and T or U, C and G). In yet some further embodiments, the gRNA or crRNA used by the methods of the invention may comprise a nucleic acid sequence comprising at least part of the nucleic acid sequence of the target protospacer.

As indicated above in some particular embodiments, the genomic DNA sequence targeted by the gRNA of the system of the invention may be located immediately upstream to a PAM sequence. According to some embodiments, the methods, compositions, cells, kits, uses and systems of the invention may encompass the use of several gRNAs, specifically, more than one gRNA. Thus, in some embodiments, the polynucleotide encoding the gRNA of the invention may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more, specifically, 110, 120, 130, 140, 150, 160, 170, 180, 190, 200 or more spacers. That is to say that in some embodiments the gRNA or crRNA encoding sequence may encode 1 to 200 or more identical or different gRNAs that target at least two or more different positions on the MPO encoding sequence. It should be further understood that the spacers of the nucleic acid sequence encoding the gRNA of the invention may be either identical or different spacers. In more embodiments, these spacers may target either an identical or different target protospacers within the MPO gene. In yet some other embodiments, such spacer may target at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more target protospacers within the MPO gene. These target sequences may be derived from a single region or part of the MPO gene or alternatively, from several target regions or parts of the MPO gene or encoding sequence.

As used herein, the term “spacer” refers to either a non-repetitive or repetitive spacer sequence that is designed to target a specific sequence. In some specific embodiments, spacers may comprise between about 15 to about 50 nucleotides, specifically, about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides. More specifically, spacers may comprise between about 20-35 nucleotides.

Generally, the guide or targeting RNA encoded by the CRISPR system of the invention may comprise a CRISPR RNA (crRNA) and a trans activating RNA (tracrRNA). However, it should be noted that in some specific CRISPR system, the guide RNA does not include a tracrRNA, such as CPF1 based CRISPR-Cas systems and CRISPR type I-E. The sequence of the targeting RNA encoded by the CRISPR spacers is not particularly limited, other than by the requirement for it to be directed to (i.e., having a segment that is the same as or complementarity to) a target sequence within the MPO gene that is also referred to herein as a “proto-spacer”. Such proto-spacers comprise nucleic acid sequence having sufficient complementarity to a targeting RNA encoded by the CRISPR spacers comprised within the nucleic acid sequence encoding the gRNA of the methods, compositions, cells, kits, uses and systems provided by the invention. Thus, in some embodiments, the encoded gRNA or crRNA comprise sequence that is identical at least in part to the nucleic acid sequence of the target protospacer. In some other embodiment, the RNA guided DNA binding protein nuclease of the system of the invention may be any one of a clustered regularly interspaced short palindromic repeat (CRISPR) of a newly identified system. It should be appreciated that the invention provides the use of any fragment, derivative, mutant or variant of Cas9, specifically of Cas9 that comprises the amino acid sequence as denoted by SEQ ID NO. 4, or any fragments, variants, derivatives and mutants thereof, or any chimera or fusion proteins thereof. It should be appreciated that the methods, compositions, cells, kits and systems of the invention may provide in some embodiments thereof, as a gene-editing system, the Cas protein, specifically the Cas9 protein. It should be noted that “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, manosylation, amidation, carboxylation, sulfhydryl bond formation, cleavage and the like. By “fragments or peptides” it is meant a fraction of said Cas9 protein. A “fragment” of a molecule, such as any of the amino acid sequences of the present invention, is meant to refer to any amino acid subset of the Cas9 protein. This may also include “variants” or “derivatives” thereof. A “peptide” is meant to refer to a particular amino acid subset having functional activity. By “functional” is meant having the same biological function, for example, having the ability to perform a specific RNA-guided nuclease reaction.

It should be appreciated that the invention encompasses any variant or derivative of the Cas9 proteins of the invention and any polypeptides that are substantially identical or homologue. The term “derivative” is used to define amino acid sequences (polypeptide), with any insertions, deletions, substitutions and modifications to the amino acid sequences (polypeptide) that do not alter the activity of the original polypeptides. In this connection, a derivative or fragment of the Cas9 protein used by the invention may be any derivative or fragment of the Cas9 protein, specifically as denoted by SEQ ID NO. 4, that do not reduce or alter the activity of the Cas9 protein. By the term “derivative” it is also referred to homologues, variants and analogues thereof. Proteins orthologs or homologues having a sequence homology or identity to the proteins of interest in accordance with the invention, specifically that may share at least 50%, at least 60% and specifically 65%, 70%, 75%, 80%, 85%, 90%, 91%, 92%, 93%, 94%, 95%, 96%, 97%, 98%, 99%, 100%, or higher, specifically as compared to the entire sequence of the proteins of interest in accordance with the invention, for example, the Cas9 protein that comprises the amino acid sequence as denoted by SEQ ID NO. 4. Specifically, homologs that comprise or consists of an amino acid sequence that is identical in at least 50%, at least 60% and specifically 65%, 70%, 75%, 80%, 85%, 90%, 91%, 92%, 93%, 94%, 95%, 96%, 97%, 98%, 99%, or higher to SEQ ID NO. 4, specifically, the entire sequence as denoted by SEQ ID NO. 4.

In some embodiments, derivatives refer to polypeptides, which differ from the polypeptides specifically defined in the present invention by insertions, deletions or substitutions of amino acid residues. It should be appreciated that by the terms “insertion/s”, “deletion/s” or “substitution/s”, as used herein it is meant any addition, deletion or replacement, respectively, of amino acid residues to the polypeptides disclosed by the invention, of between 1 to 50 amino acid residues, between 20 to 1 amino acid residues, and specifically, between 1 to 10 amino acid residues. More particularly, insertion/s, deletion/s or substitution/s may be of any one of 1, 2, 3, 4, 5, 6, 7, 8, 9, or 10 amino acids. It should be noted that the insertion/s, deletion/s or substitution/s encompassed by the invention may occur in any position of the modified peptide, as well as in any of the N′ or C′ termini thereof.

With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention. For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M). Thus, in some embodiments, the invention encompasses Cas9 proteins or any derivatives thereof, specifically a derivative that comprise at least 1, 2, 3, 4, 5, 6, 7, 8, 9, 10 or more conservative substitutions to the amino acid sequences as denoted by SEQ ID NO. 4. More specifically, amino acid “substitutions” are the result of replacing one amino acid with another amino acid having similar structural and/or chemical properties, i.e., conservative amino acid replacements Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q).

Variants of the polypeptides of the invention, specifically, the Cas9 used by the invention, may have at least 80% sequence similarity or identity, often at least 85% sequence similarity or identity, 90% sequence similarity or identity, or at least 95%, 96%, 97%, 98%, or 99% sequence similarity or identity at the amino acid level, with the protein of interest, such as the various polypeptides of the invention. As indicated above, the nucleases, and specifically, the guided nucleases such as Cas9 used by the methods, compositions, cells, kits, uses and systems of the invention may be in some embodiments, catalytically inactive nucleases or any fusion proteins thereof as disclosed by the invention that may enable further manipulation of the level, expression and/or activity of MPO. In such cases, only the targeting properties of these guided nucleases are used (e.g., targeting a target nucleic acid sequence using gRNA), for targeted manipulation of a target sequence. The nucleolytic activity in such cases is undesired. In some embodiments, a guided nuclease with no nucleolytic activity may be used. Thus, in some particular embodiments, the Cas9 enzyme used for the systems of the invention may be a cas9 devoid of any nucleolytic activity, for example, a defective enzyme such as dCas9. dCas9 is a mutant Cas9 that lacks endonucleolytic activity. A non-limiting example for such mutant may be a mutant that carries a point mutation in at least one of D10A (aspartic acid to alanine in position 10) and H840A (histidine to alanine in position 840). Such mutant can be used as a modular RNA-guided platform to recruit different protein effectors to DNA in a highly specific manner in cells (Qi et al., Cell 152: 1173-1183 (2013)). Both repressive and activating effectors can be fused to dCas9 to repress or alternatively, activate gene expression, respectively. Thus, in some embodiments, a fusion protein of dCas9 and activating effectors (e.g., transcription factors or enzymes that perform de methylation) or a fusion of dCas9 with repressors, may be used by the methods, compositions, cells, kits, uses and systems of the invention. More specifically, the activation or repression may be determined by the sgRNAs that recognize the target DNA sequences based only on homologous base pairing. Repression by dCas9 is achieved when the naked mutated protein is guided to the target. This repression is more efficient when the guide targets dCas9 to the promoter region of the desired gene, specifically, the MPO gene, and when the guide sequence is complementary to the non-template strand of the gene. However, this is not essential and in some instances guides to different regions in the gene or the opposite template can also repress efficiently.

Repression by dCas9 can be enhanced by fusing the dCas9 to known repressors. A non-limiting example for such repressor may be the Krüppel associated box (KRAB) domain, which enhances repression of the targets (Gilbert et al., Cell 154:442-451 (2013)). In yet some alternative embodiments, when enhancement of the expression and/or activity of MPO is desired, activation by dCas9 may be achieved when a transcriptional activator is fused to it. A non-limiting example for such activator may be the Herpes simplex virus protein vmw65, also known as VP16 (Gilbert et al., Cell 154:442-451 (2013)). Alternatively, the guide RNAs themselves can be engineered (instead or in addition to dCas9) to recruit either activators or repressors, and thus recruit naked dCas9 and dictate the outcome (Zalatan et al., Cell 160: 339-350 (2015)). For example, the guides can encode an RNA-domain that recruits a specific RNA-binding protein. This RNA-binding protein may be fused to an activator (e.g., VP16) or a repressor (e.g., Krab) and thus the entire recruitment of dCas9 along with the repressor or activator results in a desired outcome, specifically, repression or activation of the MPO gene.

It should be appreciated that when gene editing system is used by the invention to manipulate the levels, expression and or activity of MPO, such system may be delivered either as nucleic acid sequences encoding the components of this system, e.g., constructs comprising nucleic acid sequences that encode Cas9 and the specific gRNAs. However, it should be appreciated that the invention further encompasses in some embodiments thereof the option of using Cas9/gRNA Ribonucleoprotein complexes (Cas9 RNPs), that comprise purified Cas9 and purified gRNAs delivered as functional complexes. In some particular embodiments, purified gRNAs can be generated by PCR amplification of annealed gRNA oligos or in vitro transcription of a linearized gRNA containing plasmid (such as Addgene plasmid 42250). Cas9 (or any variant of Cas9 as discussed herein) can be purified from bacteria through the use of bacterial Cas9 expression plasmids. In yet some further embodiments, the Cas9 RNP delivery to target cells may be carried out in some specific and non-limiting embodiments, via lipid-mediated transfection or electroporation.

As indicated above, the methods, compositions, cells, kits, uses and systems of the invention may further encompass the use of nucleases that cut RNA. Thus, in some embodiments, guided RNA nucleases that may be used by the invention may be CRISPR-Cas systems that target RNA, and can be advantageous (e.g., CRISPR-Cas Type II-A and the newly identified CRISPR-Cas C2c2). As indicated above, the method of the invention may involves the use of either the gene editing system described herein or alternatively, the use of undifferentiated BM cells that display modulated expression and/or activity of MPO. Thus, in some embodiments, when cells are used by the methods of the invention, the undifferentiated BM cell population applicable herein may be a BM cell population transduced or transfected with at least one gene editing compound capable of modulating the expression and/or activity of MPO in these cells. It should be noted that in some embodiments, such undifferentiated BM cell population may be of either an autologous source or alternatively of an allogenic or even syngeneic source. In yet some further alternative embodiments, the methods of the invention may use undifferentiated BM cell population of an allogeneic subject exhibiting an inhibited or eliminated expression and/or activity of MPO. In some embodiments the methods of the invention may use undifferentiated BM cells that were genetically manipulated to eliminate the expression and/or activity of MPO. Thus, in some embodiments, the cells suitable for the methods of the invention may be cells of an autologous source. The term “autologous” when relating to the source of cells, refers to cells derived or transferred from the same subject that is to be treated by the method of the invention. In yet some further embodiments, the cells suitable for the methods of the invention may be cells of an allogenic source. The term “allogenic” when relating to the source of cells, refers to cells derived or transferred from a different subject, referred to herein as a donor, of the same species. More specifically, as indicated above, it should be appreciated that the method of the invention also encompasses the option that the modification of the MPO gene may be performed ex vivo, to cells of the subjects (cells of autologous source) or alternatively, to cells of allogeneic source. In yet some other alternative embodiments, allogeneic undifferentiated BM cells that display modulated expression and/or activity of MPO may be used. The cells, either of autologous or of an allogeneic (or even syngeneic) source, may be than administered to the subject by adoptive transfer. The term “adoptive transfer” as herein defined applies to all the therapies that consist of the transfer of components of the immune system, specifically cells that are already capable of mounting a specific immune response. Still further, in some embodiments, cells suitable in the present application may be immobilized HSP. Hematopoietic stem cells (HSCs) normally reside in the bone marrow but can be forced into the blood, a process termed bone marrow mobilization used to harvest large numbers of HSCs in peripheral blood. One mobilizing agent of choice in accordance with the invention may be granulocyte colony-stimulating factor (G-CSF). The resulting HSCs are further referred as mobilized HSCs.

In case of bone marrow injection, the target cells may be HSPCs and in case of systemic injection, the target cells may be mobilized HSPCs (where the patient is subjected to a preceding treatment of immobilization). It should be noted that both, the bone marrow injection and in some embodiments the systemic injection, of the gene editing compound, may be specifically suitable for in vivo manipulation of MPO in the subject.

The invention provides methods and compositions for modulating and specifically, decreasing the levels, expression and activity of MPO in a subject, specifically, in any tissue or organ of said subject, simply by modifying the expression and/or activity of MPO in undifferentiated BM cells, either in vivo, using the gene editing system of the invention, or ex-vivo by transplanting (e.g. adoptive transfer) BM cells that display modified, or specifically, reduced expression, levels and/or activity of MPO. Thus, as surprisingly shown by the invention, effective modulation of MPO levels and/or activity in specific tissues, for example, brain tissue that may display significant increases of MPO-immunoreactive cells in brain regions affected by neurodegeneration in PD and AD, may be achieved by modulating the MPO levels and/or activity in BM cells. More specifically, in some embodiments the modulation of MPO has been performed in BM cells, provided that said modulation has not been performed in the target brain tissue or any components thereof e.g., astrocytes, microglia and/or neurons. As described herein, the methods of the invention comprise modulating the expression and/or activity of MPO by either administering the at least one gene editing compound/system that is capable of in vivo modulating the MPO expression and/or activity in BM cells, in the treated subject for example a PEN being the CRISPR/Cas9 system described above, or alternatively, by administering undifferentiated BM cells that display modulated expression and/or activity of MPO (either ex vivo manipulation or obtained from an MPO deficient allogenic subject. As also described herein, the term modulating refer to any change or modification such as an increase or decrease of MPO expression and/or activity in relation to the original, non-modulated expression and/or activity of MPO or a control or a normal or a baseline level of MPO expression/activity determined under certain condition.

Thus, in some embodiments, modulating the expression and/or activity of MPO may comprise inhibiting or eliminating the level, the expression and/or activity of MPO in said subject. According to some embodiments, wherein indicated “decreasing” or “inhibiting” the expression or the levels or the activity of MPO, it is meant that such decrease or inhibition may be a decrease or reduction of between about 10% to 100% of the expression, levels, stability and/or activity of MPO. The terms “decreasing” or “inhibiting” as used herein relate to the act of becoming progressively smaller in size, amount, number, or intensity. Particularly, decrease, reduction, elimination, attenuation or inhibition of the MPO expression, levels, stability and/or activity of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100% or more of the expression, level, stability and/or activity of MPO as compared to a suitable control, for example, in cells of a healthy subject, specifically, cells of a healthy subject that display normal levels and/or activity of MPO.

It should be however appreciated, that by offering methods, compositions, cells, kits and systems that modulate the expression and activity of MPO, the invention further encompasses in some alternative embodiments thereof, modulation of the expression and/or activity of MPO that may comprise elevating, establishing or increasing the expression and/or activity of MPO in a subject in need thereof.

According to some embodiments, wherein indicate “increasing” or “enhancing” the expression or the levels or the activity of MPO, it is meant that such increase or enhancement may be an increase or elevation of between about 10% to 100% of the expression and/or stability of MPO. The terms “increase”, “augmentation” and “enhancement” as used herein relate to the act of becoming progressively greater in size, amount, number, or intensity. Particularly, an increase of 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 70%, 800%, 900%, 1000% or more of the expression, level, stability and/or activity of MPO as compared to a suitable control. It should be further noted that increase or elevation may be also an increase of about 2 to 10⁶ folds or more. Still further, it should be appreciated that the increase of the expression, level, stability and/or activity of MPO, may be either in translation or the stability of said MPO, and/or the activity of said MPO. With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively. Therefore, the term increase refers to an increase of about 2, 3, 4, 5, 6, 7, 8, 9, 10, 20, 30, 40, 50, 60, 70, 80, 90, 100, 200, 300, 400, 500, 600, 700, 800, 900, 1000 folds or more.

As indicated above, the invention provides methods, compositions, cells, kits and systems for modulating (e.g., reduce or alternatively, enhance), the expression and/or activity of MPO. MPO expression as used herein may also reflect the stability of the protein. More specifically, “Expression”, as used herein generally refers to the process by which gene-encoded information, specifically, the MPO gene, is converted into the structures present and operating in the cell. Therefore, according to the invention “expression” of a gene, specifically, may refer to transcription into a polynucleotide, translation into a protein, or even posttranslational modification of the protein. Protein stability, as used herein, refers to the physical (thermodynamic) stability, and chemical stability of the protein and relates to the net balance of forces, which determine whether a protein will be in its native folded conformation or a denatured state. More specifically, the levels of proteins, such as MPO within cells are determined not only by rates of synthesis as discussed above, but also by rates of degradation and the half-lives of proteins within cells that vary widely, from minutes to several days. In eukaryotic cells, two major pathways mediate protein degradation, the ubiquitin-proteasome pathway, mentioned herein before, and lysosomal proteolysis.

In yet some further embodiments, the methods of the invention may modulate (e.g., reduce or alternatively, enhance), the activity of MPO. More specifically, MPO activity as used herein may refer in some to any activity performed by MPO, for example the peroxidase activity, that catalyzes the production of reactive intermediates such as hypochlorous acid (HOCl), hypobromous (HOBr), and hypothiocyanous (HOSCN) acids, tyrosyl radical, and reactive nitrogen intermediates from hydrogen peroxide (H₂O₂) and chloride anion (Cr⁻) bromide (Br⁻); thiocyanate (SCN⁻), tyrosine and nitrite, respectively. In yet some further embodiments, MPO activity, may be associated with intracellular killing of bacteria (e.g., Pseudomonas aeruginosa) and fungi (e.g., Candida albicans) by neutrophil via the MPO/HOCl system.

In yet some further embodiments, MPO activity, may also relate to any processes caused by, or mediated directly or indirectly, at least in part, by MPO. As indicated herein before, it should be appreciated that such activities may be mediated either by the hem domain of MPO or by any other domain of MPO. Non-limiting embodiments of such activity relates to NETosis mediated by MPO. More specifically, Neutrophil extracellular traps (NETs) are networks of extracellular fibers, primarily composed of DNA from neutrophils, which bind pathogens. NETs allow neutrophils to kill extracellular pathogens while minimizing damage to the host cells.

NET activation and release, or NETosis, is a dynamic process that can come in two forms, suicidal and vital NETosis. Overall, many of the key components of the process are similar for both types of NETosis, however, there are key differences in stimuli, timing, and ultimate end result Activation pathway—The process begin with NADPH oxidase activation of protein-arginine deiminase 4 (PAD4) via reactive-oxygen species (ROS) intermediaries. PAD4 is responsible for the citrullination of histones in the neutrophil, resulting in decondensation of chromatin. Azurophilic granule proteins such as myeloperoxidase (MPO) and neutrophil elastase (NE) then enter the nucleus and further the decondensation process, resulting in the rupture of the nuclear envelope. The uncondensed chromatin enter the cytoplasm where additional granule and cytoplasmic proteins are added to the early-stage NET. The end result of the process then depends on which NETosis pathway is activated. Suicidal NETosis was first described in a study that noted that the release of NETs resulted in neutrophil-death through a different pathway than apoptosis or necrosis. In suicidal NETosis, the intracellular NET formation is followed by the rupture of the plasma membrane, releasing it into the extracellular space. This NETosis pathway can be initiated through activation of Toll-like Receptors (TLRs), Fc receptors, and complement receptors with various ligands such as antibodies, PMA, and so on. Upon activation of these receptors, downstream signaling results in the release of calcium from the endoplasmic reticulum. This intracellular influx of calcium in turn activates NADPH oxidase, resulting in activation of the NETosis pathway as described above. Vital NETosis can be stimulated by bacterial lipopolysaccharide (LPS), other bacterial products, TLR4-activated platelets, or complement proteins in tandem with TLR2 ligands. Vital NETosis is made possible through the blebbing of the nucleus, resulting in a DNA-filled vesicle that is exocytosed and leaves the plasma membrane intact. Its rapid formation and release does not result in neutrophil death, however, the cell is without DNA, raising questions about whether a cell without DNA can be considered alive. It has been noted that neutrophils can continue to phagocytose and kill microbes after vital NETosis, highlighting the neutrophil's anti-microbial versatility. As detailed above, MPO activity may be mediated via mechanisms that are independent of MPO catalytic activity (“nonenzymatic effect”). In some other embodiments, the term MPO activity refers to at least one of MAPK and NFκB activation, ROS production, surface integrin upregulation, and degranulation, as well as decreased apoptosis leading to enhanced inflammation in the lung. In some specific embodiments, the term MPO activity refers to suppression of apoptosis of human neutrophils. In some embodiments, the term MPO activity encompasses/relates to processes resulting from binding of MPO to CD11b/CD18 integrins. In some embodiments, the MPO activity refers to simulation of polymorphonuclear neutrophils (PMNs) signaling pathways to induce PMN activation. In some other embodiments, the MPO activity refers to MPO's ability to play a role as a survival signal for neutrophils and thereby contribute to prolongation of inflammation. In some other embodiments, the term MPO activity refers endothelial cell activation. In yet some other embodiments, the term MPO activity refers to suppression of Dendritic cells (DCs) function and adaptive immunity. DCs are antigen-presenting cells (also known as accessory cells) of the mammalian immune system, their function being to process antigen material and present it on the cell surface to the T cells of the immune system. DCs act as messengers between the innate and the adaptive immune systems.

In some embodiments, MPO activity refers to an effect on the complement regulatory activity of factor H (FH) via the binding of MPO to FH. It was suggested that MPO-FH interaction may participate in the pathogenesis of (ANCA)-associated vasculitides (AAV) by contributing to activation of the alternative complement pathway. In some embodiments, MPO activity refers to amplification of high glucose-induced endothelial dysfunction in vasculature. In some embodiments, MPO activity refers to binding to human platelets, induction of actin cytoskeleton reorganization and affecting the mechanical stiffness of human platelets. This in turn may result in potentiating store-operated Ca²⁺ entry (SOCE) and agonist-induced human platelet aggregation. In some embodiments, MPO activity refers to reduction in phosphorylation of endothelial NO synthase (eNOS). This may further cause a reduction in NO production, which consequently increases calpain activity by reducing its nitrosylation. In some embodiments, MPO activity refers to binding of MPO to Red blood cells (RBC) membranes.

In yet some further embodiments, MPO activity refers to lipid Peroxidation as indicated herein before, and in some embodiments, specifically to LDL peroxidation. In some embodiments, MPO activity may refer to lipid Carbamylation. In yet some further embodiments, MPO activity may refer to Glycocalyx Binding.

Thus, in some embodiments, the methods, cells, compositions and kits of the invention may modulate, specifically, inhibit, reduce or decrease at least one of the MPO activities specified above, or any combinations thereof. The method of the invention provides modulation, specifically, either decrease or increase of the MPO expression, levels and/or activity in a subject, by applying a gene editing tool to the subject, that specifically leads to in vivo manipulation of MPO expression and/or activity within the undifferentiated BM cells of the subject. Alternatively, or additionally, the subject may be administered with undifferentiated BM cells that display the required modulated expression and/or activity of MPO. It should be noted that the manipulation may occur ex vivo, when BM cells, either of an allogeneic or autologous source are contacted with the editing tool provided by the invention, and upon manipulation of the MPO expression and/or activity, the cells are introduced (in case of autologous cells are re-introduced) to the subject. Introduction of such BM cells to the treated subject results in manipulation of the expression and activity of MPO in the subject. Alternatively, BM cells of an allogeneic subject that display modulated (either increased or decreased) expression and/or activity of MPO, may be introduced to the subject leading to manipulation of MPO in the subject, as also demonstrated by the Examples. Thus, in some embodiments, such manipulation encompasses in vivo (i.e. within a mammalian body) or ex vivo (i.e. in cells removed from the body) manipulation of the expression and/or activity of MPO as further described herein.

In some embodiments, the methods may comprise administration of the at least one gene editing compound to a subject in need thereof, thereby enabling an in vivo manipulation of MPO in undifferentiated BM cells of the subject. It should be noted that such administration methods are known in the art and are further detailed below.

In some other embodiments, the methods comprises ex vivo application. In the context of the present disclosure, ex vivo refers to a step of contacting the at least one gene editing compound or system provided with by the invention with BM cells removed, recovered or obtained from a subject. These cells may be contacted with the gene editing system in conditions suitable for modulating the expression and/or activity of MPO in the cells. The term “contacting” as used herein, means to bring, put, incubate or mix together. As such, a first item is contacted with a second item when the two items are brought or put together, e.g., by touching them to each other or combining them. In the context of the present invention, the term “contacting” includes all measures or steps, which allow interaction between the gene editing compounds of the invention and the cells or, when done in vivo, with the subjects to be modulated. In some embodiments, prior to or following the contacting step, the BM cells may undergo selection to isolate the undifferentiated cells. It should be noted that the step of isolating the undifferentiated BM cells from a BM removed, recovered or obtained from a subject is an optional step. In some embodiments, the BM cell may be contacted with at least one gene editing compound, specifically, any of the gene editing compounds or systems disclosed by the invention. In some embodiments, the isolated undifferentiated BM cell population comprising HSC and/or progenitor cells may be contacted with at least one gene editing compound. In some alternative embodiments, the BM cell are contacted with at least one gene editing compound and the undifferentiated BM cell population comprising HSC and/or progenitor cells are isolated.

In some embodiments, the at least one gene editing compound may be allowed to be in contact with the BM cell population, specifically, the undifferentiated BM cell population for a sufficient time to permit modulation of the MPO gene and to produce modified BM cell population.

Thus, in accordance with some aspects thereof, the present disclosure provides a modified BM cell population, wherein in some embodiments, in such BM cell population, at least part of the cells (e.g., 20% or more, 30% or more, 40% or more, 50%, 60%, 70%, 80%, 90% or more, or even 100%), and preferably, most of the cells, exhibit a modulated expression and/or activity of MPO. In some embodiments, the modified BM cell population may comprise undifferentiated BM cells, specifically, HSC and/or progenitor cells displaying modulated MPO expression and/or activity. In some embodiments, the modified BM cell population may comprise HSC having modulated MPO expression and/or activity. In the present invention, the term “modified BM cells” or “modified BM cell population” is used to denote BM cells that were modified ex-vivo by modulating MPO expression and/or activity.

As indicated above, the invention provides a method for manipulating the immune system of a subject, specifically, by targeting MPO in undifferentiated BM cells of a subject, thereby leading to manipulation of MPO levels in any tissue and organ of said subject. As demonstrated in the next aspect, the invention further uses such modulation for therapeutic applications, specifically for treating MPO-related conditions. It should be therefore appreciated that in some specific and non-limiting embodiments, the invention provides methods, compositions and kits for modulating the levels, expression and/or activity of MPO in a subject by manipulating the immune-system of the subject, specifically, BM cells of the subject to display modulated levels, expression and/or activity of MPO. In other words, by providing a manipulated immune system (either of an autologous or of allogeneic or syngeneic source), or alternatively, by in vivo manipulating the immune system of the subject, using the gene editing system provided herein (e.g., administering the compounds of the gene editing system to the subject), such that the BM cells population of said subject exhibit modulated levels, expression and/or activity of MPO. As such, the methods, kits and compositions of the invention provide a pioneering strategy for modulating expression and/or activity of MPO in any tissue and organ of said subject. This strategy display clear effective therapeutic applications. In yet some further specific embodiments, such manipulation of the immune system of the subject may be achieved by transplanting BM cells that display modulated levels, expression, and/or activity of MPO, to a subject in need, that in some embodiments undergo immune-ablation prior to transplantation. By transplanting BM cells that display modulated (e.g., reduced or eliminated) levels, expression or activity of MPO, the transplanted subject will exhibit modulated expression of MPO in any tissue and organ. It should be understood that as explained in detailed by the invention, the transplanted BM cells may be either autologous BM cells that were obtained from the same subject and ex vivo manipulated using the gene editing systems of the invention to modulate the levels, expression or activity of the MPO. For example, in some specific embodiments, BM cells obtained from the subject may be subjected ex vivo to the gene editing systems of the invention that may result in elimination and reduction of the MPO levels, expression and/or activity. Alternatively, undifferentiated BM cells of an allogeneic or syngeneic source that display (either naturally, or by applying the gene editing systems of the invention) modulated level, expression and/or activity of MPO may be used. Such autologous or allogeneic BM cells that are MPO deficient, may be re-introduced to the subject, that may be treated with immuno-ablating compounds, and thereby these transplanted manipulated BM cells replace the immune-system of a subject. This procedure results in depletion of MPO in any organ or tissue of the subject. It should be understood that the MPO depleted BM cells may be obtained either from the subject, or alternatively from an allogenic subject. More specifically, transplantation of these autologous or allogeneic BM cells to the subject, or in other words replacement of the immune system cells of the subject with the BM cells that display modulated levels, expression and/or activity of MPO (e.g., eliminated or reduced MPO levels, expression and/or activity), leads to modulation of the levels, expression an activity of MPO in every tissue and organ of said subject. MPO plays a role in suppressing the adaptive immune response as demonstrated by two models of enhanced T cell-mediated skin delayed-type hypersensitivity and antigen-induced arthritis in Mpo−/− mice. Mechanistically, MPO released from neutrophils inhibits LPS-induced DC activation as measured by decreased IL-12 production and CD86 expression consequently, limiting T cell proliferation and proinflammatory cytokine production. In contrast, a pathogenic role for MPO in driving autoimmune inflammation was demonstrated using MPO-deficient mice in the K/B×N arthritis and collagen-induced arthritis (CIA) models exhibiting reduced disease severity. Also, increased MPO levels and activity have been observed in many inflammatory conditions and autoimmune diseases including multiple sclerosis (MS) and rheumatoid arthritis (RA). MPO plays a role in modulation of vasculature functioning, associated with chronic vascular diseases such as atherosclerosis. In the extracellular matrix (ECM), MPO works as a nitric oxide(NO)-scavenger consuming NO that leads to impaired endothelial relaxation. MPO and its oxidative species present in the atherothrombotic tissue, promotes lipid peroxidation, conversion of LDL to a highly-uptake atherogenic form, selectively modulates Apolipoprotein A-I (apoA-I) generating dysfunctional HDL particles more susceptible to degradation and impairs the ability of apoA-I to promote cholesterol efflux. Moreover, elevated systemic levels of MPO and its oxidation products are associated with increased cardiovascular risk. As indicated above, MPO has been implicated in variety of pathologic conditions, and thereof modulating the MPO expression provides a specific therapeutic tool for treating and preventing disorders or conditions caused thereby.

The invention further encompasses in some embodiments thereof, methods for modulating MPO expression and/or activity in a subject suffering from at least one MPO-related condition. The method is as described herein above. In yet some further embodiments, the invention also encompasses the use of the methods of modulating MPO levels and/or activity in a subject specifically, as disclosed by the invention, for the treatment and inhibition of at least one MPO-related disorder in a subject in need thereof.

Thus, in a further aspect, the invention provides a method for treating, preventing, ameliorating, inhibiting or delaying the onset of an MPO-related condition in a mammalian subject. More specifically, in some embodiments, the method of the invention may comprise the step of administering to said subject a therapeutically effective amount of at least one of: (a) at least one gene editing compound capable of or adapted for, modulating the expression and/or activity of MPO in at least one undifferentiated BM cell of the subject; and (b) undifferentiated BM cell population exhibiting a modulated expression and/or activity of MPO. It should be understood that the method described herein encompasses treating a subject that suffers from an MPO-related condition with (a), (b) or any combination thereof. Still further, it should be understood that in some aspects thereof, the invention provides a therapeutically effective amount of at least one of: (a) at least one gene editing compound capable of or adapted for, modulating the expression and/or activity of MPO in at least one undifferentiated BM cell of the subject; and (b) undifferentiated BM cell population exhibiting a modulated expression and/or activity of MPO, for use in a method for treating, preventing, ameliorating, inhibiting or delaying the onset of an MPO-related condition in a mammalian subject.

As described herein, the BM cell population and specifically the undifferentiated BM cell population may refer in some embodiments to a population of undifferentiated immature BM cells, specifically HSC in which the expression and/or activity of MPO is modulated. In some embodiments, the undifferentiated BM cell population may be transduced or transfected cell population with at least one gene editing compound capable of modulating the expression and/or activity of MPO in the cells, such that the MPO gene has been modulated.

The undifferentiated BM cell population may be administrated to a subject in need thereof to replace BM cells in a subject having abnormal MPO expression and/or activity, specifically, a subject that display MPO-related conditions. It should be appreciated that in some embodiments, replacement of the BM cell population of the treated subject may involve replacement of about 5%, 10%, 15%, 20%, 25%, 30%, 35%, 40%, 45%, 50%, 55%, 60%, 65%, 70%, 75%, 80%, 85%, 90%, 95%, 97%, 99% or even 100% of the cell population of the subject with BM cells that display modulated expression, level and/or activity of MPO. As appreciated, administration of an undifferentiated BM cell population is denoted herein in some embodiments as a bone marrow transplantation.

In some embodiments, the BM cell population and specifically the undifferentiated BM cell population may be obtained from an autologous source or alternatively, from an allogenic source. In some embodiments, the bone marrow transplant may be an autologous bone marrow transplantation. The term autologous bone marrow transplantation refers to a transplantation in which BM cells are removed, recovered or obtained from a subject and thereafter transplanted into that same subject. In the context of the present disclosure, autologous bone marrow transplantation may comprise in some embodiments the following steps: (a) recovering or obtaining BM cells from a subject; (b) contacting the BM cells with at least one gene editing compound, specifically, under conditions suitable for manipulation of the MPO expression and/or activity; and (c) transplanting (or re-introducing) the BM cells into the same subject. In accordance with some embodiments, step (a) above may be followed by isolating HSC and/or progenitor cells. In accordance with some alternative embodiments, step (b) above may be followed by isolating HSC and/or progenitor cells. In accordance with some specific embodiments, autologous bone marrow transplantation may comprise the following steps: (a) removing, recovering or obtaining BM cells from a subject; (b) isolating undifferentiated cells from the BM cells, such as HSC and/or progenitor cells; (c) contacting the undifferentiated cells with at least one gene editing compound; and (d) transplanting the undifferentiated cells into the same subject. In some embodiments, the bone marrow transplant may be allogeneic bone marrow transplantation. The term allogeneic bone marrow transplantation refers to a transplantation in which BM cells are removed from a subject (referred to herein as a donor) and thereafter transplanted into a different subject (referred to herein as a recipient).

In the context of the present disclosure, allogeneic bone marrow transplantation may comprise the following steps: (a) removing, recovering or obtaining BM cells from a donor; (b) optionally, contacting the BM cells with at least one gene editing compound of the invention; and (c) transplanting the BM cells into a recipient. In accordance with some embodiments, step (a) above may be followed by isolating HSC and/or progenitor cells. In accordance with some alternative embodiments, step (b) above may be followed by isolating HSC and/or progenitor cells. In accordance with some embodiments, allogeneic bone marrow transplantation may comprise the following steps: (a) removing, recovering or obtaining BM cells from a donor subject; (b) isolating undifferentiated cells from the BM cells, such as HSC and/or progenitor cells; optionally (c), contacting the undifferentiated cells with at least one gene editing compound under suitable conditions allowing MPO manipulation; and (d) transplanting the undifferentiated cells into the recipient.

It should be noted that both the autologous bone marrow transplantation and the allogeneic bone marrow transplantation may comprise a step of storing either the BM cells or the undifferentiated BM cells prior to the transplanting step into the same or different subject. In yet another alternative step, the BM cells may be further expended before transplantation.

It should be noted that in some embodiments, the autologous bone marrow transplant as well as the allogeneic bone marrow transplant may be characterized by modulated MPO expression and/or activity compared with the original (unmodulated) MPO expression and/or activity in the transplanted subject. In other words, the transplanted BM cells or the transplanted undifferentiated BM cells are characterized by having a different, modulated MPO expression and/or activity compared with the BM cells or the undifferentiated BM to be replaced by the transplantation process.

The allogeneic bone marrow transplantation according with the present disclosure encompasses transplantation of BM cells obtained from a donor that display modulated MPO expression and/or activity. In some embodiments, the BM cells used for allogeneic bone marrow transplantation by the methods, compositions, kits and systems of the invention, may be undifferentiated BM cells having modulated MPO expression and/or activity obtained by contacting the BM cells with at least one gene editing agent, that is adapted for modulating the expression and/or activity of MPO in the cells. Thus, in some embodiments, the method of allogeneic bone marrow transplantation may comprise removing, recovering or obtaining BM cells from a donor, contacting the cells (optionally undifferentiated cells, specifically, HSC and/or progenitor cells) with at least one gene editing compound to produce modulated BM cells. In accordance with some embodiments, the MPO modulated BM cells may be transplanted into the recipient subject. In yet some alternative embodiments, the BM cells for allogeneic bone marrow transplantation or an allograft, may be BM cell population obtained from a subject exhibiting an abnormal expression and/or activity of MPO (modulated expression and/or activity). These BM cells can be further selected prior to transplantation, to isolate the undifferentiated BM cells. In some specific embodiments, the bone marrow cells used by the methods of the invention for allogeneic bone marrow transplantation may be BM cell population obtained from a subject exhibiting an inhibited or eliminated expression and/or activity of MPO. This will result in decrease or inhibition of MPO expression and/or activity in the transplanted subject. In yet some alternative embodiments, the bone marrow cells for allogeneic bone marrow transplantation may be BM cell population obtained from a subject exhibiting a detectable or enhanced expression and/or activity of MPO. This will result in increase or elevation of MPO expression and/or activity in the transplanted subject.

It should be understood that the invention further encompasses the use of syngeneic undifferentiated BM cells, specifically, of a subject of the same species that is genetically identical, that are optionally manipulated to allow modulated expression and/or activity of MPO. In some embodiments, the BM cells for allogeneic bone marrow transplantation are BM cell population (optionally undifferentiated BM cells) obtained from a subject exhibiting an abnormal expression and/or activity of MPO (modulated expression and/or activity) and may be further modulated by contacting the cells (optionally undifferentiated cells: HSC and/or progenitor cells) with at least one gene editing compound to produce modulated BM cells.

In this connection, it should be noted that the BM cells must display tissue-type compatibility with the recipient. In some embodiments, the method described herein may further comprise, prior to administration, specifically, transplantation of BM cells, application of chemotherapy and/or irradiation and/or use of antibodies and/or chimeric antigen receptor (CAR)-binding ablation and the like, or any combinations thereof, to the recipient subject. In yet some further embodiments, for obtaining BM cells from a donor, the method may further involve the step of immobilization of the cells as discussed herein after.

In the context of the present disclosure, either obtaining or transplantation can be performed by any known methods in the art, for example by bone marrow biopsy and/or bone marrow aspiration. Bone marrow aspiration can be performed under local or general anesthesia. In addition, BM transplantation can be done by any known methods in the art, such as infusion. Still further, in case of bone marrow injection, the target cells may be HSPCs and in case of systemic injection, the target cells may be mobilized HSPCs (where the patient is subjected to a preceding treatment of immobilization). It should be noted that both, the bone marrow injection as well as systemic injection (e.g., intravenous IV), may be specifically suitable for in vivo manipulating and targeting BM cells by the genetic editing compound of the invention, that manipulates MPO expression and/or activity.

In yet some further embodiments, the patient treated by the method of the invention may be subjected to preceding treatment that may involve immunoablation. Treatment of some types of medical conditions, such as cancers, autoimmune diseases and the like often involves an immunoablation to remove the patient's own immune system, for example, prior to transplant of a bone marrow cells graft. Immunoablation can be accomplished by total body radiation or by high dose chemotherapy, use of antibodies, chimeric antigen receptor (CAR)-binding ablation and the like, or any combinations thereof. Therefore, the term “immune ablation” or “immunoablation” refers to the destruction of a patient immune resistance for a medical purpose. Still further, in some embodiments, the patient treated by the method of the invention may be subjected to preceding treatment with steroids. In some embodiments, such treatment may be Steroid administration may be also used to inhibit the immune response. In pharmacologic (supraphysiologic) doses, steroids such as glucocorticoids are used to suppress various allergic, inflammatory, and autoimmune disorders.

As described herein above, the gene editing compound used by the methods, compositions and kits of the invention, may modulate the expression and/or expression of MPO either in vivo in the undifferentiated BM cells of the treated subject or alternatively, ex vivo, in autologous or allogeneic BM cells. In accordance with the methods of the invention, the at least one gene editing compound may comprise at least one of PEN, any nucleic acid molecule comprising a sequence encoding said PEN or any kit, composition or vehicle comprising the at least one PEN, or the nucleic acid sequence encoding such PEN.

In accordance with some embodiments, the at least one PEN may comprise at least one CRISPR/Cas system. In accordance with such embodiments, the method of the invention may comprise the step of administering to said subject an effective amount of: (a) at least one polypeptide comprising at least one Cas protein, or any nucleic acid sequence encoding said Cas protein; and (b) at least one nucleic acid sequence comprising at least one gRNA that targets a protospacer within the MPO gene, or any nucleic acid sequence encoding said gRNA. The method of the invention may use any kit, composition or vehicle comprising at least one of (a) and (b).

As described herein, in some embodiments, the Cas protein applicable for the methods of the invention may be a member of at least one of CRISPR-associated system of Class 1 and Class 2. In some specific embodiments, the Cas protein that may be used by the methods of the invention may be a member of a CRISPR-associated system type II of class 2. In yet some further specific embodiments, the Cas protein may be the Cas9 or any fragments, mutants, variants or derivatives thereof. It should be understood that all gene editing systems described herein before in connection with other aspects of the invention, are also applicable in the present aspect.

In some embodiments, the method of the invention may provide as an element of the CRISP/Cas system, at least one gRNA or crRNA. In accordance with some embodiments, the crRNA may target at least one protospacer comprising the nucleic acid sequence as denoted by any one of SEQ ID NO: 43, SEQ ID NO: 44, SEQ ID NO: 45, SEQ ID NO: 46, as well as SEQ ID NOs. 94, 95, 96, 97, 98, 99, 100, 101, 102, 103, 104, 105, and 106, of the human MPO gene, or any fragments thereof Such crRNA comprise the nucleic acid sequence as denoted by any one of SEQ ID NO: 33, SEQ ID NO: 34, SEQ ID NO: 35, SEQ ID NO: 42, SEQ ID NO: 47, SEQ ID NO: 50, SEQ ID NO: 55, SEQ ID NO: 60, SEQ ID NO: 63, SEQ ID NO: 66, SEQ ID NO: 71, SEQ ID NO: 74, SEQ ID NO: 77, SEQ ID NO: 80, SEQ ID NO: 85, SEQ ID NO: 88 and SEQ ID NO: 91. In yet some further specific embodiments, the gRNA provided by the methods of the invention may target the mouse MPO gene. In some specific embodiments, such gRNAs target protospacers within the mouse MPO that comprise the nucleic acid sequence as denoted by SEQ ID NO:5, SEQ ID NO:6, SEQ ID NO:8, SEQ ID NO:9, SEQ ID NO:10, SEQ ID NO:11, SEQ ID NO:12, SEQ ID NO:13 and SEQ ID NO:14, or any fragments thereof. In yet some further specific embodiments, the gRNA provided by the methods of the invention may comprise the nucleic acid sequence as denoted by SEQ ID NO:5, SEQ ID NO:6, SEQ ID NO:8, SEQ ID NO:9, SEQ ID NO:10, SEQ ID NO:11, SEQ ID NO:12, SEQ ID NO:13 and SEQ ID NO:14.

The therapeutic methods provided by the invention may be specifically applicable for treating subjects suffering from MPO-relate conditions. As used herein, the term MPO-related condition or disease comprises a condition associated with an abnormal, increased, deficient or decreased expression and/or activity of MPO. In other words, the methods described herein are effective in treating a condition associated with a deficient or decreased MPO expression and/or activity or alternatively a condition associated with an increased MPO expression and/or activity. As appreciated, the relative terms “increased”, “deficient”, “reduced” or “decreased” describing MPO expression and/or activity are with respect to a control or a normal or a baseline level of expression/activity determined under certain condition.

It is understood that the interchangeably used terms “associated”, linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. In some specific embodiments, the methods of the invention may be applicable for MPO-related condition that are associated with increased (elevated) MPO expression and/or activity. It should be understood however, that in some embodiments, the methods of the invention may be applicable for subjects that may exhibit normal (not elevated and not reduced) levels of MPO, however, that reduction in the amount of MPO may exhibit beneficial effect on any condition or disorder of said subject that are not necessarily associated with MPO levels. Thus, in some embodiments, modulation of MPO by the methods of the invention may lead to inhibition or decrease in MPO expression and/or activity, thereby alleviating conditions associate with increased or elevated expression of MPO. In some embodiments, the MPO-related condition treatable by the methods of the invention may be at least one of an immune-related disorder, a neurodegenerative disorder, a respiratory disorder, a proliferative disorder, a vascular disorder or any combination thereof.

In some embodiments, the MPO-related condition may be an immune-related disorder. An “Immune-related disorder” or “Immune-mediated disorder”, as used herein encompasses any condition that is associated with the immune system of a subject, more specifically through inhibition or the activation of the immune system, or that can be treated, prevented or ameliorated by reducing degradation of a certain component of the immune response in a subject, such as the adaptive or innate immune response. An immune-related disorder may include infectious condition (e.g., viral infections), metabolic disorders, auto-immune disorders, vasculitis, inflammation and proliferative disorders, specifically, cancer. In some embodiments, the immune-related disorder may be an autoimmune disease. In accordance with some embodiments, the methods of the invention are applicable in treating autoimmune disorders. An autoimmune disease is a condition arising from an abnormal immune response to a normal body part. Examples of an autoimmune disorder include Rheumatoid arthritis (RA), Multiple sclerosis (MS), Systemic lupus erythematosus (lupus), Type 1 diabetes, Psoriasis/psoriatic arthritis, Inflammatory bowel disease including Crohn's disease and Ulcerative colitis, and Vasculitis.

In some specific embodiments, the methods of the invention may be particularly applicable for autoimmune disorder such as multiple sclerosis (MS), Anti-neutrophil cytoplasmic antibodies (ANCAs)-related disorder, and systemic lupus erythematosus (SLE).

In some embodiments, the methods of the invention may be applicable for the treatment of MS and any related conditions or symptoms associated therewith. The term “Multiple Sclerosis” (MS) as herein defined is a chronic inflammatory neurodegenerative disease of the central nervous system that destroys myelin, oligodendrocytes and axons. MS is the most common neurological disease among young adults, typically appearing between the ages of 20 and 40. The symptoms of MS vary, from the appearance of visual disturbance such as visual loss in one eye, double vision to muscle weakness fatigue, pain, numbness, stiffness and unsteadiness, loss of coordination and other symptoms such as tremors, dizziness, slurred speech, trouble swallowing, and emotional disturbances. As the disease progresses patients may lose their ambulation capabilities, may encounter cognitive decline, loss of self-managing of everyday activities and may become severely disabled and dependent.

MS symptoms develop because immune system elements attack the brain's cells, specifically, glia and/or neurons, and damage the protective myelin sheath of axons. The areas in which these attacks occur are called lesions that disrupt the transmission of messages through the brain. Multiple sclerosis is classified into four types, characterized by disease progression: (1) Relapsing-remitting MS (RRMS), which is characterized by relapse (attacks of symptom flare-ups) followed by remission (periods of stabilization and possible recovery; while in some remissions there is full recovery, in other remissions there is partial or no recovery). Symptoms of RRMS may vary from mild to severe, and relapses may last for days or months. More than 80 percent of people who have MS begin with relapsing-remitting cycles; (2) Secondary-progressive MS (SPMS) develops in people who have relapsing-remitting MS. In SPMS, relapses may occur, but there is no remission (stabilization) for a meaningful period of time and the disability progressively worsens; (3) Primary-progressive MS (PPMS), which progresses slowly and steadily from its onset and accounts for less than 20 percent of MS cases. There are no periods of remission, and symptoms generally do not decrease in intensity; and (4) Progressive-relapsing MS (PRMS). In this type of MS, people experience both steadily worsening symptoms and attacks during periods of remission. It should be understood that the method of the invention may be applicable for any type, stage or condition of the MS patient. Treatment using the methods of the invention may result in some embodiments in alleviation of any symptoms, and/or in prolonging the remission period between attacks.

In yet some further embodiments, the methods of the invention may be applicable for the treatment of SLE and any related conditions or symptoms associated therewith. More specifically, Systemic lupus erythematosus (SLE), also known simply as lupus, is an autoimmune disease. Symptoms vary between people and may be mild to severe. Common symptoms include painful and swollen joints, fever, chest pain, hair loss, mouth ulcers, swollen lymph nodes, feeling tired, and a red rash which is most commonly on the face. The disease is characterized by periods of illness, called flares, and periods of remission during which there are few symptoms.

The cause of SLE is not clear, however, is thought to involve genetics together with environmental factors. There are a number of other types of lupus erythematosus including discoid lupus erythematosus, neonatal lupus, and subacute cutaneous lupus erythematosus. It should be appreciated that the invention encompasses each of these types.

Still further, in some embodiments, the methods of the invention may be relevant for other auto immune disorders. For example, for the treatment of ANCA-associated disorders. Anti-neutrophil cytoplasmic antibodies (ANCAs), as used herein, include the perinuclear anti-neutrophil cytoplasmic antibodies (P-ANCA) that target mostly the MPO or EGPA, and are therefore also known as MPO-ANCA, Cytoplasmic anti-neutrophil cytoplasmic antibodies (c-ANCAs), that mostly target the proteinase 3 (PR3) protein and therefore are also known as PR3-ANCA, which is mostly associated with GPA, and atypical ANCA (a-ANCA), also known as x-ANCA, and are a group of autoantibodies, mainly of the IgG type, directed against antigens in the cytoplasm of neutrophil granulocytes (the most common type of white blood cell) and monocytes. p-ANCA is also associated with several medical conditions, it is fairly specific, but not sensitive for ulcerative colitis; a majority of primary sclerosing cholangitis; focal necrotizing and crescentic glomerulonephritis; and rheumatoid arthritis.

Still further, these ANCAs not only activate neutrophils and monocytes by binding membrane-bound PR3/MPO, but interact with the endothelium to enhance the expression of adhesion factors. Although several studies have investigated the ANCA titers in predicting disease relapse during vasculitis remission, the association between ANCA titers and the requirement of permanent dialysis in the active phase of AAV has not been well documented. In Asian countries, MPO-ANCA associated vasculitis is more common than PR3-ANCA associated vasculitis. They are detected as a blood test in a number of autoimmune disorders, but are particularly associated with systemic vasculitis, so called ANCA-associated vasculitides (AAV) including granulomatosis with polyangiitis, microscopic polyangiitis, primary pauci-immune necrotizing crescentic glomerulonephritis (a type of renal-limited microscopic polyangiitis), eosinophilic granulomatosis with polyangiitis (previously known as Churg-Strauss syndrome) and drug induced vasculitides. PR3 directed c-ANCA is present in 80-90% of granulomatosis with polyangiitis, 20-40% of microscopic polyangiitis, 20-40% of pauci-immune crescentic glomerulonephritis and 35% of eosinophilic granulomatosis with polyangiitis. c-ANCA (atypical) is present in 80% of cystic fibrosis (with BPI as the target antigen) and also in inflammatory bowel disease, primary sclerosing cholangitis and rheumatoid arthritis (with antibodies to multiple antigenic targets). p-ANCA with MPO specificity is found in 50% of microscopic polyangiitis, 50% of primary pauci-immune necrotizing crescentic glomerulonephritis and 35% of eosinophilic granulomatosis with polyangiitis. p-ANCA with specificity to other antigens are associated with inflammatory bowel disease, rheumatoid arthritis, drug-induced vasculitis, autoimmune liver disease, drug induced syndromes and parasitic infections. Atypical ANCA is associated with drug-induced systemic vasculitis, inflammatory bowel disease and rheumatoid arthritis.

More specifically, Antineutrophil cytoplasmic antoantibody (ANCA)-Associated Vasculitis (AVV) is a systemic disease characterized by inflammation and necrosis of small vessels, resulting in multiple organ damage and dysfunction. The kidney is the most frequently affected organ, leading to pauci-immune segmental necrotizing crescentic glomerulonephritis, which is called ANCA-associated glomerulonephritis (AAGN).

Clinical and experimental evidence indicate that ANCA causes pauci-immune necrotizing, crescentic glomerulonephritis (NCGN) and systemic small vessel vasculitis in humans.

In some specific embodiments, the methods, of the invention, as well as the compositions, cells and kits disclosed by the invention may be applicable for P-ANCA associated disorders.

More specifically, one of the major target antigens for Antineutrophil cytoplasmic antoantibodies is MPO. MPO-ANCA do not target a single epitope, but rather a small number of regions of MPO, primarily in the carboxy-terminus of the heavy chain. Thus, by eliminating the expression, levels and/or activity of MPO, the methods, composition, cells and kits of the invention address the variability of the autoantibody targeted epitopes in the MPO.

In some embodiments, the methods of the invention may be applicable to P-ANCA associated disorder that is Anca-associated vasculitis. AAV, is classified into four categories depending on the clinical features: microscopic polyangiitis (MPA), granulomatosis with polyangiitis (GPA), eosinophilic GPA (EGPA) and renal-limited vasculitis (RLV). Organ involvement produces many alterations in various categories of AAV, and the clinical manifestations lack specificity. At a population level, little is known about the epidemiology and outcome of pauci-immune GN. PICG represents up to 80% of cases of Rapidly progressive glomerulonephritis (RPGN), the incidence of which is estimated to be 7-10 cases per million people per year in the United States. Pauci-immune GN (PICG) has a predilection for whites compared to blacks, with roughly equal representation in men and women.

Without treatment, Pauci-immune GN-PICG has a 1-year mortality of 80%. With aggressive immunosuppression, however, the 5-year survival is up to 75%. Older age, dialysis dependency, and pulmonary hemorrhage all worsen the chances of survival. For instance, irreversible dialysis-dependent renal failure lowers the 5-year survival rate to 35%. From a renal outcome standpoint, about 25% of patients progress to Kidney failure, also called end-stage renal disease (ESRD), which is the last stage of chronic kidney disease.

Thus in some embodiments, ANCA-related or associated disorders include, but are not limited to ANCA-associated vasculitides (AAV), ANCA-associated glomerulonephritis (AAGN), crescentic glomerulonephritis (NCGN), and Rapidly progressive glomerulonephritis (RPGN). Moreover, the methods, compositions, cells and kits of the invention are applicable in some embodiments for the treatment of any of the P-ANCA-related disorders disclosed herein.

In some further embodiments, the methods of the invention may be applicable for treating immune-related disorder such as an inflammatory disorder. In accordance with some embodiments, the methods of the invention are applicable in treating an inflammatory disorder. The terms “inflammatory disease” or “inflammatory-associated condition” refers to any disease or pathologically condition which can benefit from the reduction of at least one inflammatory parameter, for example, induction of an inflammatory cytokine such as IFN-gamma and IL-2 and reduction in IL-6 levels. The condition may be caused (primarily) from inflammation, or inflammation may be one of the manifestations of the diseases caused by another physiological cause. In some embodiments, an inflammatory disease that may be applicable for the methods of the invention may be any one of atherosclerosis, Rheumatoid arthritis (RA) and inflammatory bowel disease (IBD). As disclosed by the Examples, the present invention shows that MPO-deficient 5×FAD exhibit superior behavioral performance compared to MPO-expressing 5×FAD mice, thus reflecting limited cognitive decline. The invention also show lower inflammatory response in the brains of these mice, as reflected by reduced levels of astrocytic activation, APOE expression, and the levels of several pro-inflammatory cytokines.

Thus, in certain embodiments, the invention provides methods, compositions, cells and kits for inhibiting or reducing cognitive decline in a subject in need. In yet some further embodiments, the invention provides methods, compositions, cells and kits for inhibiting or reducing inflammatory response in the brains of a subject in need thereof, specifically, a subject suffering from AD. Still further, in some embodiments, the invention provides methods, compositions, cells and kits for inhibiting or reducing levels of astrocytic activation, in a subject in need. In yet some further embodiments, the invention provides methods, compositions, cells and kits for inhibiting or reducing APOE expression in a subject in need. Still further, in some embodiments, the invention provides methods, compositions, cells and kits for inhibiting or reducing the levels of several pro-inflammatory cytokines in a subject in need. Such cytokines may be in some embodiments at least one of IL-1 beta, CXCL10, CCL2 and TNF alpha. In some embodiments such subject may be a subject suffering from a neurodegenerative disorder. In yet some further specific embodiments, such neurodegenerative disorder may be AD.

Still further, Central to neutrophil function, and longtime suspected in its role in neurodegeneration, MPO stands as a prominent mediator of neutrophils damage in AD. Indeed, several associations of MPO with AD have been reported in the past. It was previously shown that polymorphism in the 463G/A loci in the promoter of human MPO, linked to increased MPO expression, elevated the risk of developing late onset AD. Furthermore, an interaction was identified between MPO and the APOE c4 allele, the most common genetic risk factor for late onset AD. Following studies, however, exhibited contradicting findings regarding the association of the 463G/A polymorphism with AD. Green and colleagues have reported elevated MPO immunoreactivity and activity in the cortices of AD patients, along with increased prevalence of its oxidation products. However, MPO expression in that study was attributed to neurons. Reynolds and colleagues, detected MPO expression in CD68 expressing cells surrounding amyloid-β plaques, therefore attributing it to microglia/macrophages. More recently, MPO expression was reported in association with amyloid-β plaques in the brains of AD patients. Interestingly, no MPO mRNA expression was detected in those brains, indicating that the origin of MPO observed is of peripheral, myeloid nature. While these observations provide compelling evidence to the involvement of MPO in AD pathogenesis, to the inventors knowledge, the present invention is the first to examine the effect of MPO deficiency in an experimental model of AD. The present invention is focused on peripheral MPO, predominantly derived from neutrophils by utilizing hematopoietic depletion and repopulation methods, to establish MPO deficiency in peripheral myeloid cells, without affecting CNS-derived MPO. Oxidative stress and vascular damage are considered key events in the pathogenesis of AD. These can potentially lead to blood brain barriers dysfunction, decreased cerebral blood flow, and substantial oxidative and inflammatory damage within the CNS. Recent implications for neutrophil function in neurodegeneration include hyper-activation and senescence, enhanced adhesion in cortical capillaries, extravasation and degranulation in the brain parenchyma and the formation of NETs, leading to profound inflammatory response. The data disclosed by the invention also demonstrate that hematological MPO deficiency limit the inflammatory response. In addition, the profound protection against cognitive decline following MPO depletion may account for the reduced oxidative damage derived from neutrophils, which consider a substantial part of the pathology in AD. In this context, the results disclosed herein are in line with recent publications by Zenaro et al. and by Cruz-Hernandez et al. exhibiting functional protection in mouse models of AD following neutrophil depletion (Zenaro, E. et al. Neutrophils promote Alzheimer's disease-like pathology and cognitive decline via LFA-1 integrin. Nat. Med. 21, 880-886 (2015), and Cruz Hernandez, J. C. et al. Neutrophil adhesion in brain capillaries reduces cortical blood flow and impairs memory function in Alzheimer's disease mouse models. Nat. Neurosci. (2019). doi:10.1038/s41593-018-0329-4).

MPO function have been implicated in endothelial dysfunction and vascular damage in the context of cardiovascular and kidney diseases. Positively charged MPO secreted by activated neutrophils accumulates along the endothelium and subendothelial space, through its interaction with anionic glycocalyx. While oxidants derived from bound MPO were shown to reduce glycocalyx thickness and damage to the endothelium, MPO was also shown to promote neutrophil recruitment and transmigration. This may result in amplified MPO response, and in situ accumulation of neutrophils. Interestingly, the inventors found a decrease in VCAM1 expression in hippocampal samples of MPO deficient 5×FAD mice, suggesting an increase in inflammation in the endothelium. However, the effect of MPO deficiency on recruitment of neutrophils in microvascular endothelium and its effects on capillary stalls and cerebral blood flow, and neutrophil migration, are yet to be determined.

The results presented by the invention clearly indicate that neutrophil-derived MPO plays a central role in the AD-like pathogenesis in 5×FAD mice. These results propose MPO inhibition as a novel therapeutic target for the treatment of AD.

Thus, in yet some further embodiments, the MPO-related condition may be a neurodegenerative disorder. In some specific embodiments, the methods of the invention are applicable in treating a neurodegenerative disorder. In some embodiments, the neurodegenerative disorder may further involve inflammatory and/or vascular causes.

Neurodegeneration is the umbrella term for the progressive loss of structure or function of neurons, including synaptic dysfunction and death of neurons. Many neurodegenerative diseases including Alzheimer's and Parkinson's are associated with neurodegenerative processes. Other examples of neurodegeneration that may be also applicable herein may include Friedreich's ataxia, Lewy body disease, spinal muscular atrophy, multiple sclerosis, frontotemporal dementia, corticobasal degeneration, progressive supranuclear palsy, multiple system atrophy, hereditary spastic paraparesis, amyloidosis, Amyotrophic lateral sclerosis (ALS), and Charcot Marie Tooth. It should not be overlooked that normal aging processes include progressive neurodegeneration, specifically, age-related cognitive decline (ACD) and mild cognitive impairment (MCI) are also applicable in the present invention. In yet some further embodiments, the methods, kits, compositions and cells of the invention may be applicable for Duchenne muscular dystrophy (DMD), or any conditions associated therewith. In yet some further embodiments, the methods, kits, compositions and cells of the invention may be applicable for Becker muscular dystrophy. In yet some further embodiments, the methods, kits, compositions and cells of the invention may be applicable for Tuberous sclerosis complex (TSC).

In more specific embodiments, the methods of the invention may be applicable for treating a neurodegenerative disorder such as Alzheimer's disease or Parkinson's disease.

Alzheimer's disease (AD), as used herein refers to a disorder that involves deterioration of memory and other cognitive domains that in general leads to death within 3 to 9 years after diagnosis. The principal risk factor for Alzheimer's disease is age. The incidence of the disease doubles every 5 years after 65 years of age. Up to 5% of people with the disease have early onset AD (also known as younger-onset), that may appear at 40 or 50 years of age. Many molecular lesions have been detected in Alzheimer's disease, but the overarching theme to emerge from the data is that an accumulation of misfolded proteins in the aging brain results in oxidative and inflammatory damage, which in turn leads to energy failure and synaptic dysfunction. More specifically, accumulation of Aβ within has been shown in structurally damaged mitochondria isolated from the brains of patients with Alzheimer's disease.

Alzheimer's disease may be primarily a disorder of synaptic failure. Hippocampal synapses begin to decline in patients with mild cognitive impairment (a limited cognitive deficit often preceding dementia) in whom remaining synaptic profiles show compensatory increases in size. In mild Alzheimer's disease, there is a reduction of about 25% in the presynaptic vesicle protein synaptophysin. With advancing disease, synapses are disproportionately lost relative to neurons, and this loss is the best correlate with dementia. Aging itself causes synaptic loss, which particularly affects the dentate region of the hippocampus.

There is no single linear known chain of events or pathways that could initiate and drive Alzheimer's disease. AD is a progressive disease, where dementia symptoms gradually worsen over a number of years. In its early stages, memory loss is mild, but with late-stage AD, individuals lose the ability to carry on a conversation and respond to their environment. Those with AD live an average of eight years after their symptoms become noticeable to others, but survival can range up to 20 years, depending on age and other health conditions.

The most common early symptom of AD is difficulty remembering newly learned information because AD changes typically begin in the part of the brain that affects learning and memory. As AD advances through the brain it leads to increasingly severe symptoms, including disorientation, mood and behavior changes; deepening confusion about events, time and place; unfounded suspicions about family, friends and professional caregivers; more serious memory loss and behavior changes; and difficulty speaking, swallowing and walking.

The National Institute of Neurological and Communicative Disorders and Stroke (NINCDS) and the Alzheimer's Disease and Related Disorders Association (ADRDA, now known as the Alzheimer's Association) established the most commonly used NINCDS-ADRDA Alzheimer's Criteria for diagnosis in 1984, extensively updated in 2007. These criteria require that the presence of cognitive impairment, and a suspected dementia syndrome, be confirmed by neuropsychological testing for a clinical diagnosis of possible or probable AD. A histopathologic confirmation including a microscopic examination of brain tissue is required for a definitive diagnosis. Good statistical reliability and validity have been shown between the diagnostic criteria and definitive histopathological confirmation. Eight cognitive domains are most commonly impaired in AD: memory, language, perceptual skills, attention, constructive abilities, orientation, problem solving and functional abilities. These domains are equivalent to the NINCDS-ADRDA Alzheimer's Criteria as listed in the Diagnostic and Statistical Manual of Mental Disorders (DSM-IV-TR) published by the American Psychiatric Association. Beside symptomatic treatments to temporarily slow the worsening of dementia symptoms, AD has no current cure, and the current treatments cannot stop AD from progressing. It should be understood that the methods of the invention as well as the compositions, cells, kits, uses and systems provided by the invention may be applicable for any stage, condition or symptom associated with AD, of any of the MPO-related conditions discussed herein. It should be further appreciated that in some embodiments, the methods, compositions, cells and kits of the invention may be applicable for any stage, type, symptom or degree of the disease in AD patients.

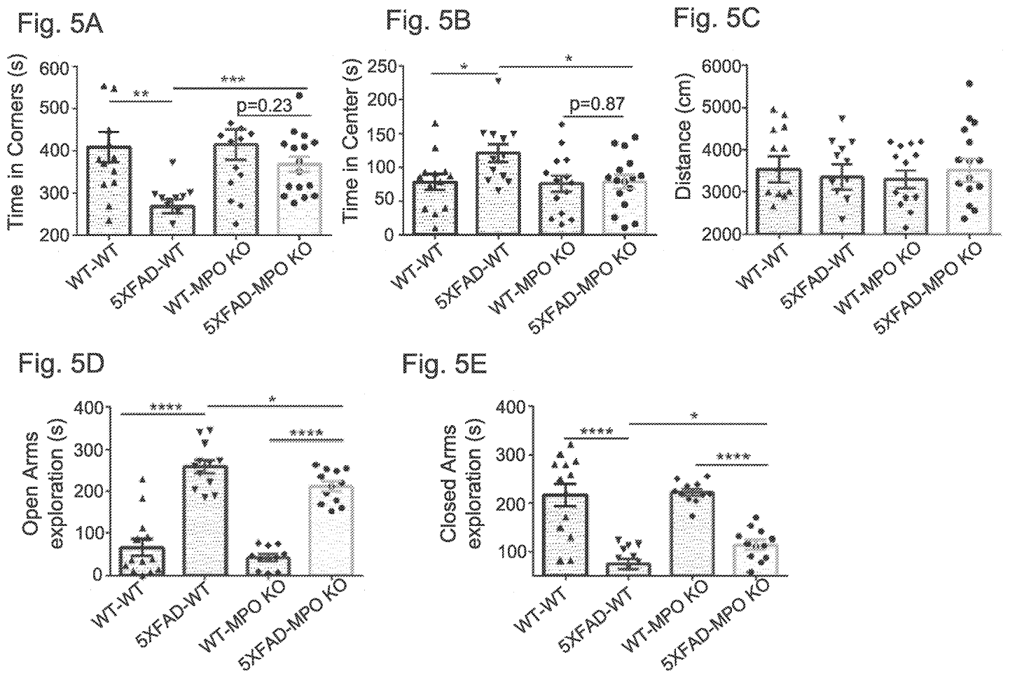

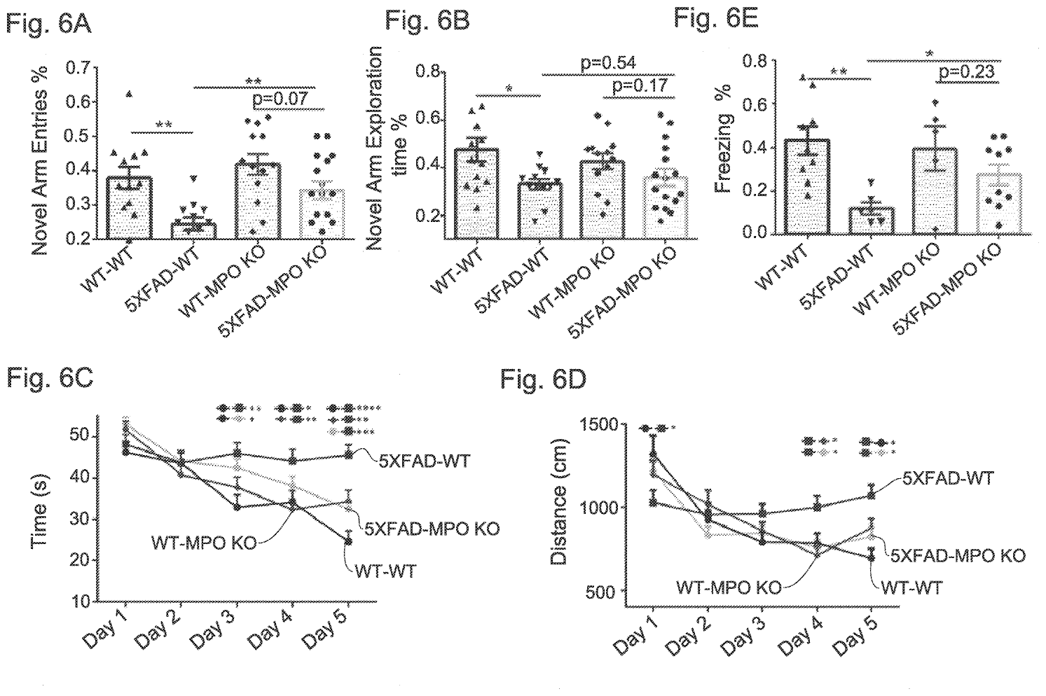

As shown by FIGS. 5 and 6, adoptive transfer of MPO-KO BM cells to AD animals (5×FAD), clearly improved spatial learning and memory, associative learning and anxiety/risk assessment behavior. Thus, in some embodiments, the methods, compositions, cells and kits of the invention may be applicable in improving and enhancing spatial learning in AD patients. In yet some further embodiments, the methods, compositions, cells and kits of the invention may be applicable in improving and enhancing memory in AD patients. Still further, in some embodiments, the methods, compositions, cells and kits of the invention may be applicable in improving and enhancing associative learning in AD patients. In some further embodiments, the methods, compositions, cells and kits of the invention may be applicable in improving and enhancing anxiety/risk assessment behavior in AD patients.

As indicated above, AD is considered as a condition involving protein misfolding (e.g., Aβ peptide). Thus, in yet some further embodiments, the methods, compositions, systems, kits and uses of the invention may be applicable for any condition associated with protein misfolding, for example, synucleinopathies and tauopathies. More specifically, Alpha-synuclein pathology disorders” or “Synucleinopathies” is used to name a group of neurodegenerative disorders characterized by fibrillary aggregates of alpha-synuclein protein in the cytoplasm of selective populations of neurons and glia. Alpha-synuclein aggregates comprising monomeric, oligomeric intermediate, or fibrillar forms are thought to be involved in a critical step in the pathogenesis of Parkinson's disease (PD) and in other alpha-synucleinopathies, such as multiple system atrophy (MSA) and dementia with Lewy bodies (DLB). Thus, in some embodiments the methods, compositions, systems, kits and uses of the invention may be applicable for any of the synucleinopathies disclosed herein. Still further, in some embodiments the methods, compositions, systems, kits and uses of the invention may be applicable for any condition associated with accumulation of Tau protein. More specifically, “Tau protein” as used herein, refers to neurofibrillary tangles, which are filamentous inclusions in pyramidal neurons, characteristic for Alzheimer's disease and other neurodegenerative disorders termed tauopathies. Elucidation of the mechanisms of their formation may provide targets for future therapies. Accumulation of hyperphosphorylated Tau protein as paired helical filaments in pyramidal neurons is a major hallmark of Alzheimer disease. Besides hyperphosphorylation, other modifications of the Tau protein, such as cross-linking, are likely to contribute to the characteristic features of paired helical filaments, including their insolubility and resistance against proteolytic degradation. These neurofibrillary tangles, consist of hyperphosphorylated and aggregated forms of the microtubule-associated protein tau.

As shown in Example 2, adoptive transfer of undifferentiated MPO KO BM cells led to clear inhibition of cognitive impairment in AD and improved cognitive functions and associative learning. Therefore, in some embodiments, the methods compositions, systems, kits and uses of the invention may be applicable for any condition associated with cognitive decline or impairment. In some embodiments, the invention may be applicable for MCI, “Age-associated mild cognitive impairment (MCI)”, as used herein is a condition that causes cognitive changes. MCI that primarily affects memory may be classified as “amnestic MCI” where the subjects experience impairment in memorizing information that relate to recent events, appointments or conversations or recent events. MCI that affects thinking skills other than memory is known as “nonamnestic MCI”. Thinking skills that may be affected by nonamnestic MCI include the ability to make sound decisions, judge the time or sequence of steps needed to complete a complex task, or visual perception.

Normal aging is associated with a decline in various memory abilities in many cognitive tasks; the phenomenon is known as age-related memory impairment (AMI), age-associated memory impairment (AAMI) or age-associated cognitive decline (ACD). The ability to encode new memories of events or facts and working memory shows decline in both cross-sectional and longitudinal studies. Studies comparing the effects of aging on episodic memory, semantic memory, short-term memory and priming revealed that episodic memory is especially impaired in normal aging; some types of short-term memory are also impaired. The deficits may be related to impairments seen in the ability to refresh recently processed information. Normally, there is little age-associated decline in some mental functions such as verbal ability, some numerical abilities and general knowledge but other mental capabilities decline from middle age onwards, or even earlier. The latter include aspects of memory, executive functions, processing speed and reasoning. It should be therefore appreciated that in some embodiments, the invention provides methods and compositions for the treatment for any cognitive decline, specifically cognitive decline associated with age, specifically, the age of 50, 55, 60, 65, 70, 75, 80, 85, 90, 95 and more, years of age.

In yet some further embodiments, the methods compositions, systems, kits and uses of the invention may be applicable for DLB. “Dementia with Lewy Bodies (DLB)”, as used herein, is a relatively common cause of dementia, estimated to account for up to 30% of dementia cases, and affecting up to 5% of those over the age of 75. Pathologically, it is defined by the presence of alpha synuclein containing Lewy bodies in the brain, but their distribution, affecting the neocortex, limbic system and brainstem. Clinically, DLB is characterized by a progressive dementia with prominent visual hallucinations and delusions, and parkinsonism with bradykinesia and rigidity but typically minimal tremor. Marked cognitive fluctuations are a common feature of this condition, with episodes of confusion, excessive somnolence, and incoherent speech which can revert to a near normal state within hours.

In some further embodiments, the methods compositions, systems, kits and uses of the invention may be applicable for PD. “Parkinson's disease (PD)” as used herein, is a neurodegenerative disease resulting from degeneration of midbrain dopamine neurons and accumulation of alpha-synuclein containing Lewy bodies in surviving neurons. The diagnosis of PD is based on the presence of cardinal motor features in the absence of other aetiological conditions. These motor features include the classical triad of bradykinesia, a resting pill-rolling tremor, and rigidity typically in association with hypomimia, hypophonia, micrographia and postural instability. Non-motor features of PD may even precede its diagnosis, constituting prodromal or premotor PD. These premotor features include problems with olfaction, constipation, mood and sleep, and following the clinical diagnosis of PD, they can become more prominent. Cognitive problems and dementia also commonly develop in PD, affecting almost 50% by 10 years from diagnosis. However, in some individuals with an alpha-synucleinopathy, significant cognitive problems precede the onset of parkinsonian motor symptoms, and these cases are clinically classified with a diagnosis of Dementia with Lewy Bodies. There is clearly a major degree of overlap between these two conditions both clinically and pathologically, but at present, the clinical distinction rests on the time interval between the onset of motor symptoms and dementia, with a minimum one year interval being required for a diagnosis of PD as opposed to Lewy body dementia (DLB). Still further, in some embodiments, the methods compositions, systems, kits and uses of the invention may be applicable for MSA. “Multiple system atrophy (MSA)”, is a neuropathology that includes cell loss and gliosis in nigrostriatal and olivopontocerebellar structures taking the form of glial cytoplasmic inclusions containing fibrillar alpha-synuclein within oligodendrocytes. It presents with autonomic dysfunction along with parkinsonism and cerebellar dysfunction in varying combinations, and is clinically classified as being either mainly cerebellar in its presentation (MSA-C) or mainly parkinsonian (MSA-P).

Still further, as indicated above, neurodegenerative disorders are clearly associated with vascular and ischemic conditions. Thus, in some embodiments, the methods, compositions, systems, kits and uses of the invention may be applicable for any vascular and/or ischemic condition. More specifically, brain ischemia (or cerebral ischemia, cerebrovascular ischemia) is a condition in which there is insufficient blood flow to the brain to meet metabolic demand. This leads to poor oxygen supply or cerebral hypoxia and thus to the death of brain tissue or cerebral infarction/ischemic stroke. It is a sub-type of stroke along with subarachnoid hemorrhage and intracerebral hemorrhage. Ischemia leads to alterations in brain metabolism, reduction in metabolic rates, and energy crisis. There are two types of ischemia: focal ischemia, which is confined to a specific region of the brain; and global ischemia, which encompasses wide areas of brain tissue.

The main symptoms involve impairments in vision, body movement, and speaking. The causes of brain ischemia vary from sickle cell anemia to congenital heart defects. Symptoms of brain ischemia can include unconsciousness, blindness, problems with coordination, and weakness in the body. Other effects that may result from brain ischemia are stroke, cardiorespiratory arrest, and irreversible brain damage.

In an ischemic stroke, blood supply to part of the brain is decreased, leading to dysfunction of the brain tissue in that area. There are four major causes for ischemic stroke, thrombosis (obstruction of a blood vessel by a blood clot forming locally), embolism (obstruction due to an embolus from elsewhere in the body), systemic hypoperfusion (general decrease in blood supply, e.g., in shock); and cerebral venous sinus thrombosis.

There are various classification systems for acute ischemic stroke. The Oxford Community Stroke Project classification (OCSP, also known as the Bamford or Oxford classification) relies primarily on the initial symptoms; based on the extent of the symptoms, the stroke episode is classified as total anterior circulation infarct (TACI), partial anterior circulation infarct (PACI), lacunar infarct (LACI) or posterior circulation infarct (POCI). These four entities predict the extent of the stroke, the area of the brain that is affected, the underlying cause, and the prognosis. The TOAST (Trial of Org 10172 in Acute Stroke Treatment) classification is based on clinical symptoms as well as results of further investigations; on this basis, a stroke is classified as being due to (1) thrombosis or embolism due to atherosclerosis of a large artery, (2) an embolism originating in the heart, (3) complete blockage of a small blood vessel, (4) other determined cause, (5) undetermined cause (two possible causes, no cause identified, or incomplete investigation)

Small chronic intracerebral hemorrhages (<5 to 10 mm in diameter) are termed cerebral microhemorrhages (CMHs; also described as microbleeds, multifocal signal loss lesions, petechial hemorrhages). CMHs are due to the rupture of small arteries, arterioles, and/or capillaries (in human CMHs, the diameter of ruptured vessels is estimated to be less than 200 μm, with many bleeds occurring at the arteriole and capillary levels). CMHs appear as small, oval hypointense lesions corresponding to focal hemosiderin depositions, which can be best detected using T2*-weighted Gradient-Recall Echo (T2*-GRE) MRI sequences.

Age is the most significant independent risk factor for CMHs. Prevalence of CMHs is low in younger subjects and progressively increases with age. The majority of studies report that the prevalence of CMHs is 24% to 56% in elderly patients. Hypertension is another major independent risk factor for CMHs. Cerebral amyloid angiopathy and Alzheimer's disease (AD) also constitute an important risk factor for CMHs. In type 1 diabetic patients with proliferative diabetic retinopathy, an increased prevalence of CMHs was reported, suggesting that generalized microangiopathy may contribute to both the cerebral and retinal microvascular injury. CMHs are also found in close to half of individuals affected by cerebral autosomal-dominant arteriopathy with subcortical infarcts and leukoencephalopathy (CADASIL), which is an adult-onset hereditary stroke disorder caused by mutations of the Notch 3 gene.

Still further, in some embodiments, the methods, compositions, systems, kits and uses of the invention may be applicable for cSVD. Cerebral small vessel disease (cSVD) is a term used for different pathological processes that affect the small vessels of the brain, including small arteries, arterioles, capillaries, and small veins. cSVD has a crucial role in lacunar cerebral infarction and deep or cortical haemorrhages. In addition to cognitive decline and dementia, gait problems are also frequently associated with cSVD. Approximately one-fifth of symptomatic strokes are lacunar stroke syndromes, which are often the more severe kind of strokes namely spontaneous parenchymal brain hemorrhage (PBH). These are associated with cSVD. However, even though sporadic cSVD is the leading cause of PBH, the topography of the underlying microvascular pathology is different in each case. cSVD is categorized in two main forms. The first is the amyloidal form which includes cerebral amyloid angiopathy (CAA), a chronic degenerative disease. The second form is characterized as non-amyloidal form of cSVD which is often related to common vascular risk factors, such as elderly age, hypertension, diabetes mellitus, and many other factors.

Cerebral amyloid angiopathy (CAA), also known as congophilic angiopathy, is a form of angiopathy in which amyloid deposits form in the walls of the blood vessels of the central nervous system. The term congophilic is used because the presence of the abnormal aggregations of amyloid can be demonstrated by microscopic examination of brain tissue after application of a special stain called Congo red. The amyloid material is only found in the brain and as such the disease is not related to other forms of amyloidosis.

CAA is a common amyloidal form of cSVD. Incidences of CAA are mostly associated with advanced age. It is caused by a progressive deposition of β-amyloid in the walls of cortical and leptomeningeal small arteries, which leads to vessel dysfunction and brain parenchymal injury. Deposition of β-amyloid is thought to be involved in vascular occlusion and rupture. CAA related vasculopathy includes features such as fibrinoid necrosis, loss of smooth muscle cells, wall thickening, microaneurysm formation, and perivascular blood breakdown with the resulting product deposition. Based on the specific location of amyloid deposition and allelic difference, at least two pathological subtypes of CAA have been identified: CAA type-1 characterized by amyloid in cortical capillaries, and CAA type-2, in which amyloid deposits are restricted to leptomeningeal and cortical arteries, but not capillaries. Decreased cortical grey matter in the occipital lobe and decreased flux in the basilar artery were noted in patients of symptomatic hereditary cerebral hemorrhage with amyloidosis-Dutch type (HCHWA-D). The appearance of cortical thinning in patients with HCHWA-D indicated that vascular amyloid is an independent contributor to cortical atrophy. CAA-related cortical atrophy was facilitated by vascular dysfunction and even observed in the absence of Alzheimer's disease. Moreover, apolipoprotein E (APOE) gene polymorphism is associated with the two subtypes of CAA. APOE ε4 allele and APOE ε2 is legitimately associated with type-1 and type-2 diseases, respectively.

It should be therefore appreciated that the methods, compositions, systems, kits and uses of the invention may be applicable for any ischemic conditions, specifically, any of the conditions disclosed herein.

Still further, neutrophils have long been known as innate immune cells that phagocytose and kill pathogens and mount inflammatory responses protecting the host from infection. The formation of neutrophil extracellular DNA traps (NETs) has defined new roles for neutrophils in inflammation and immunity but also in pathological conditions including cancer biology and thrombosis, as well as associated conditions (e.g., Trousseau syndrome). The release of the neutrophil's chromatin has been shown as affecting different steps of tumor development including tumor growth, angiogenesis, metastasis and immune suppression. As indicated above, MPO is involved in NETs formation in neutrophils. Therefore, by modulating, and specifically, inhibiting MPO expression, levels and/or activity, the invention provides a strategy for modulating and inhibiting NET formation in a subject, and thereby offers an approach for treating cancer and associated disorders. Thus, in some embodiments, the MPO-related condition that may involve an enhanced expression or activity of MPO, may be a proliferative disorder. Thus, in some specific embodiments, the methods of the invention may be applicable in treating a proliferative disorder. The proliferative disorder according with some embodiments of the present invention may be cancer and/or any related pathology such as metastasis. As used herein, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods and compositions of the present invention may be used in the treatment of non-solid and solid tumors. Malignancy, as contemplated in the present invention may be any one of carcinomas, melanomas, lymphomas, leukemias, myeloma and sarcomas. Carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges. Melanoma as used herein, is a malignant tumor of melanocytes. Melanocytes are cells that produce the dark pigment, melanin, which is responsible for the color of skin. They predominantly occur in skin, but are also found in other parts of the body, including the bowel and the eye. Melanoma can occur in any part of the body that contains melanocytes.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas. Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered. Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

Further non-limiting examples for malignancies that may be treated or inhibited according to the invention include non-solid cancers, e.g. hematopoietic malignancies such as all types of leukemia, e.g. acute lymphocytic leukemia (ALL), acute myelogenous leukemia (AML), chronic lymphocytic leukemia (CLL), chronic myelogenous leukemia (CML), myelodysplastic syndrome (MDS), mast cell leukemia, hairy cell leukemia, Hodgkin's disease, non-Hodgkin's lymphomas, Burkitt's lymphoma and multiple myeloma. Non-limiting examples for solid tumors that may be treated or inhibited according to the invention include tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of Vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma. In some embodiments, the cancer is a solid tumor. In some embodiments, the solid tumor is breast cancer.

MPO in the pathogenesis of multiple inflammatory diseases, including atherosclerosis and cardiovascular disease, kidney disease, pulmonary inflammation, rheumatoid arthritis, skin inflammation, neuronal disease, and metabolic syndrome. More specifically, in glomerular and tubulointerstitial disease, polymorphonuclear- and monocyte-derived reactive oxygen species may contribute to oxidative modification of proteins, lipids, and nucleic acids. In part, the processes instigated by reactive oxygen species parallel events that lead to the development of atherosclerosis. Thus, in some embodiments, the methods and compositions of the invention may be applicable for atherosclerosis, as well as for Cardiovascular diseases for example, Endotoxemia, Myocardial infarction/ischemia, Vascular disfunction and Atrial fibrillation.

Still further, the ability of MPO to generate hypochlorous acid/hypochlorite (HOCl/OCl—) from hydrogen peroxide in the presence of chloride ions is a unique and defining activity for this enzyme. The MPO-hydrogen peroxide-chloride system leads to a variety of chlorinated protein and lipid adducts that in turn may cause dysfunction of cells in different compartments of the kidney. Thus, in some embodiments, the methods and compositions of the invention may be applicable for variety of Kidney diseases, that may include chronic kidney disease, Ischemia/reperfusion injury, Glomerulonephritis and Lupus nephritis.

In yet some further embodiments, the methods, compositions, cells and kits of the invention may be applicable for at least one respiratory condition. Respiratory condition or disease, as used herein encompasses pathological conditions affecting the organs and tissues that are part of the respiratory tract. These conditions include conditions of the respiratory tract including the trachea, bronchi, bronchioles, alveoli, pleurae, pleural cavity, and the nerves and muscles of respiration. Respiratory diseases range from mild and self-limiting, to life-threatening diseases and include chronic and acute conditions. Chronic respiratory diseases (CRDs) are diseases of the airways and other structures of the lung. They are characterized by a high inflammatory cell recruitment (neutrophil) and/or destructive cycle of infection. Respiratory diseases can be classified in many different ways, including by the organ or tissue involved, by the type and pattern of associated signs and symptoms, or by the cause of the disease.

In some embodiments, respiratory conditions encompassed by the invention, include but are not limited to at least one of Pulmonary arterial hypertension (PAH), chronic obstructive pulmonary disease (COPD) and Chronic Granulomatous Disease (GCD). More specifically, Klinke et al. (Klinke et al. (2018) JCI Insight; 3(11): e97530) showed a mechanistic link between MPO and impaired pulmonary vascular function and suggested that pulmonary arterial hypertension may be ameliorated by MPO inhibition. Specifically, they showed that MPO is elevated in patients with pulmonary arterial hypertension and it correlates with subsequent adverse outcomes. In addition they observe that patients with high MPO level (>583 pmol/L) showed decreased survival (P=0.023) over a median of 65 weeks compared with patients with low MPO level. In addition, deficient MPO mice (MPO−/−) showed less increased right ventricular pressure upon hypoxia compared with wild type mice. Hypoxia-induced activation of the Rho-kinase pathway—a critical subcellular signaling pathway involved in vasoconstriction and structural vascular remodeling—was blunted in MPO−/− mice. Thus, this study demonstrates a tight mechanistic link between MPO, the activation of Rho-kinase, and adverse pulmonary vascular function. Therefore, according to some embodiments, the methods of the invention may be applicable for reducing MPO levels in a subject suffering from PAH. In yet some further embodiments, the methods of the invention may be applicable for the treatment of a subject suffering from PAH. Pulmonary arterial hypertension (PAH), as used herein, is a progressive disease without a cure, characterized by remodeling and narrowing of the pulmonary arteries, which lead to elevation of right ventricular pressure, heart failure, and death. The leukocyte-derived heme-enzyme myeloperoxidase (MPO) emerges as a potential mediator of PAH, as it is firmly established as a central mediator of humoral dysfunction of the vessel wall.

Still further, Myeloperoxidase is a strong oxidant stored in primary granules of neutrophils with potent antibacterial and proatherogenic properties. Myeloperoxidase has been implicated in the pathogenesis of chronic obstructive pulmonary disease (COPD). More specifically, increased serum myeloperoxidase levels are associated with rapid lung function decline and poor cardiovascular outcomes in COPD patients, which support the emerging role of myeloperoxidase in the pathogenesis of COPD progression.

Still further, Myeloperoxidase is the major peroxidase enzyme in neutrophil granules and implicated in contributing to inflammatory lung damage in cystic fibrosis. Free myeloperoxidase is present in cystic fibrosis lung fluid and generates hypochlorous acid. Thus, in some embodiments the methods and compositions of the invention may be applicable for variety of Pulmonary inflammatory conditions including, cystic fibrosis, COPD, Sepsis-induced injury, Influenza virus-induced injury, Asbestos-induced injury and Acute lung inflammation.

Other inflammatory diseases applicable in the present invention include Rheumatoid arthritis and Skin inflammation. In offering methods and compositions for modulating the expression, levels, and activity of MPO in a subject in need, the present invention further encompasses the provision of treating disorders associated with reduced or decreased expression and/or activity of MPO, or alternatively or additionally, disorders or conditions that are associated with other factors, but may be ameliorated or affected by modulating the expression and/or activity of MPO. In some embodiments, such conditions or disorders may be referred to herein as associated indirectly with MPO. By modulating and elevating the MPO levels, using the methods of the invention, the present specification further provides methods for treating conditions associated for example with ROS-deficiency and/or MPO-deficiency. Thus, in some embodiments, the MPO-related condition that may be applicable for the methods of the invention may be associated with reduced or deficient ROS levels. A non-limiting example for disorders associated with other factors, for example ROS, may be the Chronic Granulomatous Disease (GCD), that is defined as genetic deficiency in the reactive oxygen species (ROS)-producing phagocyte NADPH oxidase NOX2. In such case, enhancing the levels of MPO, may result in elevation in ROS, thereby providing a tool for treating ROS-associated disorders.

Thus, in some specific embodiments, the invention may be further applicable for Chronic Granulomatous Disease (CGD) that may be associated with ROS disorder. In accordance with some embodiments, the methods of the invention may be applicable for treating MPO deficiency-related condition or any condition caused directly or indirectly by MPO depletion.

It should be appreciated that the invention provides therapeutic methods for treating disorders associated with MPO, for example, AD, MS, P-ANCA-related disorders and PAH, or any of the disorders disclosed by the invention, by eliminating or reducing MPO levels in undifferentiated BM cells of the subject. As disclosed above, the invention provides therapeutic methods compositions, kits and cells that manipulate the immune system of a subject, specifically, by targeting MPO in undifferentiated BM cells of a subject, thereby leading to manipulation of MPO levels in any tissue and organ of said subject. As demonstrated by this aspect, such modulation is effectively used by the invention for therapeutic applications, specifically for treating MPO-related conditions, such as AD, MS, P-ANCA-related disorders and PAH. It should be therefore appreciated that in some specific and non-limiting embodiments, the invention provides methods, compositions and kits for treating subjects that suffer from MPO-related conditions, e.g., AD, MS, P-ANCA-related disorders and PAH, by manipulating the immune-system of the subject, specifically, BM cells of the subject to display modulated levels, expression and/or activity of MPO. More specifically, the therapeutic methods of the invention are based on the provision of a manipulated immune system (either of an autologous or of allogeneic or syngeneic source). Alternatively, the immune system of the subject may be manipulated in vivo, using the gene editing system provided herein (e.g., administering the compounds of the gene editing system to the subject), such that the BM cells population of said subject exhibit modulated levels, expression and/or activity of MPO. As such, the therapeutic methods, kits and compositions of the invention provide effective therapeutic strategy for treating such pathologic conditions. In yet some further specific embodiments, such treatment strategy may be affected by transplanting BM cells that display modulated levels, expression, and/or activity of MPO, to a subject suffering from an MPO-related condition, for example, MS or AD, that in some embodiments was subjected to immune-ablation prior to transplantation. By transplanting BM cells that display modulated (e.g., reduced or eliminated) levels, expression or activity of MPO, the transplanted subjects exhibit modulated expression of MPO in any tissue and organ. It should be understood that as explained in detailed by the invention, the transplanted BM cells may be autologous BM cells that were obtained from the same subject and ex vivo manipulated using the gene editing systems of the invention to modulate the levels, expression or activity of the MPO. For example, in some specific embodiments, BM cells obtained from the subject may be subjected ex vivo to the gene editing systems of the invention. This may result in elimination and reduction of the MPO levels, expression and/or activity. Alternatively, undifferentiated BM cells of an allogeneic or syngeneic source that display (either naturally, or by applying the gene editing systems of the invention) modulated level, expression and/or activity of MPO may be used. Such autologous or allogeneic BM cells that are MPO deficient, may be re-introduced to the subject, that may be in some embodiments, treated with immuno-ablating compounds, and thereby these transplanted manipulated BM cells replace, at least in part, the immune-system of a subject. This procedure results in depletion of MPO in any organ or tissue of the subject. As indicated above, the MPO depleted BM cells may be obtained either from the subject, or alternatively from an allogenic or syngeneic subject. More specifically, transplantation of these autologous or allogeneic BM cells to the subject, or in other words replacement of the immune system cells of the subject with the BM cells that display modulated levels, expression and/or activity of MPO (e.g., eliminated or reduced MPO levels, expression and/or activity), leads to modulation of the levels, expression an activity of MPO in every tissue and organ of said subject, thereby providing a novel and inventive approach for the treatment of any MPO-related disorder or condition, specifically, AD, MS,P-ANCA-related disorders and PAH, or any of the disorders or conditions disclosed by the invention.

As noted above, the invention provides methods for treating disorders as specified above. The term “treatment” as used herein refers to the administering of a therapeutic amount of the composition of the present invention, specifically, either the CRISPR system discussed above, or BM cells that display modulated expression of MPO, which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease from occurring or a combination of two or more of the above. The treatment may be undertaken when an immune-related, autoimmune, neurodegenerative, respiratory, inflammatory or proliferative condition initially develops, or may be a continuous administration, for example by administration more than once per day, every 1 day to 7 days, every 7 day to 15 days, every 15 day to 30 days, every month to two months, every two months to 6 months, or even more, to achieve the above-listed therapeutic effects.

As noted above, the invention further provides a prophylactic tool for preventing MPO associated disorders, based on the expression and/or activity of MPO in a subject, even before the appearance of any symptoms of the disease. The term “prophylaxis” refers to prevention or reduction the risk of occurrence of the biological or medical event, specifically, the occurrence or re-occurrence of disorders associated with an immune-related, autoimmune, neurodegenerative, inflammatory or proliferative conditions, that is sought to be prevented in a tissue, a system, an animal or a human being, by a researcher, veterinarian, medical doctor or other clinician, and the term “prophylactically effective amount” is intended to mean that amount of an active ingredient administered will achieve this goal. Thus, in particular embodiments, the methods of the invention are particularly effective in the prophylaxis, i.e., prevention of conditions associated with any of an immune-related, autoimmune, neurodegenerative, inflammatory or proliferative disorders discussed herein. Thus, subjects treated by the methods of the invention or administered with the compositions are less likely to experience symptoms associated with said neurodegenerative, vascular and/or inflammatory disorders that are also less likely to re-occur in a subject who has already experienced them in the past.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the an immune-related, autoimmune, neurodegenerative, inflammatory or proliferative disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described herein.

The terms “delay” or “delaying the onset” and all variations thereof are intended to encompass the slowing of the progress and/or exacerbation of a disorder associated with an immune-related, autoimmune, neurodegenerative, inflammatory or proliferative disorders and their symptoms slowing their progress, further exacerbation or development, so as to appear later than in the absence of the treatment according to the invention.

As noted above, treatment or prevention include the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction” or “attenuation” as referred to herein, relate to the retardation, restraining or reduction of a process, specifically, any of the an immune-related, autoimmune, neurodegenerative, inflammatory or proliferative disorder by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

The present invention relates to the treatment of subjects, or patients, in need thereof. By “patient” or “subject in need?” it is meant any organism who may be affected by the above-mentioned conditions, and to whom the preventive and prophylactic compositions and methods herein described is desired. More specifically, the composition/s, kit/s and method/s of the invention are intended for preventing pathologic condition in mammals. By “mammalian subject” is meant any mammal for which the proposed therapy is desired, including humans, domestic and non-domestic mammals such as canine and feline subjects, bovine, simian, equine and murine subjects and rodents. It should be noted that specifically in cases of non-human subjects, the method of the invention may be performed using administration via injection, drinking water, feed, spraying, oral lavage and directly into the digestive tract of subjects in need thereof.

It should be appreciated that any systemic or local administration mode may be applicable in the present invention. Routes of administration of the MPO targeting CRISPR system of the invention or any compositions thereof or a BM cell population of the invention or any compositions thereof include, but are not limited to, direct BM injection or transplantations, intracerebral, intraventricular, intracerebroventricular, intrathecal, intracisternal, intraspinal and/or peri-spinal routes of administration by delivery via intracranial or intravertebral needles and/or catheters with or without pump devices. It should be appreciated that in some embodiments any further administration modes may be applicable, for example, intraperitoneal (IP), intravenous (IV) and intradermal, subcutaneous, nasal, pulmonary, oral and intramuscular, administration.

In yet a further aspect, the invention relates to pharmaceutical compositions comprising the gene editing compound, specifically, the at least MPO targeting CRISPR system provided by the invention, in free form and be administered directly to the subject to be treated. Alternatively, in some further embodiments the invention provides pharmaceutical compositions comprising a BM cell population and specifically undifferentiated BM cells that display modulated MPO expression and/or activity, such as HSC, all having modulated expression and/or activity of MPO. Thus, in some embodiments, the pharmaceutical compositions of the invention may comprise a therapeutic effective amount of at least one of: (a) at least one gene editing compound capable of/adapted for modulating the expression and/or activity of MPO in at least one undifferentiated BM cell of a subject in need thereof; and (b) undifferentiated BM cell population exhibiting a modulated expression and/or activity of MPO. It should be noted that the compositions of the invention may optionally further comprise at least one of pharmaceutically acceptable carrier/s, diluent/s and/or excipient/s.

In some embodiments, the pharmaceutical compositions may comprise a therapeutic effective amount of at least one gene editing compound as described herein above, in accordance with other aspects of the invention. More specifically, in some embodiments, the at least one gene editing compound being at least one PEN or any nucleic acid molecule comprising a sequence encoding said PEN or any kit, composition or vehicle comprising the at least one PEN, or any nucleic acid molecule encoding such PEN. In some embodiments, the compositions of the invention may comprise a gene editing compound that may be at least one PEN comprising at least one CRISPR/cas system. In more specific embodiments, the CRISPR/cas system comprised within the composition of the invention may comprise at least one of: (a) at least one CRISPR/cas protein, or any nucleic acid molecule encoding said Cas protein; and (b) at least one nucleic acid sequence comprising at least one gRNA that targets a protospacer within the MPO gene, or any nucleic acid sequence encoding said gRNA. It should be understood that the composition of the invention may comprise any kit, composition or vehicle comprising at least one of (a) and (b).

In some embodiments, the cas protein of the composition of the invention may be a member of at least one of CRISPR-associated system of Class 1 and Class 2. In some specific embodiments, the Cas protein comprised within the composition of the invention may be a member of at least one of CRISPR-associated system of Class 1 and Class 2, specifically, any one of type II, type I, type III, type IV, type V and type VI. In more specific embodiments, such cas protein may be a member of a CRISPR-associated system type II, of class 2. Still further, in some embodiments the pharmaceutical composition of the invention may comprise as a Cas protein, the Cas9 or any fragments, mutants, variants or derivatives thereof.

Thus, in some embodiments, the composition of the invention may comprise the CRISPR/Cas gene editing system. This system may comprise in some embodiments two elements, at least one gRNA, and at least one Cas9, or any mutant, fragment or variant thereof. It should be appreciated that both elements may be provided either as an gRNA and a polypeptide (cas9), or as nucleic acid sequences encoding these elements. In some embodiments, the nucleic acid sequence encoding the gRNA may be provided either alone or in a nucleic acid molecule that comprises also the nucleic acid sequence encoding the Cas9 polypeptide, specifically, in a single nucleic acid molecule or vector. In yet some further embodiments, the composition of the invention may optionally further comprise at least one of pharmaceutically acceptable carrier/s, diluent/s and/or excipient/s. In yet some further embodiments, the composition of the invention may comprise at least one gRNA that targets a protospacer within the MPO gene. More specifically, in some embodiments, the gRNA or crRNA of the invention may target a protospacer comprising the nucleic acid sequence as denoted by SEQ ID NO: 43, SEQ ID NO: 44, SEQ ID NO: 45, SEQ ID NO: 46, as well as SEQ ID NOs. 94, 95, 96, 97, 98, 99, 100, 101, 102, 103, 104, 105, and 106, of the human MPO gene, or any fragments thereof. In yet some particular embodiments, the crRNA (gRNAs) useful in the compositions of the invention may comprise the nucleic acid sequence as denoted by any one of SEQ ID NO: 33, SEQ ID NO: 34, SEQ ID NO: 35, SEQ ID NO: 42, 47, as well as SEQ ID NOS. 50, 55, 60, 63, 66, 71, 74, 77, 80, 85, 88 and 91. In yet some further embodiments, were gRNAs that target protospacers in the mouse MPO gene are used, such gRNAs may comprise the nucleic acid sequence as denoted by any one of SEQ ID NO:5, SEQ ID NO:6, SEQ ID NO:8, SEQ ID NO:9, SEQ ID NO:10, SEQ ID NO:11, SEQ ID NO:12, SEQ ID NO:13 and SEQ ID NO:14. As noted above, in some alternative embodiments, the pharmaceutical composition of the invention may comprise a therapeutic effective amount of BM cell population, specifically undifferentiated BM cell population, exhibiting a modulated expression and/or activity of MPO. The composition of the invention may comprise a kit comprising the cell population or any combination thereof with the gene editing compound discussed above. In some embodiments, the pharmaceutical composition may optionally further comprise at least one of pharmaceutically acceptable carrier/s, diluent/s and/or excipient/s.

As described herein above, the undifferentiated BM cell population that display a modulated expression and/or activity of MPO, may comprise isolated HSC and/or progenitor cells. Still further, in some embodiments, the undifferentiated BM cells of the compositions of the invention may be a BM cell population transduced or transfected with at least one gene editing compound capable of modulating the expression and/or activity of MPO in said cells. It should be understood that in some embodiments, the undifferentiated BM cell population may be either of an autologous source or of an allogenic source. The use of cells of syngeneic source is also encompassed by the invention.

In yet some further embodiments, the undifferentiated BM cell population comprised within the composition of the invention may be a BM cell population of an allogeneic subject exhibiting an inhibited or eliminated expression and/or activity of MPO. It should be understood that any of the cells disclosed herein before in connection with any other aspects of the invention, are also applicable in connection with the present aspect as well.

In certain embodiments, the composition of the invention may be applicable for use in a method for treating, preventing, ameliorating, inhibiting or delaying the onset of an MPO related condition or disease in a mammalian subject as described herein. More specifically, in some embodiments, the composition of the invention may be suitable for use in treating a MPO-related condition that may be at least one of an immune-related disorder, a neurodegenerative disorder, a proliferative disorder, a respiratory disorder, a vascular disorder or any combination thereof. In yet some further specific embodiments, such immune-related disorder may be at least one of an autoimmune disorder and an inflammatory disorder. According to particular embodiments, the invention provides compositions for use in treating autoimmune disorder such as any one of MS, ANCA-related condition, specifically, AAV, AAGN, NCGN, and RPGN, and SLE. In yet some further specific embodiments, the composition of the invention may be applicable for neurodegenerative disorders, in some specific embodiments, neurodegenerative disorders such as AD or PD. In yet some further embodiments, the pharmaceutical composition of the invention may be applicable for treating proliferative disorders, specifically, cancer. Still further embodiments of the invention provides the use of the pharmaceutical composition of the invention for the treatment of inflammatory diseases, specifically, any one of atherosclerosis, RA and IBD. In yet some further embodiments, the invention provides the use of the pharmaceutical composition of the invention for the treatment of a respiratory disease, specifically, for the treatment of PAH. In some alternative embodiments, the invention encompasses the provision of pharmaceutical compositions that lead to an increase in the expression and/or the activity of MPO. In some embodiments the invention further provides composition for use in treating disorders associated with MPO-deficiency, that involve for example, elevation of H₂O₂ levels specifically as discussed herein before. It should be appreciated that any of the disorders or conditions disclosed by the invention in connection with other aspects of the invention, or any stages, symptoms associated therewith, may be also applicable in connection with this aspect as well, specifically, the composition of the invention.

The pharmaceutical compositions of the present invention may be admix in in a pharmaceutically acceptable carrier prior to administration. Therapeutic formulations may be administered in any conventional dosage formulation. Formulations typically comprise at least one active ingredient, as defined above, together with one or more pharmaceutically and physiologically acceptable carriers in the sense of being compatible with the other ingredients and not injurious to the patient. In some specific embodiments, the pharmaceutical composition of the invention may be suitable for injection. The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringeability exists. Nanoscale drug delivery systems using micellar formulations, liposomes and nanoparticles, for example, gold nanoparticles, are emerging technologies for the rational drug delivery, which offers improved pharmacokinetic properties, controlled and sustained release of drugs and, more importantly, lower systemic toxicity. Therefore, in some embodiments, nanoparticles based delivery may be used for the delivery of the CRISPR system of the invention. A particularly desired solution allows for externally triggered release of encapsulated compounds. Externally controlled release can be accomplished if drug delivery vehicles, such as micelles, liposomes or polyelectrolyte multilayer capsules, incorporate nanoparticle (NP) actuators. More specifically, Controlled drug delivery systems (DDS) have several advantages compared to the traditional forms of drugs.

It should be therefore understood that the invention further encompasses the use of various nanostructures, including micellar formulations, liposomes, polymers, dendrimers, metal, specifically, gold, silicon or carbon materials, polymeric nanoparticles and magnetic nanoparticles, as carriers in drug delivery systems. The term “nanostructure” or “nanoparticle” is used herein to denote any microscopic particle smaller than about 100 nm in diameter. In some other embodiments, the carrier is an organized collection of lipids. The pharmaceutical compositions of the invention generally comprise a buffering agent, an agent who adjusts the osmolarity thereof, and optionally, one or more pharmaceutically acceptable carriers, excipients and/or additives as known in the art. As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated. Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

In yet some further embodiments, specifically when in vivo applications are disclosed (e.g., administration of CRISPR/Cas system or any nucleic acid sequence encoding the same, variety of administration modes may be applicable, and therefore, the composition of the invention may be adapted for any of the administrations modes disclosed by the invention, specifically, intraperitoneal (IP), intravenous (IV), parenteral, and intradermal, subcutaneous, topical, nasal, pulmonary, oral, intramedullary, intramuscular, sublingual, buccal, rectal, vaginal, administration. In yet some further embodiments, specifically for respiratory disorders, pulmonary administration is considered a preferred administration mode. The term “administration” when relating to treatment of respiratory disorders is preferably pulmonary delivery by oral inhalation, such as by using liquid nebulizers, aerosol-based metered dose inhalers (MDIs), or dry powder dispersion devices, or by intraperitoneal injection. Alternatively, the administration may be any one of sublingual, buccal, parenteral, intravenous, intramuscular, subcutaneous, intramedullary, or transdermal.

Still further, in another aspect, the invention provides a therapeutically effective amount of at least one of: (a) at least one gene editing compound adapted for modulating the expression and/or activity of MPO in at least one undifferentiated BM cell of said subject; and (b) undifferentiated BM cell population exhibiting a modulated expression and/or activity of MPO, for use in a method for treating, preventing, ameliorating, inhibiting or delaying the onset of an MPO-related condition in a mammalian subject. In some embodiments, the undifferentiated BM cell population is a BM cell population transduced or transfected with at least one gene editing compound capable of modulating the expression and/or activity of MPO in the cells.

In yet some further embodiments, the gene editing compound is at least one PEN comprising at least one CRISPR/cas system, said CRISPR/cas system comprises at least one of: (a) at least one CRISPR/cas protein, or any nucleic acid molecule encoding said Cas protein; and (b) at least one nucleic acid sequence comprising at least one gRNA that targets a protospacer within the MPO gene, or any nucleic acid sequence encoding said gRNA; or any kit, composition or vehicle comprising at least one of (a) and (b).

In yet some further aspect thereof, the invention provides the use of a therapeutic effective amount of at least one gene editing compound capable of modulating the expression and/or activity of MPO in at least one undifferentiated BM cell of said subject, in the preparation of a composition for treating, preventing, ameliorating, inhibiting or delaying the onset of an MPO related condition or disease in a mammalian subject.

In yet some further aspect thereof, the invention provides the use of a therapeutic effective amount of an undifferentiated BM cell population exhibiting a modulated expression and/or activity of MPO in the preparation of a composition for treating, preventing, ameliorating, inhibiting or delaying the onset of an MPO related condition or disease in a mammalian subject. In yet a further aspect, the invention provides a therapeutic kit comprising: at least one of (a) at least one gene editing compound capable of modulating the expression and/or activity of MPO in at least one undifferentiated BM cell of a subject in need; and (b) undifferentiated BM cell population exhibiting a modulated expression and/or activity of MPO.

Still further, in some other aspects, the invention relates to at least one undifferentiated BM cell transduced or transfected with at least one gene editing compound capable of modulating the expression and/or activity of MPO in the cell.

In some specific embodiments, the cell may be of a subject suffering of an MPO-related condition. In yet some further alternative disorder, the cell may be of an allogeneic or syngeneic subject. It should be understood that any of the cells and any of the gene editing compounds disclosed by the invention are also applicable for this aspect as well.

In yet some further embodiments, the invention provides any of the cells disclosed herein for use in a method for modulating the expression and/or activity of MPO in a mammalian subject. In yet some further embodiments, the invention provides any of the cells disclosed herein for use in a method for treating, preventing, ameliorating, inhibiting or delaying the onset of an MPO-related condition in a mammalian subject.

It should be appreciated that any of the MPO-disorders disclosed by the invention are also applicable in the present aspects. Still further, it should be understood that all cells, gene-editing systems disclosed by the invention in connection with any aspects of the invention may be also applicable for any other aspect disclosed by the invention.

As indicated above, MPO is involved in NETosis, thus, by modulating the levels, expression and/or function of MPO, the invention further provides an approach for modulating NETosis. Thus, in yet some further aspects, the invention provides methods for modulating NETosis, specifically in a subject. Still further by providing methods for inhibiting MPO expression and/or activity, the invention further provides in some embodiments thereof methods and compositions for inhibiting, eliminating or reducing NETosis, specifically in a subject in need. In yet some further embodiments, it should be understood that the invention provides methods and compositions for treating and preventing any NETosis associated disorders and conditions in a subject. Upon stimulation, neutrophils rapidly activate the NADPH oxidase to generate superoxide, a highly reactive molecule that dismutates to hydrogen peroxide (H₂O₂). H₂O₂ is consumed by MPO to produce hypochlorous acid (HOCl) and other oxidants. MPO is also required for NET formation, as shown in donors with complete MPO deficiency, but its role remains unclear. Although ROS are cytotoxic, they are also important signaling mediators that regulate protein function via the oxidation of specific amino acid residues. However, since ROS are highly reactive, short-lived molecules, it is unclear how they are able to produce specific cellular responses. In particular, during NET formation, it is not known whether and how ROS regulate the selective translocation of NE from the granules to the nucleus. Furthermore, as the nucleus begins to decondense during NET formation, neutrophil chemotaxis is arrested through an unknown mechanism. Thus, in still a further aspect thereof, the invention provides a method for modulating the properties and function of neutrophil cells.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%.

The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, and/or parts, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law. As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited. Various embodiments and aspects of the present invention as delineated herein above and as claimed in the claims section below find experimental support in the following examples. Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Reagents

MOG35-55 (Blavatnik center, Tel Aviv University)

IFA (Sigma)

Mycobacterium tuberculosis (MTB, H37Ra, Difco)

pertussis toxin (PT, Sigma)

Ketamine (Bremer).

Xylazine (Phibro).

sodium azide (Sigma, USA).

Evans Blue dye (sigma).

Lipofectamine 2000 (Invitrogen)

Restriction Enzymes

BsmBI (NEB).

T4 DNA ligase (NEB)

Plasmids

pL-CRISPR.EFS.GFP (Addgene, catalogue number #57818).

Kits

Magnetic cell isolation kit (Miltenyi Biotech).

DNeasy (Qiagen).

ViraPower Promoterless Lentiviral Gateway Kit (Invitrogen, Carlsbad, Calif., USA).

Lenti-X p24 Rapid Titer Kit (Clontech, Mountain View, Calif., USA).

Antibodies

Mouse anti Myeloperoxidase (abcam); Rabbit anti Neutrophil Elastase (abcam); Rabbit anti Iba1 (abcam); Rat anti GFAP (Invitrogen); Rat anti CD3 (R&D Systems); Mouse anti TAU (abcam);

Mouse anti Amyloid Beta (Covance); Mouse anti Oligodendrocytes (Merck).

Cells

Competent E. Coli cells (JM109, Promega), 293T cells (ATCC, CRL-3216™).

Media and Supplements

Stemspam SFEM medium (Stem cell technologies), TPO, SCF (Peprotech).

Animals

C57BL/6 (Arlan);

Mpo^{Tm1/Lus} (4265, Jackson labs), these mutant mice display impaired fungicidal activity due to myeloperoxidase deficiency. Under hyperlipidemic conditions, mutant mice develop larger atherosclerotic lesions than control mice. These mice are useful for studying systemic inflammatory defenses against pathogens. 5×FAD mice (34840, Jackson labs).

Experimental Procedures

Animal Housing

C57BL/6, Mpo^{Tm1/Lus} and 5×FAD mice were placed in a light-controlled environment (12-h light/dark cycle) and housed in individually ventilated cages (IVC) with free access to food and water. All animal studies were authorized by the Animal Care, Use, and Review Committee of Tel Aviv University (Appendix B—Approval number 01-17-069 and 01-18-010).

Mice Irradiation and Cell Transplantation

Recipient mice were given 9 Gy (3 Gy/minute) whole-body lethal irradiation from a gamma irradiation source (Biobeam GM). 24 hours following irradiation, 2×10⁶ freshly isolated bone marrow cells or 5000 lentivirus-transduced HSCs were intravenously injected into recipient mice through the tail vein using a 27-gauge needle. Mice were monitored and weighed every other day for the following 8 weeks.

Blood Samples MPO Analysis

For validation of MPO knock down, 200 μl blood was taken from the facial vein 8 weeks following transplantation. Blood samples were analyzed for peroxidase activity using the ADVIA2120i (Siemens) hematology system.

Bone Marrow Isolation and HSCs Enrichment

C57BL/6 (Arlan) or Mpo^{Tm1/Lus} (Jackson labs) donor mice were sacrificed and bone marrow was harvested from femurs, tibias and homerus bones using a 27-gauge needle. Erythrocytes were lysed and cells were either transplanted immediately or were subjected to enrichment for Lineage-cells using magnetic cell isolation kit (Miltenyi Biotech). Cells were cultured in Stemspam SFEM medium (Stem cell technologies) supplemented with 50 ng/ml TPO and 100 ng/ml SCF (Peprotech).

EAE Model Induction

14-16 week-old C57BL/6 mice were immunized subcutaneously at three injection sites with 300 μg MOG35-55 (MEVGWYRSPFSRVVHLYRNGK as denoted by SEQ ID NO: 7, Blavatnik center, Tel Aviv University) emulsified in IFA (Sigma) supplemented with 4 mg/ml Mycobacterium tuberculosis (MTB, H37Ra, Difco). On days 0 and 2 post-immunization, 300 ng of pertussis toxin (PT, Sigma) was administered intraperitoneally in 200 μl of phosphate buffered saline (PBS). Disease severity was monitored daily and EAE clinical disease scored according to the following scale: 0, no paralysis; 0.5, partial loss of tail tonicity; 1, complete loss of tail tonicity; 2, hind limb weakness and partial hind limb paralysis; 3, complete hind limb paralysis; 4, hind limb paralysis and forelimb weakness or partial forelimb paralysis 5, moribund state; 6, death.

Behavioral Examinations

Behavioral examinations were executed starting from seven months of age. Two weeks prior to the beginning of examinations, the mice were acclimated to a reversed light environment. Before each trial, the mice were transferred to the behavioral testing room 30 minutes prior to the beginning of the trial habituation. The order of the trials was counterbalanced across four treatments and genotypes. Mazes were cleaned with Virusolve+ between trials. Open field, elevated plus maze, Y maze and Morris water maze tests, were recorded, monitored and analyzed by an automated tracking system (Ethovision, Noldus).

Open Field was performed to assess risk assessment/anxiety-related behavior. The test was conducted in a white, perspex, empty arena (50×50×40 cm). The mice were placed in the center of the arena and could explore it for 15 minutes. Total distance traveled, as well as the time spent in the center of the maze and the corners of the maze were measured.

Elevated plus maze was performed to assess risk assessment/anxiety-related behavior. The test was conducted in an apparatus consisting of four cross-shaped white perspex arms (35×5 cm) and was lifted 40 cm from the floor. Two arms, facing each other, were enclosed by 15 cm high walls, while the other two arms were open. The mice were placed in the center of the maze facing a closed arm and was recorded for 7 minutes. The time spent in either the open or the closed arms of the maze was measured. Mice that fell off the open arms were excluded from analysis.

Y-Maze: Two-trial Y-maze was performed to assess spatial memory. The test was conducted in a white, perspex Y-shape apparatus with arm length of 38 cm, width of 5 cm and height of 15 cm. The test consisted of a sample trial and a test trial. In the sample trial, the mice were placed at the end of one arm of the maze facing the wall, while one arm of the maze was blocked, and could explore the two arms of the maze for 5 minutes. The sample trial was followed by a 5 minutes inter-trial interval. In the test trial, the mice were returned to the maze with all arms open for another 5 minutes. Novel arm entry (Number of novel arm entries/total arm entries) and novel arm exploration time (novel time exploration time/total arms exploration time) were measured for the first minute of the test trial.

Morris water maze was performed to assess spatial learning. The test was conducted in a pool (150 cm diameter, 30 cm deep) filled with water made opaque with skim milk Black rectangle, triangle and circle cues were placed on the pool walls. The water temperature was 27±1° C. A transparent platform (12 cm×12 cm) was placed at the center of one quadrant of the pool, about 2 cm below the water surface. Each mouse undertook four trials per day for 5 consecutive days, starting each day from a different quadrant of the three quadrants not containing the platform. In each trial, a mouse was placed into the water and allowed to find the hidden platform for 60 sec. If the mouse failed to find the platform within 60 sec., it was guided to the platform and allowed to stay there for 10 sec. The latency to find the hidden platform within 60 sec., as well as total distance covered were recorded for each mouse. Mice that failed to find the platform were scored as have reached the platform in 60 sec.

Fear Conditioning test was performed to assess associative memory. The test was performed in a fear conditioning chamber (Ugo Basile, 17×17×25 cm). On day 1, the mice were placed in the chamber for 5 minutes for habituation. On day 2, the mice were placed again in the chamber for 3.5 minutes. After 2 minutes, the mice received two foot-shocks (0.8 mA, 2 s) with a one-minute interval. On day 3, the mice were placed in the chamber for 3 minutes. Fear conditioning was evaluated by scoring freezing behavior—the absence of all movement except for respiration.

Histology

Mice are injected with a mixture of Ketamine/Xylazine (100/ and 10 mg/kg, respectively) intraperitoneally. Then, using an electric pump, mice are intracardially perfused with PBS followed by ice-cold 4% paraformaldehyde (PFA) in PBS. The brains are removed and post-fixed in 4% PFA at 4° C. for 24 hours and then cryopreserved in 30% sucrose. Subsequently, brains are stored in PBS with 0.02% sodium azide (Sigma, USA) at 4° C. until immunohistochemical processing. Sucrose treated brains are dried then snap frozen in 2-Methylbutane in liquid Nitrogen. Brains are sectioned (10 μm) using a cryostat and mounted directly onto slides for analysis. Slices are blocked and permabilized with PBS supplemented with 10% normal goat serum, 1% bovine serum albumin and 0.5% Triton-100 for 1 hour and are incubated overnight with primary antibodies followed by 1 hour incubation with secondary antibodies.

Immunostaining

Brains were snap frozen in 2-methylbutane cooled in liquid nitrogen and embedded in OCT before cutting. Coronal sections (10 μm) were cut using a freezing sliding microtome (Leica CM1850) and stored at −20° C. until use. For S100β immunostaining, slides were washed two times with PBS, blocked and permeabilized with PBS 1% bovine serum albumin, 5% goat serum (Biological Industries, Israel) and 0.05% Triton-X (Sigma-Aldrich) for one hour and incubated with S100β antibody (1:500, ab66028, Abcam) overnight in 4° C. Slides were washed 3 times with PBS and incubated with secondary goat anti mouse antibody (1/700, Alexa-Flour) for 1 hour at room temperature. DNA was stained with DAPI (1:1000, Sigma-Aldrich). For Thioflavin S (ThioS) staining, slides were incubated for 8 minutes with 0.01% ThioS solution in 50% ethanol. Slices were then briefly incubated twice for ten seconds with 80% ethanol, and washed twice with double distilled water (DDW).

Confocal Imaging

Fluorescence images were obtained using a confocal microscope LSM710 (Carl Zeiss Micro Imaging). The confocal images were captured using a 10× and a 20× objectives (NA=0.3, 0.8 respectively, Plan-Apochromat). Fluorescence emissions resulting from Ar 488 and 543 nm laser lines for EGFP and CY3 respectively, were detected using filter sets supplied by the manufacturer. For DAPI detection we used our mode-locked Ti:Sapphire, femtosecond pulsed, multiphoton laser (Chameleon Ultra II, Coherent, Inc.) at a wavelength of 720 nm. Images were generated using the Zeiss ZEN 2011 software (Carl Zeiss, Inc.). All images were exported in TIF and their contrast and brightness were optimized using ImageJ. S100β mean intensity and ThioS plaque density were analyzed using ImageJ.

Tissue Collection for RNA and Protein Analyses

For RNA and protein analyses, the mice were anesthetized with ketamine/xylazine and were immediately perfused with PBS. The brains were removed, and hippocampi were dissected, divided into left and right hemispheres, snap-frozen in liquid nitrogen and stored at −80° C. until use. For bone marrow RNA analysis, femur bones were collected, and bone marrow was extracted by flushing with a 27G needle into PBS. Bone marrow cells were frozen in Tri-reagent (Sigma) and stored at −80° C. until use.

Protein Extraction and Western Blot

Hippocampi were thawed and homogenized in lysis buffer (200 mM HEPES, 5 mM EDTA, 1% Nonidet P-40, 0.5% sodium deoxycholate, 1 mM Na2VO4, 150 mM NaCl and 50 mM NaF) supplemented with protease inhibitor (Roche). Cells were incubated for one hour at 4° C. Proteins were cleared by centrifugation at 14,000×g for 20 min at 4° C. Protein concentrations were quantified utilizing the Pierce™ BCA Protein Assay Kit (Thermo Scientific). 25 μg protein from each sample was resolved in SDS-PAGE. Nitrocellulose transferred membranes were blocked for one hour with PBS 0.1% Tween 20 with 5% bovine serum albumin and were probed with goat anti APOE antibody (1:10,000, Chemicon) overnight in 4° C. and with goat anti ACTIN antibody (1:5000, MAB1501, Milipore) for one hour at room temperature, followed by incubation with Goat anti Mouse secondary antibody (1:5,000, Licor) for one hour at room temperature. Visualization and analysis of band intensities were performed using the Odyssey system (Licor) and the Image Studio Lite 5.2 software. For each sample, APOE results were normalized to ACTIN.

RNA Extraction and Real Time PCR

Hippocampal RNA was extracted using RNeasy Mini Kit (Qiagen). RNA from bone marrow samples was extracted as previously described and was cleaned using RNeasy Mini Kit (Qiagen). RNA was reverse transcribed to complementary DNA (cDNA) using verso cDNA synthesis kit (Thermo-Scientific). Semi-quantitative PCR was performed on the Step-One Real time PCR system using Syber-Green Master mix (Thermo-Scientific) and the custom designed primers. Threshold cycle values were determined in triplicates and presented as average compared with Actin. Fold changes were calculated using the 2^{-ΔΔCT} method.

Primer Set List (all for Mouse Genes)

APOE
(SEQ ID NO: 17)
Forward, 5′-GAGCTGATCTGTCACCTCCG-3′
and
(SEQ ID NO: 18)
Reverse 5′-GACTTGTTTCGGAAGGAGC-3′;
CCL2
(SEQ ID NO: 19)
Forward, 5′-GGGATCATCTTGCTGGTGAA-3′
and
(SEQ ID NO: 20)
Reverse 5′-AGGTCCCTGTCATGCTTCTG-3′;
CXCL10
(SEQ ID NO: 21)
Forward, 5′-AACTGCATCCATATCGATGAC-3′
and
(SEQ ID NO: 22)
Reverse 5′-GTGGCAATGATCTCAACAC-3′;
IL1β
(SEQ ID NO: 23)
Forward, 5′-GGAGAACCAAGCAACGACAAAATA-3′
and
(SEQ ID NO: 24)
Reverse 5′-TGGGGAACTCTGCAGACTCAAAC-3′;
MPO
(SEQ ID NO: 25)
Forward, 5′-TGGTGGCCTGCAGAGTATGA-3′
and
(SEQ ID NO: 26)
Reverse 5′-GTTGAGGCCAGTGAAGAAGG-3′;
S100β
(SEQ ID NO: 27)
Forward, 5′-TTGCCCTCATTGATGTCTTCCA-3′
and
(SEQ ID NO: 28)
Reverse 5′-TCTGCCTTGATTCTTACAGGTGAC-3′;
TNFα
(SEQ ID NO: 29)
Forward, 5′-AGGGTCTGGGCCATAGAACT-3′
and
(SEQ ID NO: 30)
Reverse 5′-CCACCACGCTCTTCTGTCTAC-3′;
VCAM1
(SEQ ID NO: 31)
Forward, 5′-GGAGCCTGTCAGTTTTGAGAATG-3′
and
(SEQ ID NO: 32)
Reverse 5′-TTGGGGAAAGAGTAGATGTCCAC-3′.

Statistical Analysis

All data are expressed as the mean±SEM. Statistical analysis was performed using GraphPad Prism 7. Behavioral data as well as bone marrow MPO mRNA levels were analyzed using unpaired student's t-test. Other mRNA analyses, blotting quantification, S100β intensity quantification and ThioS plaque density were analyzed using Mann-Whitney nonparametric test. For all tests, the statistical significance threshold was set to p<0.05.

BBB Permeability Assay

A 2% solution of Evans Blue dye in saline (4 ml/kg of body weight) is injected intra-peritoneally to mice. One hour after injection, mice are perfused with PBS, and brains and spinal cords are taken and homogenized. Supernatant from homogenate is measured for absorbance at 610, indicating evans blue penetration.

MPO CRISPR/Cas9 Plasmid Cloning

The mouse MPO targeting guide RNA (gRNA) gRNA-3 (CCCCAACGATCAGCTGACCA), as denoted by SEQ ID NO: 5, and gRNA-4 (CAGCGGGGTGTACGGCAGCG), as denoted by SEQ ID NO: 6, as well as gRNA2 (GCACTCATGTTCATGCAGTG) as denoted by SEQ ID NO: 8 and gRNA 6 (TGCGATACTTGTCATTCGGT) as denoted by SEQ ID NO: 9 were designed using the Azimuth 2.033 tool. The pL-CRISPR.EFS.GFP plasmid (gift from Benjamin Ebert, addgene plasmid #57818) was digested with BsmBI (NEB) and gRNA sequences were ligated using T4 DNA ligase (NEB). Ligated plasmids were transformed into competent E. Coli cells (JM109, Promega), selected using ampicillin and insert ligation was validated by Sanger sequencing. MPO targeting was assessed by transfection into 293T cells followed by DNA extraction (DNeasy, Qiagen) and Sanger sequencing.

Lentiviral Particles Production

Ligated plasmids were cloned using the ViraPower Promoterless Lentiviral Gateway Kit (Invitrogen, Carlsbad, Calif., USA). Plasmids (3 μg) were co-transfected with the packaging plasmids (9 μg): pLP1, pLP2, and pLP/VSVG using Lipofectamine 2000 (Invitrogen) into the 293 T producer cell line. The lentiviral titer was determined using the Lenti-X p24 Rapid Titer Kit and the manufacturer's recommended procedure (Clontech, Mountain View, Calif., USA).

Lentiviral Transduction of Isolated HSCs

24 hour following isolation, HSCs were transduced with MPO CRISPR/Cas9 lentivirus in the presence of 4 μg/ml polybrene and 1 μg/ml rapamycin. Transduced cells were either transplanted immediately into lethally irradiated mice or grown ex vivo for CRISPR/Cas9 activity analysis. GFP expression was evident after 1 day in culture.

### Example 1

Targeting MPO in the EAE Model of Multiple Sclerosis

EAE, or Experimental Allergic Encephalomyelitis, is an induced inflammatory demyelinating disease of the central nervous system (CNS) which is widely accepted as an animal model of human CNS demyelinating diseases, including, but not limited to, multiple sclerosis (MS) and acute disseminated encephalomyelitis (ADEM). EAE can be induced in a number of species, including mice, rats, guinea pigs, rabbits and primates. Disease induction is usually done by exposure of the animals to various antigens. The most commonly used antigens are spinal cord homogenate (SCH), purified myelin, myelin protein such as myelin basic protein (MBP), Myelin proteolipid protein (PLP or lipophilin), and Myelin Oligodendrocyte Glycoprotein (MOG), or peptides of these proteins, all resulting in distinct models with different disease characteristics regarding both immunology and pathology.

Depending on the antigen used and the genetic make-up of the animal, rodents can display a monophasic bout of EAE, a relapsing-remitting form, or chronic EAE. The typical susceptible rodent will debut with clinical symptoms around two weeks after immunization and will present symptoms of a relapsing-remitting disease. Modeling of multiple sclerosis may be performed using C57BL/6 female mice, in which disease is induced with myelin-oligodendrocyte glycoprotein peptide (MOG). This model represents progressive (also referred to as chronic) form of the disease. The scoring index indicated in the experimental procedures is used for monitoring the severity of the disease and the onset of relapses in order to determine the therapeutic effect of the compositions and kits of the invention that modulate and specifically reduce or inhibit MPO expression and/o activity.

Establishment of the EAE Model

The EAE model of MS was induced by immunization of adult female mice with an adjuvant containing the MOG35-55 peptide, together with 2 immunizations with Pertussis Toxin (PT). In order to determine the minimal amount of these substances required to establish EAE model, C57BL/6 female mice were immunized with either 200 μg MOG35-55 and 200 ng PT, 200 μg MOG35-55 and 300 ng PT or 300 μg MOG35-55 and 300 ng PT. While no difference was shown in mice clinical score between the first two doses, the higher dose of 300 μg MOG35-55 and 300 ng PT evoked a severe disease in all immunized mice (FIG. 1). This result indicates that this dose is the most adequate to enhance an EAE disease.

BM Transplantation Rescue Lethality in Irradiated C57BL/6 Mice

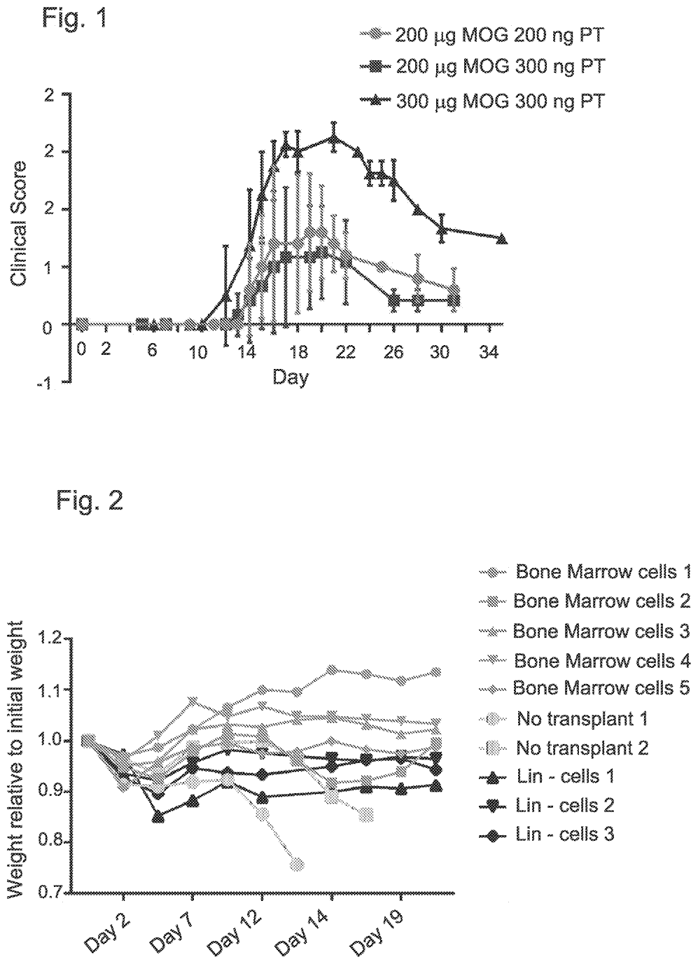

In order to determine irradiation dose lethality and BM cells transplantation's hematopoietic reconstitution ability, seven 8-week old male C57BL/6 mice were irradiated (9Gy) and five of them were transplanted with one million freshly isolated BM through the tail vein. Expectedly, all mice displayed 4-10% decline in weight after 48 hours (FIG. 2). While all transplanted mice showed a sharp recovery and return to their initial weight in 2 weeks, non-transplanted mice deteriorated and deceased after 13-15 days. These results indicate that the 9 Gy dose used is lethal. Moreover, all 5 mice transplanted with BM cells were able to recover after 14 days, indicating that indeed a complete hematopoietic reconstitution was established upon transplantation.

In order to establish a more homogenous cell population for CRISPR-Cas9 mediated KO experiments, BM cells were further enriched for hematopoietic progenitors. This was performed by enriching BM for cells that does not express markers of mature hematopoietic cells (i.e. Lin-cells) using magnetic activated cell sorting (MACS). In order to validate that these cells retain hematopoietic reconstitution potential, these cells were also transplanted into lethally irradiated C57BL/6 mice Similar to BM transplanted mice, these mice also showed a sharp decline in weight after 48 hours followed by a recovery and survival (FIG. 2). This result implies that Lin-hematopoietic progenitors retain their hematopoietic reconstitution potential and are adequate for CRISPR-Cas9 KO experiments.

Transplantation of BM from MPO^{Tm1/Lus} into EAE Mice

In order to assess the effect of myeloid-MPO deficiency in EAE mice model of MS, twenty eight female C57BL/6 mice were lethally irradiated as described above and BM cells (1×106 in 200 μl) derived from either the WT C57BL/6 mice that normally express MPO, or the MPO knock-out Mpo^{Tm1/Lus} mice, were transplanted into the irradiated EAE mice. Table 1 Summarizes the experimental groups used in this experiment.

TABLE 1
Grouping of Mpo^{Tm1/Lus}-derived bone marrow cells EAE experiment

| | | N. of | | Transplanted cells | EAE |
|---|---|---|---|---|---|
| | Strain | mice | Irradiation | origin | induction |
| A | C57BL/6 | 14 | V | C57BL/6 | V |
| B | C57BL/6 | 14 | V | Mpo^{Tm1/Lus} | V |
| C | C57BL/6 | 14 | X | No transplantation | V |
| D | Mpo^{Tm1/Lus} | 4 | X | No transplantation | V |

The influence of MPO deficiency on EAE disease course is assessed by measuring disease clinical score, as described in experimental procedures. Mice of all experimental groups are also evaluated by post mortem analyses that examine EAE-related aspects such as neuronal loss, synaptic loss and microgliosis as well as BBB permeability, leukocyte infiltration into the CNS, microgliosis and NETosis rate.

### Example 2

Targeting MPO in the FAD Model of Alzheimer Disease (AD)

Transplantation of BM from Mpo^{Tm1/Lus} into 5×FAD Mice—Examining Peroxidase Activity

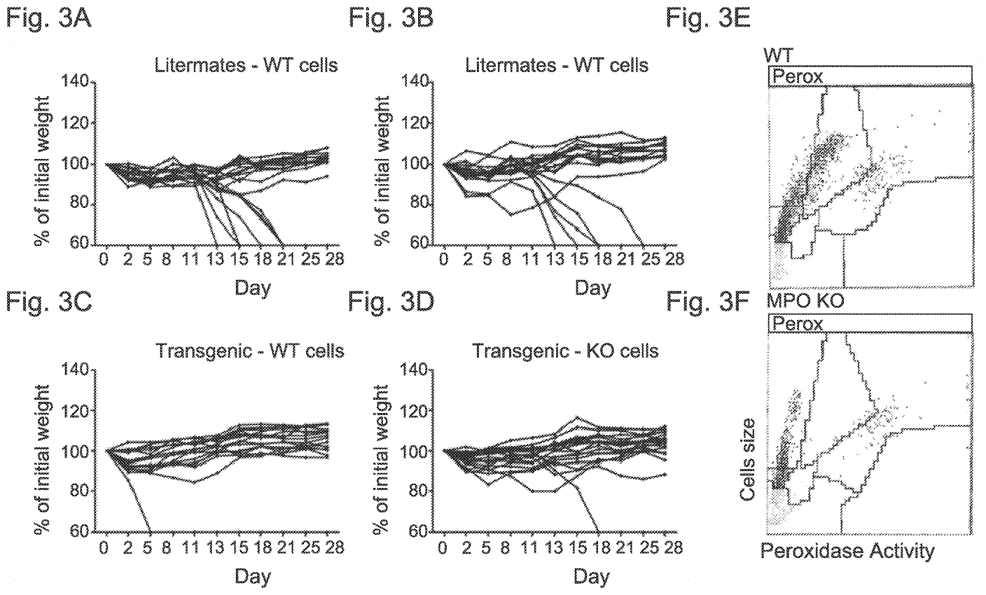

In order to assess the effect of myeloid-MPO deficiency in 5×FAD mice model of AD, 70 male 5×FAD were lethally irradiated as well as their non-transgenic littermates and transplanted BM cells (1×10⁶ in 200 μl) derived from either WT C57BL/6 mice that normally express MPO, or the MPO knock-out (KO) Mpo^{Tm1/Lus} mice, were transplanted into the irradiated AD mice. The experimental groups are summarized in Table 2. Four weeks following transplantation, 84% of mice survived and return to their initial weight (FIG. 3A-3D). Eight weeks following transplantation, peroxidase activity was measured in blood samples from mice subjected to either WT or MPO KO BM cell transplantation. Indeed, blood samples from mice subjected to transplantation of BM cells obtained from MPO KO mice showed less peroxidase activity (FIG. 3E-3F), confirming MPO KO hematopoietic reconstitution of MPO KO cells in these mice.

TABLE 2
Grouping of MpoTm1/Lus-derived bone marrow cells 5 × FAD experiment

| | Strain | N. of mice | Irradiation | Transplanted cells origin |
|---|---|---|---|---|
| A | 5 × FAD | 17 | V | Mpo^{Tm1/Lus} |
| B | 5 × FAD | 16 | V | C57BL/6 |
| C | Littermates | 13 | V | Mpo^{Tm1/Lus} |
| D | Littermates | 13 | V | C57BL/6 |
| E | 5 × FAD | 7 | X | No transplantation |
| F | Littermates | 7 | X | No transplantation |

Transplantation of BM from Mpo^{Tm1/Lus} into 5×FAD Mice—Examining MPO-mRNA Levels

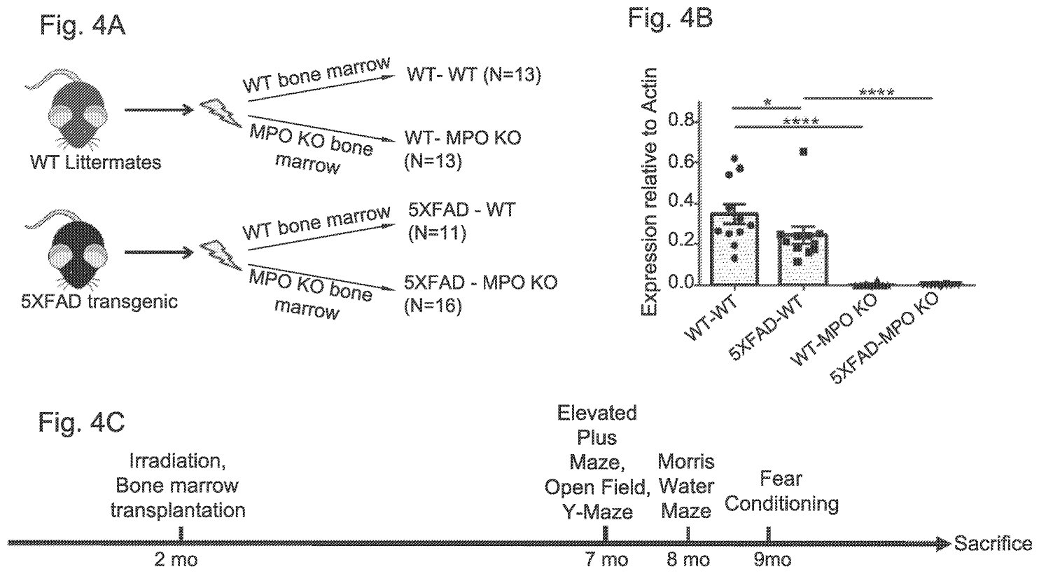

Hematologic MPO deficiency in adult 5×FAD mice, and their non-transgenic littermates were produced. Two-month old mice were subjected to hematopoietic ablation by exposure to myeloablative irradiation, followed by re-population with bone marrow (BM) cells derived from either MPO-KO or WT C57BL/6 mice. This resulted in four experimental groups; (1) WT with WT BM (WT-WT), (2) WT with MPO-KO BM (WT-MPO KO), (3) 5×FAD mice with WT BM (5XAD-WT) and (4) 5XAD with MPO-KO BM (5×FAD-MPO KO) (FIG. 4A). To confirm the MPO reduction in mice injected with MPO-KO BM, MPO-mRNA expression analysis was performed. Almost undetectable MPO-mRNA levels were found in BM derived from WT-MPO KO and 5×FAD-MPO KO mice, as compared to high levels in the controls indicating for efficient exchange of the hematopoietic population in the transplanted mice (FIG. 4B).

MPO Deficiency Inhibit Cognitive Impairment in 5×FAD Mice

The effect of MPO KO on cognitive impairment in 5×FAD mice was examined. To this aim, the transplanted mice were subjected to a series of behavioral examinations, starting from 7 months of age (FIG. 4C). In the open field test, 5×FAD mice are known to show impaired risk assessment reflected by spending more time at the center of the maze, rather than the periphery. Indeed, 5×FAD-WT mice spent significantly less time at the corners of the maze and more time at the center of the maze, compared to WT-WT mice. In contrast, 5×FAD-MPO KO mice spent significantly less time at the center of the maze compared to 5×FAD-WT mice and exhibited a comparable behavior to as the control WT mice groups (FIG. 5A-5B). Notably, no difference was measured between the four experimental groups in the total distance traveled, indicating for similar motor capacity (FIG. 5C).

In the elevated plus maze, WT mice tend to spend more time in the closed arms of the maze, while 5×FAD mice show tendency towards the open, exposed arms, reflecting impaired risk/anxiety-related assessment. In this experiment, both 5×FAD-WT and 5×FAD-MPO KO mice spent significantly more time in the open arms, reflecting perturbed risk assessment behavior. However, 5×FAD-MPO KO mice spent significantly less time in the open arms of the maze compared to 5×FAD-WT mice (FIG. 5D-5E). Together, these results indicate that MPO KO reduces the risk assessment/anxiety-related behavior in 5×FAD mice.

Next, it was tested whether MPO deficiency would inhibit the cognitive decline seen in 5×FAD mice. In the Y-maze test, 5×FAD-WT mice showed reduced exploration time and less entries to the novel arms as compared to WT-WT, while 5×FAD-MPO KO mice demonstrated about 50% more entries to the novel arms of the maze as compared to 5×FAD WT (FIG. 6A). No significant difference in exploration time was measured between 5×FAD-WT and 5×FAD-MPO KO mice (FIG. 6B). Spatial learning capacity was then examined in these mice in the Morris water maze. During the five days of the trial, 5×FAD-WT mice showed significantly longer latency to find the platform from day 3 of the trial, reflecting impaired spatial learning (FIG. 6C). However, 5×FAD-MPO KO mice showed marked better learning capacity, demonstrated by significantly less latency to find the platform at day 5, as compared to 5×FAD-WT mice (FIG. 6C). The learning improvement in WT mice could also be reflected in the total distance traveled by the mice, as in early days these mice traveled a longer distance than in later days. While 5×FAD-WT mice exhibited relatively consistent traveling distance throughout the trial, 5×FAD-MPO KO mice showed shorter traveling distance as the test progressed, similar to WT groups (FIG. 6D). Finally, associative learning was assessed by using the fear conditioning test. In this test, freezing behavior was measured one day following conditioning with aversive foot shocks. While WT-WT and WT-MPO KO mice showed ˜40% freezing behavior, reflecting proper associative memory reconsolidation, 5×FAD-WT mice showed significantly less freezing behavior. Notably, MPO deficient mice show higher freezing behavior in 5×FAD mice by 2.45-fold (FIG. 6E). Thus, it was demonstrated in five different behavioral tests, that hematologic MPO deficiency inhibits cognitive function impairment in the 5×FAD model of AD.

MPO KO Mice Groups Show Similar Amyloid-β Plaque Load

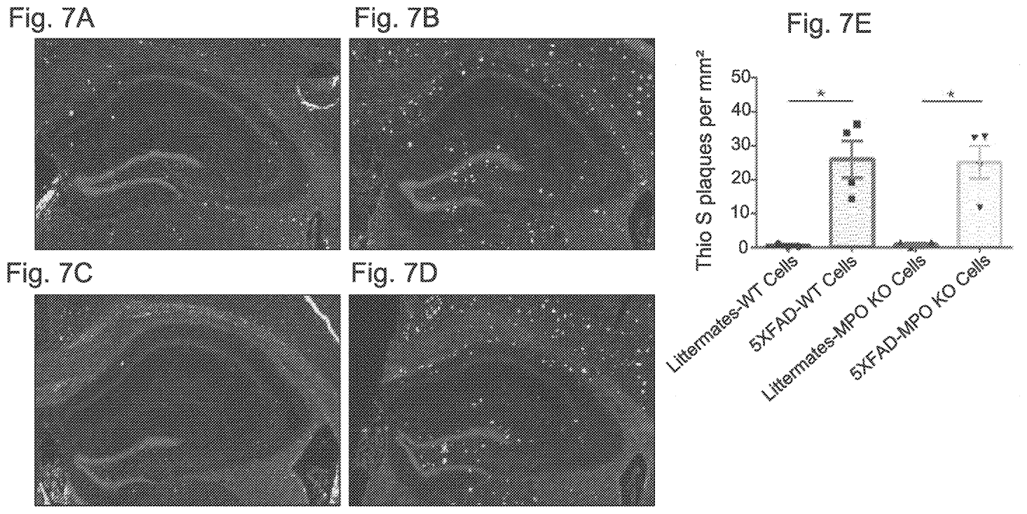

Following the behavioral examinations, the mice were sacrificed and ThioS staining was performed on brain sections to determine the impact of MPO KO on plaque density. While WT mice groups showed no detectable ThioS staining, both 5×FAD-WT and 5×FAD-MPO KO mice groups showed comparable high levels of amyloid-β plaques, indicating that MPO KO had no effect on the generation and accumulation of amyloid-β in the brain of 5×FAD mice (FIG. 7A-7E).

5×FAD-MPO KO Mice Show Reduced Inflammation

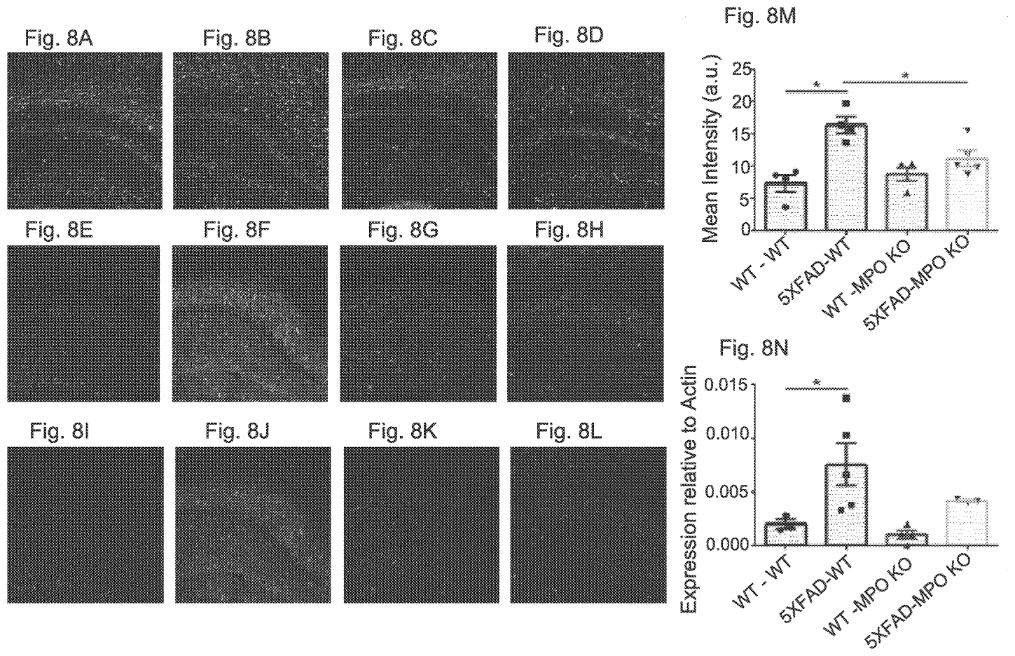

To evaluate the effect of MPO deficiency on brain inflammation, hippocampal sections were stained with antibody against S100B. This protein is known to be overexpressed in activated astrocytes and has been implicated in dystrophic neurites formation in AD. Expectedly, 5×FAD-WT mice exhibited significantly high S100β expression in their hippocampus when compared to WT-WT mice (FIG. 8A-8M). However, 5×FAD-MPO KO mice exhibited low S100β levels comparable to 5×FAD-WT mice, indicating a more restricted inflammatory response in these mice. Real-time PCR (RT-PCR) analysis of hippocampal mRNA samples also showed a significant upregulation in S100β expression in 5×FAD-WT mice when compared to WT-WT mice, mirrored by a limited expression pattern in MPO deficient 5×FAD mice (FIG. 8N).

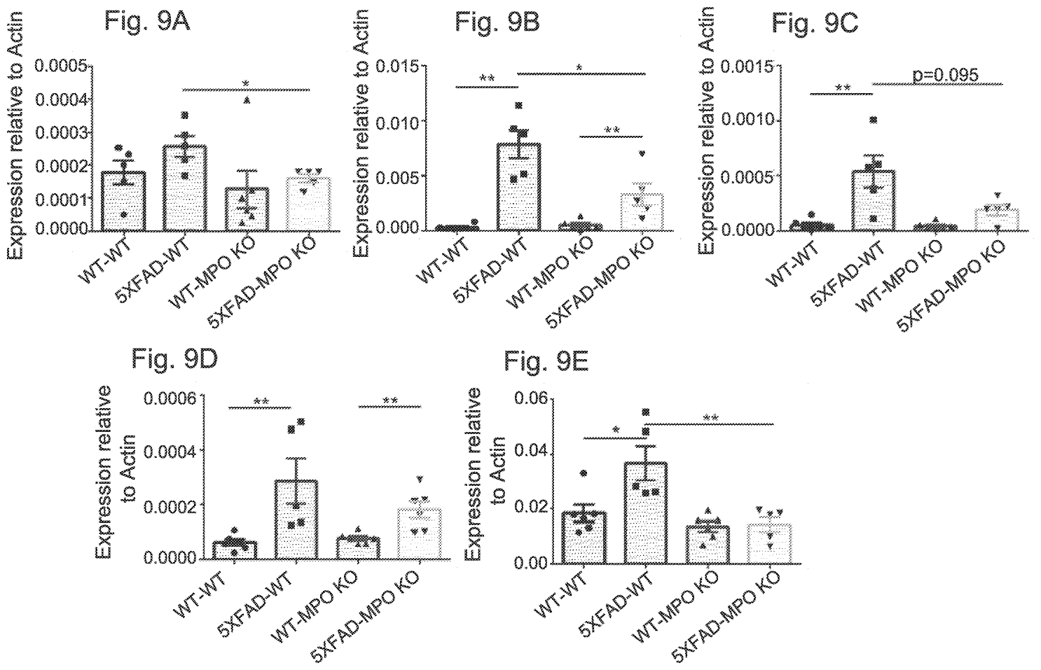

To further examine the effect of MPO deficiency on inflammation, RT-PCR was used to analyze hippocampal mRNA expression of several recognized inflammatory markers. MPO deficient 5×FAD mice showed significantly diminished expression levels of the pro-inflammatory mediators 1L1β and CXCL10 (FIGS. 9A-9B) and to some extent reduced CCL2 and TNFα expression (FIG. 9C-9D). Interestingly, VCAM1 levels that are enhanced in 5×FAD-WT mice were diminished in 5×FAD-MPO KO mice (FIG. 9E). Given the known association of VCAM1 with endothelial impairment, this result may suggest a beneficial effect of MPO KO on endothelium integrity in 5×FAD mice.

MPO Deficiency Reduces APOE Expression in 5×FAD Mice.

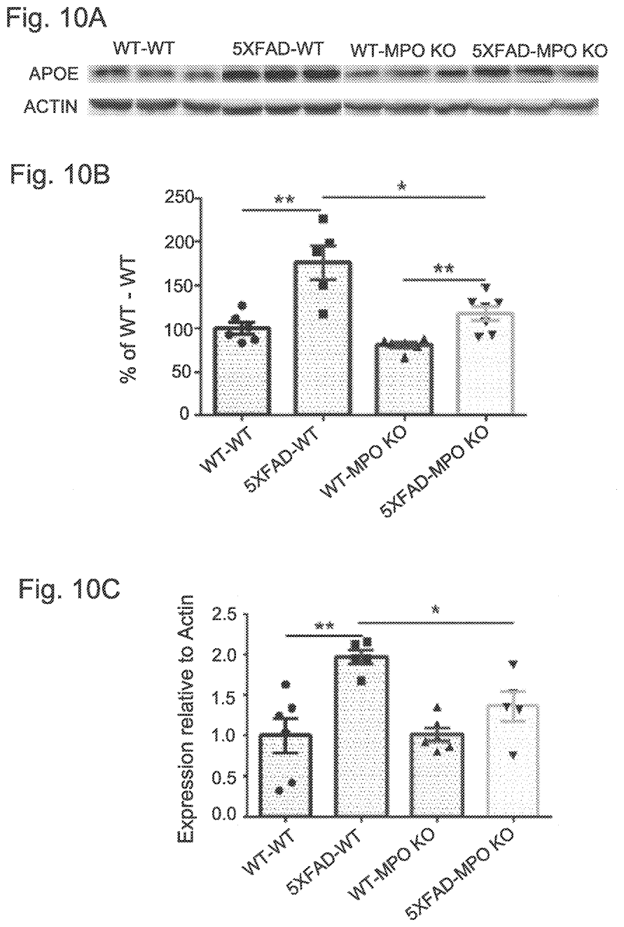

The ε4 allele of the APOE genotype is considered as the largest genetic risk factor for sporadic late-onset AD. While the exact role of APOE in AD is still under debate, APOE clearly plays a central role in AD progression and has been linked to many pathological processes relevant to AD, including amyloid metabolism and aggregation and tau pathology. Interestingly, APOE expression was found to substantially increase in the brains of 5×FAD mice. Indeed, western blot analysis of hippocampal protein lysates showed a significant increase in the levels of APOE in the brains of 5×FAD-WT mice when compared to WT-WT mice, while APOE levels were significantly lower in 5×FAD-MPO KO mice (FIG. 10A, FIG. 10B). This result was further validated on the RNA level (FIG. 10C), indicating that MPO deficiency prevented APOE elevation within the CNS.

### Example 3

MPO Knock-Out Using CRISPR/Cas9 Technology in Isolated HSCs

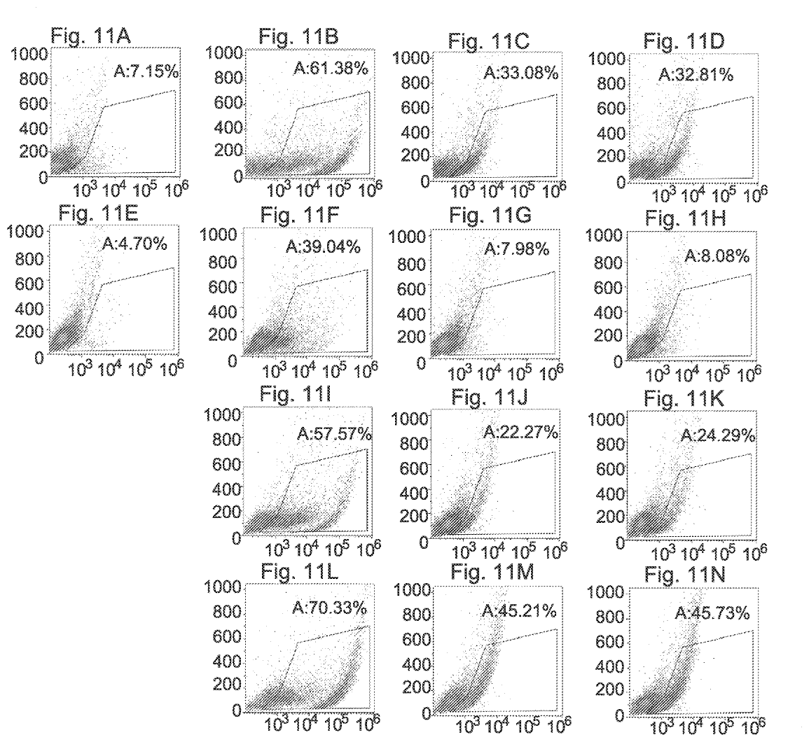

CRISPR/Cas9-based gene alteration is considered as an efficient platform for gene-based therapy. Isolation and ex-vivo culture of target cells facilitate the utilization of this approach to hematopoietic lineage-related diseases. The inventors therefore utilize this platform for lentiviral-mediated MPO knock out in isolated HSCs. For this purpose, bone marrow cells are isolated and enriched for HSCs using magnetic-based cell sorting. These cells are transduced with a lentiviral vectors expressing the Cas9 protein, GFP and guide RNAs (gRNAs) targeting MPO. Following 24 hours in culture, GFP positive cells are FACS-sorted and injected into lethally irradiated model animals. For the generation of lentiviral vectors targeting MPO, various gRNA primers were designed and cloned into the pL-CRISPR.EFS.GFP plasmid. Proper ligation was validated using Sanger sequencing. Next, these plasmids were used for the generation of Lentiviral particles using Virapower lentiviral packaging kit (Invitrogen). Proper lentiviral assembly and integration into hematopoietic stem cells (HSCs) was assessed by transducing isolated HSCs at ascending concentrations (0.5, 2 and 10 μl per 200,00 cells). As control, a GFP expressing Lentivirus was used. GFP analysis 24 and 48 hours following transduction confirmed that HSCs were indeed transduced (FIG. 11), exhibiting 33% and 45% GFP+ cells under the highest concentration examined, following 24 and 48 hours in culture, respectively. These results indicate that isolated HSCs can be transduced and FACS-selected after 24 hours for transplantation into irradiated mice.

GFP positive cells are FACS-sorted and injected into lethally irradiated model animals Subsequent animal experiment with lentiviral-transduced HSCs are performed both in the EAE model and 5×FAD model. Lentivirus vectors expressing no gRNAs are used as controls for these experiments. Grouping for these experiments is described in Table 3 (for EAE experiment) and Table 4 (for 5×FAD experiment).

TABLE 3
Grouping of lentiviral-transduced HSCs EAE experiment

| | | | | EAE |
|---|---|---|---|---|
| | Strain | Irradiation | Transplanted cells origin | induction |
| A | C57BL/6 | V | MPO-KO transduced HSCs | V |
| B | C57BL/6 | V | No gRNA transduced HSCs | V |
| C | C57BL/6 | V | MPO-KO transduced HSCs | X |
| D | C57BL/6 | V | No gRNA transduced HSCs | X |
| E | Mpo^{Tm1/Lus} | X | No transplantation | V |
| F | Mpo^{Tm1/Lus} | X | No transplantation | X |

TABLE 4
Grouping of lentiviral-transduced HSCs 5 × FAD experiment

| | Strain | Irradiation | Transplanted cells origin |
|---|---|---|---|
| A | 5 × FAD | V | MPO-KO transduced HSCs |
| B | 5 × FAD | V | No gRNA transduced HSCs |
| C | Littermates | V | MPO-KO transduced HSCs |
| D | Littermates | V | No gRNA transduced HSCs |
| E | 5 × FAD | X | No transplantation |
| F | Littermates | X | No transplantation |

### Example 4

MPO Knock-Out Using CRISPR/Cas9 Technology in Human Cells (HEK293)

Human HEK293 cells were transfected with Ribonucleoproteins (RNP) complexes containing Cas9 protein, fluorescently labeled tracrRNA and each of the following crRNA sequences that target the human MPO encoding sequences (SEQ ID NO. 33-35 and 42) and the corresponding protospacers (SEQ ID Nos. 43-46, respectively); (PAM appearing in bold):

T173: TGCACATCCCGGTGATGGTG,
as denoted by SEQ ID NO: 33;
D260: CAGGGGTGAAGTCGAGGTCG,
as denoted by SEQ ID NO: 34;
H502: GGATGAGGGTGTGGCCGTAG,
as denoted by SEQ ID NO: 35;
and
C319: GGATGGTGATGTTGCTCCCG,
as denoted by SEQ ID NO: 42.
T173: TGCACATCCCGGTGATGGTGCGG,
as denoted by SEQ ID NO: 43;
D260: CAGGGGTGAAGTCGAGGTCGTGG,
as denoted by SEQ ID NO: 44;
H502: GGATGAGGGTGTGGCCGTAGCGG,
as denoted by SEQ ID NO: 45;
and
C319: GGATGGTGATGTTGCTCCCGGGG,
as denoted by SEQ ID NO: 46.

As indicated by the following Example 5, these particular targets, may be also used in addition to targets for knockout of the MPO gene, also as HDR targets leading to amino acid substitutions that form conformational changes in the protein or changes in protomer biosynthesis and processing (C319A), substitutions that mimic human MPO deficiency substitutions (T173C), amino acid substitutions that interfere with protomer heme binding D260A and H502A. Transfection was made using the Alt-R system, according to the Manufacturer's protocol (Integrated DNA Technologies, Inc., USA). 72 hours following transfection, cells were either harvested for DNA extraction, or FACS sorted for enrichment of cells labeled with the tracrRNA, cultured for another 72 hours, and then harvested for DNA extraction. DNA samples were analyzed using the T7 endonuclease I (T7EI) mismatch detection assay, according to the Manufacturer's protocol (Integrated DNA Technologies, Inc., USA). More specifically, the T7EI assay was used to evaluate the gene editing efficiency of CRISPR-Cas9 reagents at a given guide RNA target site in a population of edited cells.

The following primers were used for the T7EI assay:

1. For T173 gRNA:

Fw Primer (~182 bps downstream):
T173_T7_Fw: TCTTCCTCCAGGGAGTCTCA
as denoted by SEQ ID NO: 36.
Rv Primer (~673 bps downstream):
T173_T7_Rv: GCAGGAAGGAGACAGGTCAT
as denoted by SEQ ID NO: 37.

Amplicon Size: 915

2. For D260 gRNA:

Fw Primer (~621 bps downstream): D260_T7_Fw:
TCTTCCTCCAGGGAGTCTCA as denoted by SEQ ID NO: 36.
Rv Primer (~234 bps downstream): D260_T7_Rv:
GCAGGAAGGAGACAGGTCAT as denoted by SEQ ID NO: 37.

Amplicon Size: 915

3. For C319 gRNA:

Fw Primer (~263 bps downstream): C319_T7_Fw:
CCAGGTTCCCAGTTCAGTGT as denoted by SEQ ID NO: 38.
Rv Primer (~558 bps downstream): C319_T7_Rv:
CCACAGCGTTCTCTGTCCTT as denoted by SEQ ID NO: 39.

Amplicon Size: 881

4. For H502 gRNA:

Fw Primer (~297 bps downstream): H502_T7_Fw:
ATGCCTCTCAGTGCCACTCT as denoted by SEQ ID NO: 40.
Rv Primer (~566 bps downstream): H502_T7_Rv:
AGCCAGGTCAGGGGAAGTAT as denoted by SEQ ID NO: 41.

Amplicon Size: 923.

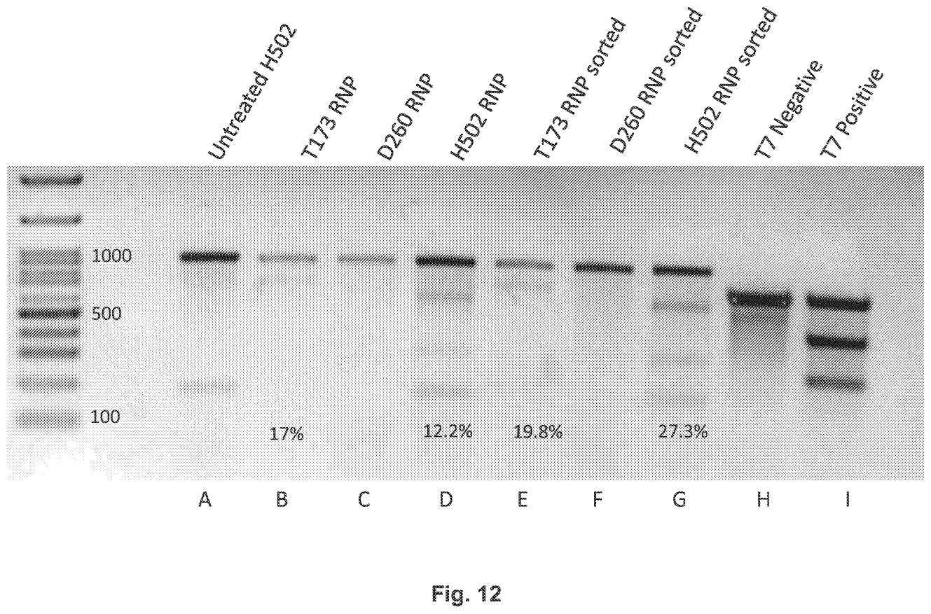

As shown in FIG. 12, cleavage of MPO was observed in human cells, particularly, as shown in sorted cells transfected with H502 (27.3%), and the T173 (19.8%) crRNAs. These results clearly demonstrate the feasibility of specific and effective targeting of MPO in human cells.

### Example 5

Using the CRISPR/Cas9 System for Manipulating Human MPO Function and Levels

Encouraged by the successful knockout of MPO in human cells using the gene editing systems of the invention, the inventors next use the CRISPR/Cas9 system as a tool to substitute specific amino acid residues in the human MPO that elicit changes in MPO biosynthesis and function. The specific target amino acid resides are subdivided into the following groups:

1. Amino acid substitutions that form conformational changes in the protein or changes in protomer biosynthesis and processing—C167A, C180A, C319A, C158A, R128A, N355A.

2. Amino acid substitutions that mimic human MPO deficiency substitutions—T173C, M251T, R569W, R499C, G501S.

3. Amino acid substitutions that interfere with protomer heme binding—Q257A, D260A, M409A, E408A, H261A, H502A.

4. Amino acid substitution that interfere with proper protein glycosylation—N355A. The target residues specified herein relate to the human MPO amino acid sequence as denoted by SEQ ID NO. 2.

Cysteine167

Cysteine167 is a conservative amino acid in the light chain of MPO that forms a disulfide bridge with C180. Substitution of this amino acid residue with a neutral amino acid, prevents the formation of the disulfide bridge.

Cysteine to Alanine substitution, C167A (Change TGC to GCC/GCA/GCT/GCG) is performed as described herein.

A Cas9-mediated Double-strand break (DSB) within the codon sequence of Cysteine167, is performed using the following gRNA sequence:

C167A-1: GGACGTGGGGGTGACTT\hat{ }GCC, as denoted by SEQ ID NO. 47. This gRNA targets the protospacer that comprises the nucleic acid sequence:

GGACGTGGGGGTGACTT\hat{ }GCCCGG, as denoted by SEQ ID NO.
94.
Before cut: CAGGACGTGGGGGTGACTTGC¹⁶⁷CCGGAGCAGGACAA
A, as denoted by SEQ ID NO. 48.
After cut: CAGGACGTGGGGGTGACTT\hat{ }GC¹⁶⁷CCGGAGCAGGACAA
A, as denoted by SEQ ID NO. 48.

(PAM sequence is underlined, \hat{ } marks cut site, Bold letters mark Codon that is substituted). A single stranded oligodeoxynucleotide (ssODN) is used as a template for HDR. ssODNs contain two homology arms flanking the new codon sequence. Homology arms are symmetric, with 50 bases from each side of the PAM.

Additionally, a PAM blocking mutation is introduced to the PAM sequence, in order to prevent CRISPR re-cutting.

The template ssODN oligo for C167A, comprises:

GTTGTCCAAGTCAAGCGGCTGCGCCTACCAGGACGTGGGGGTGACT
GCC¹⁶⁷CCTGAGCAGGACAAATACCGCACCATCACCGGGATGTGCAA
CAACAGGTGCGGC, as denoted by SEQ ID NO. 49.

Cysteine180

Cysteine 180 is a conservative amino acid in the light chain of MPO that forms a disulfide bridge with C167. Substitution of this amino acid residue with a neutral amino acid, prevents the formation of the disulfide bridge.

Cysteine to Alanine substitution C180A (Change TGC to GCC/GCA/GCT/GCG), is performed by Cas9-mediated DSB within the codon sequence of Cysteine180, using the following gRNA sequence:

C180A-1: TCACCGGGATGTGCAAC\hat{ }AAC, as denoted by SEQ ID NO. 50. [symbol] This gRNA targets the protospacer that comprises the nucleic acid sequence

TCACCGGGATGTGCAAC\hat{ }AACAGG, as denoted by SEQ ID NO.
95.
Before cut: ACCATCACCGGGATGTGC¹⁸⁰AACAACAGGTGCGGCTG
GCTG, as denoted by SEQ ID NO. 51.
After cut: ACCATCACCGGGATGTGC¹⁸⁰AAC\hat{ }AACAGGTGCGGCTG
GCTG, as denoted by SEQ ID NO. 51.

(PAM sequence is underlined, \hat{ } marks cut site, Bold letters mark Codon that is substituted). A single stranded oligodeoxynucleotide (ssODN) is used as a template for HDR. ssODNs contain two homology arms flanking the new codon sequence. Homology arms are symmetric, with 50 bases from each side of the PAM.

Additionally, a PAM blocking mutation is introduced to the PAM sequence, in order to prevent CRISPR re-cutting.

The template ssODN oligo for C180A:

CTTGCCCGGAGCAGGACAAATACCGCACCATCACCGGGATGGCC¹⁸⁰A
ACAACAGCTGCGGCTGGCTGGGGGTGGCTGCAGGAACCGGGCTCAGAG
AGGCGTCCCG, as denoted by SEQ ID NO. 52.

Tyrosine173

A missense mutation in the codon of Tyrosine 173, resulting in a replacement to Cysteine was previously reported to result in MPO deficiency, causing abnormal protein processing leading to its degradation.

Thus, as shown by Example 4, a Tyrosine to Cysteine substitution Y173C (Change TAC to TGT/TGC) was performed by a Cas9-mediated DSB within the codon sequence of Tyrosine173, using the following gRNA sequence:

Y173C-1: TGCACATCCCGGTGATG\hat{ }GTG, as denoted by SEQ ID NO. 33, targeting the protospacer that comprises the nucleic acid sequence: TGCACATCCCGGTGATG\hat{ }GTGCGG as denoted by SEQ ID NO. 43.

Before cut:
TGCCCGGAGCAGGACAAATAC¹⁷³CGCACCATCACCGGG,
as denoted by SEQ ID NO. 53.
After cut:
TGCCCGGAGCAGGACAAATAC¹⁷³CGCAC\hat{ }CATCACCGGG,
as denoted by SEQ ID NO. 53.

(PAM sequence is underlined, \hat{ } marks cut site, Bold letters mark Codon that is substituted). A single stranded oligodeoxynucleotide (ssODN) was used as a template for HDR. ssODNs contain two homology arms flanking the new codon sequence. Homology arms are symmetric, with 50 bases from each side of the PAM.

Additionally, a PAM blocking mutation was introduced to the PAM sequence, in order to prevent CRISPR re-cutting.

The template ssODN oligo for Y173C:

GGCTGCGCCTACCAGGACGTGGGGGTGACTTGCCCGGAGCAGGACAAA
TGT¹⁷³CGCACCATCACCGGGATGTGCAACAACAGGTGCGGCTGGCTGG
GGGTGGCTG, as denoted by SEQ ID NO. 54.

Glutamine 257

Glutamine 257 forms and hydrogen bond with the heme cavity and was reported to be involved in halide binding. Substitution of this amino acid with a neutral amino acid, prevents the interaction of its carbonyl residues to water molecules and the formation of hydrogen bonds. Glutamine to Alanine substitution, Q257A (Change CAG to GCC/GCA/GCT/GCG) is performed by Cas9-mediated DSB within the codon sequence of Glutamine 257, using the following gRNA sequence:

Q257A-1: CATGCAATGGGGCCAGC\hat{ }TGT, as denoted by SEQ ID NO. 55. This gRNA targets the protospacer that comprises the nucleic acid sequence

CATGCAATGGGGCCAGC\hat{ }TGTTGG, as denoted by SEQ ID NO.
96.
Before cut: CATGTTCATGCAATGGGGCCAG²⁵⁷CTGTTGGACCACG
A, as denoted by SEQ ID NO. 56.
After cut: CATGTTCATGCAATGGGGCCAG²⁵⁷C\hat{ }TGTTGGACCACG
A, as denoted by SEQ ID NO. 56.

(PAM sequence is underlined, \hat{ } marks cut site, Bold letters mark Codon that is substituted). A single stranded oligodeoxynucleotide (ssODN) is used as a template for HDR. ssODNs contain two homology arms flanking the new codon sequence. Homology arms are symmetric, with 50 bases from each side of the PAM.

Additionally, a PAM blocking mutation is introduced to the PAM sequence, in order to prevent CRISPR re-cutting.

The template ssODN oligo for Q257A:

GACTCCGGACCAGGAGCGCTCACTCATGTTCATGCAATGGGGC
GCG²⁵⁷CTGTTTGACCACGACCTCGACTTCACCCCTGAGCCGGC
CGCCCGGGCCTCCTTCGTC, as denoted by SEQ ID NO. 57.

Aspartate260

Aspartate 260 forms an ester bond with the pyrrole group of the heme, presumably the only covalent bond of MPO light chain to the heme group. Substitution of this amino acid residue with a neutral amino acid, prevents this protein-heme interaction.

Aspartate to Alanine substitution, D260A (Change GAC to GCC/GCA/GCT/GCG) was performed as disclosed by Example 4, using Cas9-mediated DSB within the codon sequence of Aspartate260, using the following gRNA sequence:

D260A-1: CAGGGGTGAAGTCGAGG\hat{ }TCG, as denoted by SEQ ID NO. 34, that targets a protospacer comprising the nucleic acid sequence CAGGGGTGAAGTCGAGG\hat{ }TCGTGG, as denoted by SEQ ID NO. 44.

Before cut:
GCAATGGGGCCAGCTGTTGGAC²⁶⁰CACGACCTCGACTTCACCCCT,
as denoted by SEQ ID NO. 58.
After cut:
GCAATGGGGCCAGCTGTTGGAC²⁶⁰CACGA\hat{ }ACCTCGACTTCACCCCT,
as denoted by SEQ ID NO. 58.

(PAM sequence is underlined, \hat{ } marks cut site, Bold letters mark Codon that is substituted). A single stranded oligodeoxynucleotide (ssODN) was used as a template for HDR. ssODNs contain two homology arms flanking the new codon sequence. Homology arms are symmetric, with 50 bases from each side of the PAM.

Additionally, a PAM blocking mutation was introduced to the PAM sequence, in order to prevent CRISPR re-cutting.

The template ssODN oligo for Q260A:

CCGGACCAGGAGCGCTCACTCATGTTCATGCAATGGGGCCAGCTGTTG
GCA²⁶⁰CACGACCTCGACTTCACCCCTGAGCCGGCCGCCCGGGCCTCCT
TCGTCACTG, as denoted by SEQ ID NO. 59.

Methionine409

Methionine 409 forms an ester bond with the pyrrole group of the heme, presumably the only covalent bond of MPO light chain to the heme group. Substitution of this amino acid residue with a neutral amino acid, prevents this protein-heme interaction.

A Methionine to Alanine substitution, M409A (Change ATG to GCC/GCA/GCT/GCG) is performed by Cas9-mediated DSB within the codon sequence of Methionine 409, using the following gRNA sequence:

M409A-1: GGTGAGCTCGGGCATCT\hat{ }CAC, as denoted by SEQ ID NO. 60. This gRNA targets the protospacer that comprises the nucleic acid sequence:

GGTGAGCTCGGGCATCT\hat{ }CACTGG, as denoted by SEQ ID NO.
97.
Before cut: AGGGGACACCCGTTCCAGTGAGATG⁴⁰⁹CCCGAGCTCA
CCTCCA, as denoted by SEQ ID NO. 61.
After cut: AGGGGACACCCGTTCCAGTG\hat{ }AGATG⁴⁰⁹CCCGAGCTCA
CCTCCA, as denoted by SEQ ID NO. 61.

(PAM sequence is underlined, \hat{ } marks cut site, Bold letters mark Codon that is substituted). A single stranded oligodeoxynucleotide (ssODN) is used as a template for HDR. ssODNs contain two homology arms flanking the new codon sequence. Homology arms are symmetric, with 50 bases from each side of the PAM.

Additionally, a PAM blocking mutation is introduced to the PAM sequence, in order to prevent CRISPR re-cutting.

The template ssODN oligo for M409A:

GAGGCCTTAAGAATGACAGTGGCTTTTGCCTCCCCAAGGGGACACCCGTT
ACAGTGAGGCG⁴⁰⁹CCCGAGCTCACCTCCATGCACACCCTCTTACTTCGG
GAGCAC, as denoted by SEQ ID NO. 62.

Glutamate408

Glutamate 408 forms an ester bond with the pyrrole group of the heme. Substitution of this amino acid residue with a neutral amino acid, prevents this protein-heme interaction.

A Glutamate to Alanine substitution, e408a, (Change GAG to GCC/GCA/GCT/GCG) is performed by Cas9-mediated DSB within the codon sequence of Glutamate 408, using the following gRNA sequence:

E408A-1: GGTGAGCTCGGGCATCT\hat{ }CAC, as denoted by SEQ ID NO. 63.

This gRNA targets the protospacer that comprises the nucleic acid sequence:

GGTGAGCTCGGGCATCT\hat{ }CACTGG, as denoted by SEQ ID NO.
98.
Before cut: AGGGGACACCCGTTCCAGTGAG⁴⁰⁸ATGCCCGAGCTCA
CCTCCA, as denoted by SEQ ID NO. 64.
After cut: AGGGGACACCCGTTCCAGTG\hat{ }AG⁴⁰⁸ATGCCCGAGCTCA
CCTCCA, as denoted by SEQ ID NO. 64.

(PAM sequence is underlined, \hat{ } marks cut site, Bold letters mark Codon that is substituted). A single stranded oligodeoxynucleotide (ssODN) is used as a template for HDR. ssODNs contain two homology arms flanking the new codon sequence. Homology arms are symmetric, with 50 bases from each side of the PAM.

Additionally, a PAM blocking mutation is introduced to the PAM sequence, in order to prevent CRISPR re-cutting.

The template ssODN oligo for E408A:

GAGGCCTTAAGAATGACAGTGGCTTTTGCCTCCCCAAGGGGACACCCGTT
ACAGTGCG⁴⁰⁸ATGCCCGAGCTCACCTCCATGCACACCCTCTTACTTCGG
GAGCAC, as denoted by SEQ ID NO. 65.

Histidine261

Histidine 261 is the distal ligand that coordinates the heme iron. Substitution of this amino acid residue with a neutral amino acid, prevents this protein-heme interaction.

A Histidine to Alanine substitution, H261A (Change CAC to GCC/GCA/GCT/GCG) is performed by Cas9-mediated DSB within the codon sequence of Histidine 261 using the following gRNA sequence:

H261A-1: CAGGGGTGAAGTCGAGG\hat{ }TCG, as denoted by SEQ ID NO. 66. This gRNA targets the protospacer that comprises the nucleic acid sequence:

CAGGGGTGAAGTCGAGG\hat{ }TCGTGG CAGGGGTGAAGTCGAGG\hat{ }TCGTGG,
as denoted by SEQ ID NO. 99.
Before cut: GCAGCGCAGCAGGGACCAC²⁶¹GACCTCCCAGGTGAGG
GGCT, as denoted by SEQ ID NO. 67.
After cut: GCAGCGCAGCAGGGACCAC²⁶¹GAACCTCCCAGGTGAGG
GGCT, as denoted by SEQ ID NO. 67.

(PAM sequence is underlined, \hat{ } marks cut site, Bold letters mark Codon that is substituted). A single stranded oligodeoxynucleotide (ssODN) is used as a template for HDR. ssODNs contain two homology arms flanking the new codon sequence. Homology arms are symmetric, with 50 bases from each side of the PAM.

Additionally, a PAM blocking mutation is introduced to the PAM sequence, in order to prevent CRISPR re-cutting.

The template ssODN oligo for H261A:

ATGAGGATTGGGCTGGACCTGCCTGCTCTGAACATGCAGCGCAGCAGGGA
CGCC²⁶¹GACCTCCCAGGTGAGGGGCTGCAGGAGTCTCCCCTGATGCTCA
CCTCCCC, as denoted by SEQ ID NO. 68.

Cysteine 319

Cysteine 319 forms the only disulfide bridge between the two protomers comprising MPO homodimer. Substitution mutation of Cysteine 319 to Alanine interferes with MPO biosynthesis, and diminishes its peroxidase activity.

A Cysteine to Alanine substitution, C319A (Change TGC to GCC/GCA/GCT/GCG), was performed as shown by Example 4, using Cas9-mediated DSB within the codon sequence of Cysteine 319 using the following gRNA sequence:

C319A-1: GGATGGTGATGTTGCTC\hat{ }CCG, as denoted by SEQ ID NO. 42, that targets the protospacer comprising the nucleic acid sequence

GGATGGTGATGTTGCTC\hat{ }CCGGGG, as denoted by SEQ ID NO.
46.
Before cut: CCTGGCCCAGATCTCATTGC³¹⁹CCCGGATCATCTGCG
ACAA, as denoted by SEQ ID NO. 69.
After cut: CCTGGCCCAGATCTCATTGC³¹⁹CCCGGAATCATCTGCG
ACAA, as denoted by SEQ ID NO. 69.

(PAM sequence is underlined, \hat{ } marks cut site, Bold letters mark Codon that is substituted). A single stranded oligodeoxynucleotide (ssODN) was used as a template for HDR. ssODNs contain two homology arms flanking the new codon sequence. Homology arms are symmetric, with 50 bases from each side of the PAM.

Additionally, a PAM blocking mutation was introduced to the PAM sequence, in order to prevent CRISPR re-cutting.

The template ssODN oligo for C319A:

GGGTGTGTTCAGCATGCAGCAGCGACAGGCCCTGGCCCAGATCTCATGC
C³¹⁹ACCGGATCATCTGCGACAACACAGGCATCACCACCGTGTCTAAGAA
CAACAT, as denoted by SEQ ID NO. 70.

Methionine 251

Missense mutation in the coding region of Methionine 251, resulting in a substitution to Threonine causes inherited MPO deficiency. In Vitro data suggest that M251T substitution affects MPO processing and reduce peroxidase activity.

A Methionine to Threonine substitution, M251A (Change ATG to ACC/ACA/ACT/ACG), is performed using Cas9-mediated DSB within the codon sequence of Methionine 251 using the following gRNA sequence:

M251A-1: GCTCACTCATGTTCATG\hat{ }CAA, as denoted by SEQ ID NO. 71. This gRNA targets the protospacer that comprises the nucleic acid sequence:

GCTCACTCATGTTCATG\hat{ }CAATGG, as denoted by SEQ ID NO.
100.
Before cut: GAGCGCTCACTCATG²⁵¹TTCATGCAATGGGGCCAGCT
GTTG, as denoted by SEQ ID NO. 72.
After cut: GAGCGCTCACTCATG²⁵¹TTCATG\hat{ }CAATGGGGCCAGCT
GTTG, as denoted by SEQ ID NO. 72.

(PAM sequence is underlined, \hat{ } marks cut site, Bold letters mark Codon that is substituted). A single stranded oligodeoxynucleotide (ssODN) is used as a template for HDR. ssODNs contain two homology arms flanking the new codon sequence. Homology arms are symmetric, with 50 bases from each side of the PAM.

Additionally, a PAM blocking mutation is introduced to the PAM sequence, in order to prevent CRISPR re-cutting.

The template ssODN oligo for M251A:

CCACTGATCAGCTGACTCCGGACCAGGAGCGCTCACTCACG²⁵¹TTCATG
CAATGTGGCCAGCTGTTGGACCACGACCTCGACTTCACCCCTGAGCCGGC
CGCCCG, as denoted by SEQ ID NO. 73.

Arginine 569

Missense mutation in the coding region of Arginine 569, resulting in a substitution to Tryptophan causes inherited MPO deficiency. In Vitro data suggest that R569W substitution affect MPO processing, prevents heme incorporation and diminishes peroxidase activity.

A Arginine to Tryptophan substitution, R569W (Change CGG to TGG), is performed using Cas9-mediated DSB within the codon sequence of Arginine 569 using the following gRNA sequence:

R569W-1: AATTGCAGTGGATGAGA\hat{ }TCCGGG as denoted by SEQ ID NO. 74. This gRNA targets the protospacer that comprises the nucleic acid sequence:

AATTGCAGTGGATGAGA\hat{ }TCCGGG as denoted by SEQ ID NO.
101.
Before cut: GCAGTGGATGAGATCCGG⁵⁶⁹GAGCGATTGTTTGAGCA
GG, as denoted by SEQ ID NO. 75.
After cut: GCAGTGGATGAGA\hat{ }TCCGG⁵⁶⁹GAGCGATTGTTTGAGCA
GG, as denoted by SEQ ID NO. 75.

(PAM sequence is underlined, \hat{ } marks cut site, Bold letters mark Codon that is substituted). A single stranded oligodeoxynucleotide (ssODN) is used as a template for HDR. ssODNs contain two homology arms flanking the new codon sequence. Homology arms are symmetric, with 50 bases from each side of the PAM.

Additionally, a PAM blocking mutation is introduced to the PAM sequence, in order to prevent CRISPR re-cutting.

The template ssODN oligo for R569W:

CACCCCTGCCAAGCTGAATCGTCAGAACCAAATTGCAGTGGATGAGATCT
GA⁵⁶⁹GAGCGATTGTTTGAGCAGGTCATGAGGATTGGGCTGGACCTGCCT
GCTCTG, as denoted by SEQ ID NO. 76.

Arginine499

Inherited substitution mutation of Arginine to Cysteine in position 499 results in MPO deficiency. Arginine 499 was shown to be important for the stabilization of the critical Histidine 502.

An Arginine to Cysteine substitution, R499C (Change CGC to TGT/TGC), is performed using Cas9-mediated DSB within the codon sequence of Arginine 499, using the following gRNA sequence:

R499C-1: TTCACCAATGCCTTCCG\hat{ }CTA, as denoted by SEQ ID NO. 77. This gRNA targets the protospacer that comprises the nucleic acid sequence:

TTCACCAATGCCTTCCG\hat{ }CTACGG as denoted by SEQ ID NO.
102.
Before cut: ACCAATGCCTTCCGC⁴⁹⁹TACGGCCACACCCTCATC,
as denoted by SEQ ID NO. 78.
After cut: ACCAATGCCTTCCG\hat{ }C⁴⁹⁹TACGGCCACACCCTCATC,
as denoted by SEQ ID NO. 78.

(PAM sequence is underlined, \hat{ } marks cut site, Bold letters mark Codon that is substituted).

A single stranded oligodeoxynucleotide (ssODN) is used as a template for HDR. ssODNs contain two homology arms flanking the new codon sequence. Homology arms are symmetric, with 50 bases from each side of the PAM.

Additionally, a PAM blocking mutation is introduced to the PAM sequence, in order to prevent CRISPR re-cutting.

The template ssODN oligo for R499C:

GACTCAGTGGACCCACGCATCGCCAACGTCTTCACCAATGCCTTCTG
C⁴⁹⁹TACCGCCACACCCTCATCCAACCCTTCATGTTCCGCCTGGACAATC
GGTACCAGC, as denoted by SEQ ID NO. 79.

Glycine501

Inherited substitution mutation of Glycine to Serine in position 501 results in MPO deficiency. Glycine 501 was shown to be important for the stabilization of the critical Histidine 502.

A Glycine to Serine substitution, G501S (Change GGC to AGT/AGC), is performed by Cas9-mediated DSB within the codon sequence of Glycine 501, using the following gRNA sequence: G501S-1: TTCACCAATGCCTTCCG\hat{ }CTA as denoted by SEQ ID NO. 80. This gRNA targets the protospacer that comprises the nucleic acid sequence:

TTCACCAATGCCTTCCG\hat{ }CTACGG, as denoted by SEQ ID NO.
103.
Before cut: ACCAATGCCTTCCGCTACGGC⁵⁰¹CACACCCTCATC,
as denoted by SEQ ID NO. 81.
After cut: ACCAATGCCTTCCGACTACGGC⁵⁰¹CACACCCTCATC,
as denoted by SEQ ID NO. 81.

(PAM sequence is underlined, \hat{ } marks cut site, Bold letters mark Codon that is substituted). A single stranded oligodeoxynucleotide (ssODN) is used as a template for HDR. ssODNs contain two homology arms flanking the new codon sequence. Homology arms are symmetric, with 50 bases from each side of the PAM.

Additionally, a PAM blocking mutation is introduced to the PAM sequence, in order to prevent CRISPR re-cutting.

The template ssODN oligo for G501S:

GACTCAGTGGACCCACGCATCGCCAACGTCTTCACCAATGCCTTCCGCTA
CAGC⁵⁰¹CACACCCTCATCCAACCCTTCATGTTCCGCCTGGACAATCGGT
ACCAGCC, as denoted by SEQ ID NO. 82.

Histidine502

Histidine 502 is the proximal ligand that coordinates the heme iron. Histidine 502 is critical for MPO heme binding.

A Histidine to Alanine substitution, H502A (Change CAC to GCC/GCA/GCT/GCG), was performed as shown by Example 4, using Cas9-mediated DSB within the codon sequence of Histidine 502, using the following gRNA sequence:

H502A-1: GGATGAGGGTGTGGCCG\hat{ }TAG, as denoted by SEQ ID NO. 35, that targets the protospacer that comprises the nucleic acid sequence

GGATGAGGGTGTGGCCG\hat{ } TAGCGG, as denoted by SEQ ID NO.
45.
Before cut: ACCAATGCCTTCCGCTACGGCCAC⁵⁰²ACCCTCATCC
A, as denoted by SEQ ID NO. 83.
After cut: ACCAATGCCTTCCGCTA\hat{ }CGGCCAC⁵⁰²ACCCTCATCC
A, as denoted by SEQ ID NO. 83.

(PAM sequence is underlined, \hat{ } marks cut site, Bold letters mark Codon that is substituted). A single stranded oligodeoxynucleotide (ssODN) was used as a template for HDR. ssODNs contain two homology arms flanking the new codon sequence. Homology arms are symmetric, with 50 bases from each side of the PAM.

Additionally, a PAM blocking mutation was introduced to the PAM sequence, in order to prevent CRISPR re-cutting.

The template ssODN oligo for H502A:

TACAATGACTCAGTGGACCCACGCATCGCCAACGTCTTCACCAATGCCTT
ACGCTACGGCGCC⁵⁰²ACCCTCATCCAACCCTTCATGTTCCGCCTGGACAA
TCGGT, as denoted by SEQ ID NO. 84.

Cysteine158

Cysteine 158 forms a disulfide bridge with Cysteine 319 in the pro-peptide, and its mutation disturbs MPO processing and release.

A Cysteine to Alanine substitution, C158A (Change TGC to GCC/GCA/GCT/GCG) is performed by Cas9-mediated DSB within the codon sequence of Cysteine 158, using the following gRNA sequence:

C158A-1: GTCAAGCGGCTGCGCCT\hat{ }ACC, as denoted by SEQ ID NO. 85. This gRNA targets the protospacer that comprises the nucleic acid sequence:

GTCAAGCGGCTGCGCCT\hat{ }ACCAGG as denoted by SEQ ID NO.
104.
Before cut: GTCCAAGTCAAGCGGCTGC¹⁵⁸GCCTACCAGGACGTGG
GG, as denoted by SEQ ID NO. 86.
After cut: GTCCAAGTCAAGCGGCTGC¹⁵⁸GCCT\hat{ }ACCAGGACGTGG
GG, as denoted by SEQ ID NO. 86.

(PAM sequence is underlined, \hat{ } marks cut site, Bold letters mark Codon that is substituted). A single stranded oligodeoxynucleotide (ssODN) is used as a template for HDR. ssODNs contain two homology arms flanking the new codon sequence. Homology arms are symmetric, with 50 bases from each side of the PAM.

Additionally, a PAM blocking mutation is introduced to the PAM sequence, in order to prevent CRISPR re-cutting.

The template ssODN oligo for C158A:

GACGCCCGCCCAGCTGAATGTGTTGTCCAAGTCAAGCGGCGCC¹⁵⁸GCCT
ACCATGACGTGGGGGTGACTTGCCCGGAGCAGGACAAATACCGCACCATC
ACCGGG, as denoted by SEQ ID NO. 87.

Asparagine355

Asparagine 355 is one of six Asparagine residues known to be glycosylated. Deglycosylation of Asparagine 355 was shown to lower MPO enzymatic activity.

An Asparagine to Alanine substitution, N355A (Change AAC to GCC/GCA/GCT/GCG), is performed using Cas9-mediated DSB within the codon sequence of Asparagine 355, using the following gRNA sequence:

N355A-1: GCTGGTTGGACATGTTG\hat{ }CGC, as denoted by SEQ ID NO. 88. This gRNA targets the protospacer that comprises the nucleic acid sequence:

GCTGGTTGGACATGTTG\hat{ }CGCAGG as denoted by SEQ ID NO.
105.
Before cut: CCCTGGCCAGGAACCTGCGCAAC³⁵⁵ATGTCCAACCAG
CTG, as denoted by SEQ ID NO. 89.
After cut: CCCTGGCCAGGAACCTGCG\hat{ }CAAC³⁵⁵ATGTCCAACCAG
CTG, as denoted by SEQ ID NO. 89.

(PAM sequence is underlined, \hat{ } marks cut site, Bold letters mark Codon that is substituted). A single stranded oligodeoxynucleotide (ssODN) is used as a template for HDR. ssODNs contain two homology arms flanking the new codon sequence. Homology arms are symmetric, with 50 bases from each side of the PAM.

Additionally, a PAM blocking mutation is introduced to the PAM sequence, in order to prevent CRISPR re-cutting.

The template ssODN oligo for N355A:

TTCGTGGACGCCAGCATGGTGTACGGCAGCGAGGAGCCCCTGGCCAGGAA
TCTGCGCGCC³⁵⁵ATGTCCAACCAGCTGGGGCTGCTGGCCGTCAACCAGC
GCTTCC, as denoted by SEQ ID NO. 90.

Arginine128

MPO propeptide cleavage by subtilisin-like propeptide convertases is necessary for MPO biosynthesis. Arginine 128 is part of subtilisin-like propeptide convertases-recognition site, and its substitution with a different amino acid should prevent propeptide cleavage.

An Arginine to Alanine substitution, R128A (Change AGG to GCC/GCA/GCT/GCG), is performed using Cas9-mediated DSB within the codon sequence of Arginine 128, using the following gRNA sequence:

R128A-1: TGGCTCTAGACCTGCTG\hat{ }GAGAGG, as denoted by SEQ ID NO. 91. This gRNA targets the protospacer that comprises the nucleic acid sequence:

TGGCTCTAGACCTGCTG\hat{ }GAGAGG, as denoted by SEQ ID NO.
106.
Before cut: TAGACCTGCTGGAGAGG¹²⁸AAGCTGCGGTCCCTG,
as denoted by SEQ ID NO. 92.
After cut: TAGACCTGCTG\hat{ }GAGAGG¹²⁸AAGCTGCGGTCCCTG,
as denoted by SEQ ID NO. 92.

(PAM sequence and coding sequence to be substituted is underlined, \hat{ } marks cut site).

A single stranded oligodeoxynucleotide (ssODN) is used as a template for HDR. ssODNs contain two homology arms flanking the new codon sequence. Homology arms are symmetric, with 50 bases from each side of the PAM.

Additionally, a PAM blocking mutation is introduced to the PAM sequence, in order to prevent CRISPR re-cutting.

The template ssODN oligo for R128A:

CGGCGGTGAGGGCCGCTGACTACCTGCACGTGGCTCTAGACCTGCTGGAG
GCG¹²⁸AAGCTGCGGTCCCTGTGGCGAAGGCCATTCAATGTCACTGATGT
GCTGAC, as denoted by SEQ ID NO. 93.

### Example 6

Hematopoietic Stem Cell Transplantation (HSCT) Combined with CRISPR-Mediated Gene Editing: Clinical Study

Assessment of the Patient's Eligibility

Patients are assessed for their physical eligibility for HSCT based on age, disease state, clinical evaluation and functional assessment such as the Karnofsky Performance Score. Patients are also sequenced at the MPO locus to validate their genetic compliance for MPO-targeted gRNA sequences.

CD34+ Mobilization and Collection by Apheresis

HSPCs can be extracted from iliac crest bone marrow aspirations or mobilized using a peripheral mobilization regimen combining chemotherapy with granulocyte-colony stimulating factor (G-CSF), CD34+ cells are collected and isolated from red and white blood cells by apheresis and stored in infusion bags. A critical number of 2×10⁶ cells per kilogram of the collected cells are cryopreserved with 20% DMSO as backup.

CD34+ Gene Editing and Enrichment of Transected Cells

Enriched CD34+ cells pre-stimulated with cytokines, are treated with gene editing vector targeting the MPO gene, such as electroporation with CRISPR-Cas9 Ribonucleoprotein complexed with the appropriate crRNA and fluorescently labeled tracrRNA. Following incubation with RNP complexes, CD34+ cell are enriched for tracrRNA-derived or vector-derived fluorescence or by other applicable selection method.

Quality Testing of Edited CD34+ Cells

MPO-edited CD34+ are inspected to ascertain bacterial or fungal contamination, endotoxin and mycoplasma as well as to assess cell viability. HSPC long-term repopulation capacity within CD34+ cells may be assessed by flow cytometry.

Gene-Edited Cell Expansion

MPO-edited CD34+ cells are ex-vivo expanded using compounds such as cytokines, metal chelators or small molecules may be necessary in order to reach critical cell number for transplantation or preferable for improving engraftment rate.

Conditioning Regimen

Before the infusion of gene-edited HSPCs, patients undergo a conditioning regimen including chemotherapy and ATG (anti-thymocyte globulin) with or without total body irradiation, to suppress bone marrow cells and prevent rejection of donor stem cells. This procedure may involve myeloablative or non-myeloablative regimens. In case of Autologous stem cell transplantation, after mobilization of CD34+ cells by G-CSF and apheresis of CD34+ cells, conditioning by Cytoxan and ATG with or without total body irradiation will be performed.

Cell Administration

Following HSPC assessment and conditioning regimen, patients are administered with a dose of at least 2×10⁶ cells per kilogram intravenously.

Post Transplantation Treatment and Monitoring

A post-implantation immunosuppression regimen, using drugs such as cyclosporine or tacrolimus, is given for the prevention of graft-versus-host disease (GVHD). Prophylactic medications are given for the prevention of severe infections. Patients are monitored with intense nursing care and treated for possible complications of HSCT or immunosuppression such as infections, GVHD etc. and potential side effects of the treatment. Hematopoietic recovery and engraftment rate are assessed and be hastened using growth factors.

### Example 7

MPO Knock-Out in the Treatment of Pulmonary Arterial Hypertension (PAH)

To evaluate the applicability of the suggested MPO KO undifferentiated bone marrow cells as a potential therapy on additional MPO-related conditions, the effect of MPO KO is next examined on a vascular disorder such as PAH in rat. Specifically, the model of Sugen5416/hypoxia-induced (SuHx-induced) pulmonary hypertension in rats is employed. SuHx cause a marked increase of vascular muscularization in small pulmonary arteries (PAs), increase of right ventricular systolic pressure (RVSP) and Right ventricular hypertrophy (RVH).

More specifically, SuHx rats are subjected to hematopoietic ablation by exposure to myeloablative irradiation, followed by re-population with bone marrow (BM) cells derived from either MPO-KO or WT rats. This results in four experimental groups: (1) WT with WT BM (WT-WT), (2) WT with MPO-KO BM (WT-MPO KO), (3) SuHx rats with WT BM (SuHx-WT) and (4) SuHx rats with MPO-KO BM (SuHx-MPO KO). In parallel experiments, the effect of MPO-KO BM is assessed in this rat PHA model using undifferentiated MPO-KO BM cells that are manipulated using the gene editing system of the invention.

To assess effect of the undifferentiated MPO-KO BM cells on PHA, activation and degranulation of polymorphonuclear neutrophils (PMN) is determined in the four experimental groups specified above. Still further, pulmonary artery pressure (PA pressure) and RVSP are determined, and mRNA of inflammation-related genes in lung tissue is also quantified in lung homogenates from rats of all experimental groups.

The effect of MPO KO on vasoconstrictive effect on the pulmonary vasculature is also tested by exposition of isolated pulmonary artery (PA) segments obtained from each experimental group to prostaglandin F₂α (PGF₂α). The role of MPO-dependent vasoconstriction in PAH is further examined by assessing the effective arterial elastance (Eₐ) from right ventricular pressure-volume loops. The effective Eₐ represents an established parameter of ventricular afterload, thus reflecting vascular resistance and compliance.

Immunohistochemical studies are conducted to compare the presence of increased vascular muscularization of pulmonary arterioles in all four experimental groups.

### Example 8

MPO Knock-Out in the Treatment of Crescentic Glomerulonephritis (CGN)

To evaluate the applicability of the suggested MPO KO undifferentiated bone marrow cells as a potential therapy on additional MPO-related conditions, the effect of MPO KO is next examined on crescentic glomerulonephritis (CGN), a syndrome signified by a precipitous loss of renal function, in a rat model. Specifically, rats preimmunized with human MPO-ANCA that are subsequently perfused with lysosomal extracts and H₂O₂ are known to develop CGN. Preimmunization with human MPO-ANCA causes IgG and C3 deposition and histologic glomerular injury may be observed which is proportional to the amount of IgG immune deposits. More specifically, preimmunized rats are subjected to hematopoietic ablation by exposure to myeloablative irradiation, followed by re-population with bone marrow (BM) cells derived from either MPO-KO or WT rats. This results in four experimental groups: (1) WT with WT BM (WT-WT), (2) WT with MPO-KO BM (WT-MPO KO), (3) Preimmunized rats with WT BM (Preimm-WT) and (4) Preimmunized rats with MPO-KO BM (Preimm-MPO KO). In parallel experiments, the effect of MPO-KO BM is assessed in this rat CGN model using undifferentiated MPO-KO BM cells that are manipulated using the gene editing system of the invention.

To assess effect of the undifferentiated MPO-KO BM cells on CGN, the kidneys of the rats are ablated and IgG and C3 deposition is examined as well as histologic glomerular injury, in the four experimental groups specified above.

1. A method of modulating the expression and/or activity of Myeloperoxidase (MPO) in a mammalian subject, the method comprises the step of administering to said subject an effective amount of at least one of:
(a) at least one gene editing compound adapted for modulating the expression and/or activity of MPO in at least one undifferentiated bone marrow (BM) cell of said subject; and
(b) at least one undifferentiated BM cell or undifferentiated BM cell population exhibiting a modulated expression and/or activity of MPO.

2. (canceled)

3. The method according to claim 1, wherein said at least one gene editing compound comprises at least one of at least one programmable engineered nuclease (PEN), any nucleic acid molecule comprising a sequence encoding said PEN, any kit, composition or vehicle comprising said at least one PEN, or the nucleic acid molecule encoding said PEN, optionally, wherein said PEN comprises at least one clustered regulatory interspaced short palindromic repeat (CRISPR)/CRISPR associated (cas) protein system, and wherein said method comprises the step of administering to said subject an effective amount of at least one of:
(a) at least one CRISPR/cas protein, or any nucleic acid molecule encoding said Cas protein; and
(b) at least one nucleic acid sequence comprising at least one guide RNA (gRNA) that targets a protospacer within the MPO gene, or any nucleic acid sequence encoding said gRNA; or any kit, composition or vehicle comprising at least one of (a) and (b).

4. (canceled)

5. The method according to claim 1, wherein said gRNA comprises the nucleic acid sequence as denoted by any one of SEQ ID NO: 33, SEQ ID NO: 34, SEQ ID NO: 35 SEQ ID NO: 42, SEQ ID NO: 47, SEQ ID NO: 50, SEQ ID NO: 55, SEQ ID NO: 60, SEQ ID NO: 63, SEQ ID NO: 66, SEQ ID NO: 71, SEQ ID NO: 74, SEQ ID NO: 77, SEQ ID NO: 80, SEQ ID NO: 85, SEQ ID NO: 88 and SEQ ID NO: 91.

6. The method according to claim 1, wherein said at least one undifferentiated BM cell or undifferentiated BM cell population is at least one undifferentiated BM cell or a BM cell population transduced or transfected with at least one gene editing compound capable of modulating the expression and/or activity of MPO in said cells optionally, the at least one undifferentiated BM cell or the undifferentiated BM cell population is of an autologous or of an allogenic source.

7. (canceled)

8. The method according to claim 1, wherein said undifferentiated BM cell population is a BM cell population of an allogeneic subject exhibiting an inhibited or eliminated expression and/or activity of MPO.

9. The method according to claim 1, wherein modulating the expression and/or activity of MPO comprises inhibiting or eliminating the expression and/or activity of MPO in said subject.

10. The method according to claim 1, for treating, preventing, ameliorating, inhibiting or delaying the onset of an MPO-related condition in a mammalian subject, said method comprises the step of administering to said subject a therapeutically effective amount of at least one of:
(a) at least one gene editing compound adapted for modulating the expression and/or activity of MPO in at least one undifferentiated BM cell of said subject; and
(b) at least one undifferentiated BM cell undifferentiated BM cell population exhibiting a modulated expression and/or activity of MPO.

11. The method according to claim 10, wherein said at least one undifferentiated BM cell or undifferentiated BM cell population is at least one undifferentiated BM cell or a BM cell population transduced or transfected with at least one gene editing compound capable of modulating the expression and/or activity of MPO in said cells, optionally, wherein the at least one undifferentiated BM cell or undifferentiated BM cell population is of an autologous or of an allogenic source.

12. (canceled)

13. The method according to claim 10, wherein said at least one undifferentiated BM cell or undifferentiated BM cell population is at least one undifferentiated BM cell or a BM cell population of an allogeneic subject exhibiting an inhibited or eliminated expression and/or activity of MPO.

14. The method according to claim 10, wherein said gene editing compound comprises at least one of at least one PEN, any nucleic acid molecule comprising a sequence encoding said PEN, any kit, composition or vehicle comprising said at least one PEN, or any nucleic acid sequence encoding said PEN optionally: wherein said at least one PEN comprises at least one CRISPR/Cas system, and wherein said method comprises the step of administering to said subject an effective amount of:
(a) at least one polypeptide comprising at least one Cas protein, or any nucleic acid sequence encoding said Cas protein; and
(b) at least one nucleic acid sequence comprising at least one gRNA that targets a protospacer within the MPO gene, or any nucleic acid sequence encoding said gRNA;
or any kit, composition or vehicle comprising at least one of (a) and (b).

15. (canceled)

16. The method according to claim 14, wherein the gRNA comprises the nucleic acid sequence as denoted by any one of SEQ ID NO: 33, SEQ ID NO: 34, SEQ ID NO: 35, SEQ ID NO: 42, SEQ ID NO: 47, SEQ ID NO: 50, SEQ ID NO: 55, SEQ ID NO: 60, SEQ ID NO: 63, SEQ ID NO: 66, SEQ ID NO: 71, SEQ ID NO: 74, SEQ ID NO: 77, SEQ ID NO: 80, SEQ ID NO: 85, SEQ ID NO: 88 and SEQ ID NO: 91.

17. The method according to claim 10, wherein said MPO-related condition is at least one of a neurodegenerative disorder, an immune-related disorder, a respiratory disorder, a proliferative disorder, a vascular disorder or any combination thereof.

18. The method according to claim 17, wherein at least one of:
(a) said neurodegenerative disorder is any one of Alzheimer's disease (AD) and Parkinson's disease (PD);
(b) said immune-related disorder is at least one of an autoimmune disorder and an inflammatory disorder;
(c) said autoimmune disorder is any one of multiple sclerosis (MS), Anti-neutrophil cytoplasmic antibodies (ANCAs)-related disorder, and systemic lupus erythematosus (SLE);
(d) said inflammatory disease is any one of atherosclerosis and Rheumatoid arthritis (RA);
(e) said respiratory disorder is Pulmonary arterial hypertension (PAH); and
(f) said proliferative disorder is cancer.

19-23. (canceled)

24. A pharmaceutical composition comprising a therapeutic effective amount of at least one of:
(a) at least one gene editing compound adapted for modulating the expression and/or activity of MPO in at least one undifferentiated BM cell of a subject in need thereof; and
(b) at least one undifferentiated BM cell or undifferentiated BM cell population exhibiting a modulated expression and/or activity of MPO; said composition optionally further comprises at least one of pharmaceutically acceptable carrier/s, diluent/s and/or excipient/s.

25. The pharmaceutical composition according to claim 24, wherein said gene editing compound is at least one PEN comprising at least one CRISPR/cas system, said CRISPR/cas system comprises at least one of:
(a) at least one CRISPR/cas protein, or any nucleic acid molecule encoding said Cas protein; and
(b) at least one nucleic acid sequence comprising at least one gRNA that targets a protospacer within the MPO gene, or any nucleic acid sequence encoding said gRNA; or any kit, composition or vehicle comprising at least one of (a) and (b) optionally, said gRNA comprises the nucleic acid sequence as denoted by any one of SEQ ID NO: 33, SEQ ID NO: 34, SEQ ID NO: 35 SEQ ID NO: 42, SEQ ID NO: 47, SEQ ID NO: 50, SEQ ID NO: 55, SEQ ID NO: 60, SEQ ID NO: 63, SEQ ID NO: 66, SEQ ID NO: 71, SEQ ID NO: 74, SEQ ID NO: 77, SEQ ID NO: 80, SEQ ID NO: 85, SEQ ID NO: 88 and SEQ ID NO: 91.

26. (canceled)

27. The pharmaceutical composition according to claim 24, wherein said at least one undifferentiated BM cell or undifferentiated BM cell population is:
(a) at least one undifferentiated BM cell a BM cell population transduced or transfected with at least one gene editing compound capable of modulating the expression and/or activity of MPO in said cells, optionally, said at least one undifferentiated BM cell or undifferentiated BM cell population is of an autologous or of an allogenic source; or
(b) at least one undifferentiated BM cell or undifferentiated BM cell population of an allogeneic subject exhibiting an inhibited or eliminated expression and/or activity of MPO.

28-29. (canceled)

30. The pharmaceutical composition according to claim 24, wherein said effective amount is adapted for use in a method for treating, preventing, ameliorating, inhibiting or delaying the onset of an MPO related condition or disease in a mammalian subject, optionally, the MPO-related condition is at least one of a neurodegenerative disorder, an immune-related disorder, a respiratory disorder a proliferative disorder, a vascular disorder or any combination thereof,
wherein at least one of:
(a) said neurodegenerative disorder is any one of AD and PD;
(b) said immune-related disorder is at least one of an autoimmune disorder and an inflammatory disorder;
(c) said autoimmune disorder is any one of multiple sclerosis (MS), Anti-neutrophil cytoplasmic antibodies (ANCAs)-related disorder, and systemic lupus erythematosus (SLE);
(d) said inflammatory disease is any one of atherosclerosis and Rheumatoid arthritis (RA);
(e) said respiratory disorder is Pulmonary arterial hypertension (PAH); and
(f) said proliferative disorder is cancer.

31-36. (canceled)

37. At least one undifferentiated BM cell transduced or transfected with at least one gene editing compound capable of modulating the expression and/or activity of MPO in said cell, optionally, said cell is of a subject suffering of an MPO-related condition.

38-43. (canceled)

44. The method according to claim 1 for inhibiting NETosis and related conditions in a mammalian subject, said method comprises the step of administering to said subject a therapeutically effective amount of at least one of:
(a) at least one gene editing compound adapted for modulating the expression and/or activity of MPO in at least one undifferentiated BM cell of said subject; and
(b) at least one undifferentiated BM cell or undifferentiated BM cell population exhibiting a modulated expression and/or activity of MPO, optionally, said gene editing compound is at least one PEN comprising at least one CRISPR/cas system, said CRISPR/cas system comprises at least one of:
(i) at least one CRISPR/cas protein, or any nucleic acid molecule encoding said Cas protein; and
(ii) at least one nucleic acid sequence comprising at least one gRNA that targets a protospacer within the MPO gene, or any nucleic acid sequence encoding said gRNA; or any kit, composition or vehicle comprising at least one of (i) and (ii).

45. (canceled)
